(19) United States
(12) Patent Application Publication
(10) Pub. No.: US 2013/0310427 A1
(43) Pub. Date: Nov. 21, 2013

(54) INHIBITORS OF HCV NS5A PROTEIN

(71) Applicants: Leping Li, San Francisco, CA (US); Min Zhong, San Francisco, CA (US)

(72) Inventors: Leping Li, San Francisco, CA (US); Min Zhong, San Francisco, CA (US)

(73) Assignee: PRESIDIO PHARMACEUTICALS, INC., San Francisco, CA (US)

(21) Appl. No.: 13/703,004

(22) PCT Filed: Jun. 9, 2011
(86) PCT No.: PCT/US11/39707

§ 371 Date: Jul. 18, 2013

Related U.S. Application Data
(60) Provisional application No. 61/353,166, filed on Jun. 9, 2010.

(51) Int. Cl. C07D 513/10; C07D 417/14; C07D 413/14; C07D 403/04

(52) U.S. Cl. CPC C07D 513/10; C07D 403/04; C07D 417/14; C07D 413/14
USPC 514/365; 548/306.1; 514/394; 548/313.1; 514/397; 548/200; 548/147; 548/236; 514/374

(57) ABSTRACT

Antiviral compounds may be used to inhibit or reduce the activity of Hepatitis C virus (HCV), particularly HCV's NS5A protein. In these contexts, inhibition and reduction of activity of the NS5A protein refers to a lower level of the measured activity relative to a control experiment in which the cells or the subjects are not treated with the test compound. The inhibition or reduction in the measured activity is at least a 10% reduction or inhibition. The compounds and their isomeric forms and pharmaceutically acceptable salts thereof are useful in treating and preventing HCV infection alone or when used in combination with other compounds targeting viral or cellular elements or functions involved in the HCV lifecycle.

This application claims priority to and the benefit of prior filed U.S. Provisional Application 61/353,166 filed on Jun. 9, 2010, which is hereby incorporated by reference for all purposes.

### FIELD OF THE INVENTION

The invention relates to compounds useful for inhibiting hepatitis C virus (“HCV”) replication, particularly functions of the non-structural 5A (“NS5A”) protein of HCV.

### BACKGROUND OF THE INVENTION

HCV is a single-stranded RNA virus that is a member of the Flaviviridae family. The virus shows extensive genetic heterogeneity as there are currently seven identified genotypes and more than 50 identified subtypes. In HCV infected cells, viral RNA is translated into a polyprotein that is cleaved into ten individual proteins. At the amino terminus are structural proteins: the core (C) protein and the envelope glycoproteins, E1 and E2. p7, an integral membrane protein, follows E1 and E2. Additionally, there are six non-structural proteins, NS2, NS3, NS4A, NS4B, NS5A and NS5B, which play a functional role in the HCV life cycle. (see, for example, Lindenbach, B.D. and C.M. Rice, Nature. 436:933-938, 2005).

Infection by HCV is a serious health issue. It is estimated that 170 million people worldwide are chronically infected with HCV. HCV infection can lead to chronic hepatitis, cirrhosis, liver failure and hepatocellular carcinoma. Chronic HCV infection is thus a major worldwide cause of liver-related premature mortality.

The present standard of care treatment regimen for HCV infection involves interferon-alpha, alone, or in combination with ribavirin. The treatment is cumbersome and sometimes has debilitating and severe side effects and many patients do not durably respond to treatment. New and effective methods of treating HCV infection are urgently needed.

### DETAILED DESCRIPTION OF THE INVENTION

Essential features of the NS5A protein of HCV make it an ideal target for inhibitors. The present disclosure describes a class of compounds targeting the NS5A protein and methods of their use to treat HCV infection in humans.

In a first aspect, compounds of formula I are provided:

D-A-B-A′-D′ wherein:

A and A′ are independently selected from the group consisting of

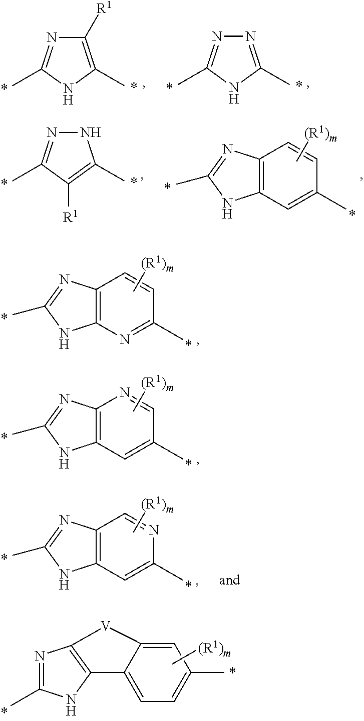

wherein
* indicates attachment points to the reminder of the compound,
R¹ is selected from the group consisting of C₁-C₄ alkyl, aryl, a halogen, —CN, —NO₂, —OR¹, —CF₃, —OCF₃, —OCHF₂, —CO₂R², —C(O)R³, —C(O)NR³R⁴, —NR³R⁴, —S(O)₂R², and —S(O)₂NR³R⁴,
m is 0, 1, or 3,
V is —CH₂—CH₂—, —CH═CH—, —N═CH—, (CH₂)ₐ—N(R³)—(CH₂)_{b}— or —(CH₂)ₐ—O—(CH₂)_{b}—, wherein a and b are independently 0, 1, 2, or 3 with the proviso that a and b are not both 0,
R², R³, and R⁴ are each independently chosen from the group consisting of hydrogen, C₁ to C₄ alkyl, C₁ to C₄ heteroalkyl, cycloalkyl, heterocycle, aryl, heteroaryl and aralkyl, and
wherein for each A and A′, B may be attached to either side of A and A′ so that in the example of A or A′ being

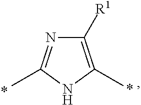

### the A-B-A′ can be any of:

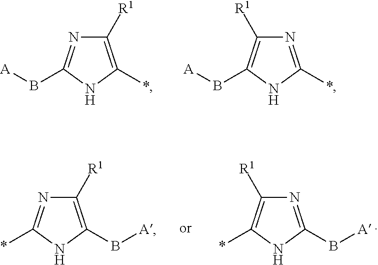 [0015] B is selected from the group consisting of a single bond, triple bond, [symbol] W, W [symbol] , [symbol] W [symbol] , W [symbol] W, W—W [symbol] , and W—W, wherein each W is independently selected from the group consisting of a cycloalkyl group, cycloalkenyl group, heterocyclic group, aryl group or heteroaryl group, with the proviso that when B is W—W, only one W is a six-membered aromatic ring;
D is

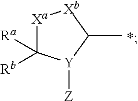

D′ is

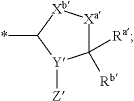

X^{a}, X^{b}, X^{a′}, and X^{b′} are each independently selected from the group consisting of C₂ to C₆ alkyl, C₂ to C₆ alkenyl, C₂ to C₆ heteroalkyl, and C₂ to C₆ heteroalkenyl, wherein:
   each hetero atom, if present, is independently N, O or S, and
   either or both of X^{a}—X^{b} and X^{a′}—X^{b′}, together with the atoms to which they are attached, optionally form a 4- to 9-membered ring which may be cycloalkyl and heterocycle and which may optionally be fused to another 3-5 membered ring;
R^{a}, R^{b}, R^{a′} and R^{b′} are each independently hydrogen, C₁ to C₈ alkyl or C₁ to C₈ heteroalkyl, wherein:
   each hetero atom, if present, is independently N, O or S,
   R^{a} and R^{b} are optionally joined, together with the atom to which they are attached, to form a 3- to 6-membered ring, and
   R^{a′} and R^{b′} are optionally joined, together with the atom to which they are attached, to form a 3- to 6-membered ring;
Y and Y′ are each independently N or CH; and
Z and Z′ are each independently selected from the group consisting of hydrogen, C₁ to C₈ alkyl, C₁ to C₈ heteroalkyl, cycloalkyl, heterocycle, aryl, heteroaryl, aralkyl, 1-3 amino acids,

—[U—(CR⁴₂)ₜ—NR⁵—(CR⁴₂)ₜ]ᵤ—U—(CR⁴₂)ₜ—NR⁷—(CR⁴₂)ₜ—R⁸,—U—(CR⁴₂)ₜ—R⁸ and

—[U—(CR⁴₂)ₜ—NR⁵—(CR⁴₂)ₜ]ᵤ—U—(CR⁴₂)ₜ—O—(CR⁴₂)ₜ—R⁸, wherein,
U is selected from the group consisting of —C(O)—, —C(S)— and —S(O)₂—,
each R⁴, R⁵ and R⁷ is independently selected from the group consisting of hydrogen, C₁ to C₈ alkyl, C₁ to C₈ heteroalkyl, cycloalkyl, heterocycle, aryl, heteroaryl and aralkyl,
R⁸ is selected from the group consisting of hydrogen, C₁ to C₈ alkyl, C₁ to C₈ heteroalkyl, cycloalkyl, heterocycle, aryl, heteroaryl, aralkyl, —C(O)—R⁸¹, —C(S)—R⁸¹, —C(O)—O—R⁸¹, —C(O)—N—R⁸¹₂, —S(O)₂—R⁸¹ and —S(O)₂—N—R⁸¹₂, wherein each R⁸¹ is independently chosen from the group consisting of hydrogen, C₁ to C₈ alkyl, C₁ to C₈ heteroalkyl, cycloalkyl, heterocycle, aryl, heteroaryl and aralkyl,
optionally, R⁷ and R⁸ together form a 4-7 membered ring,
each t is independently 0, 1, 2, 3, or 4, and
u is 0, 1, or 2.

In a first embodiment of the first aspect, A and A′ are selected from the group consisting of:

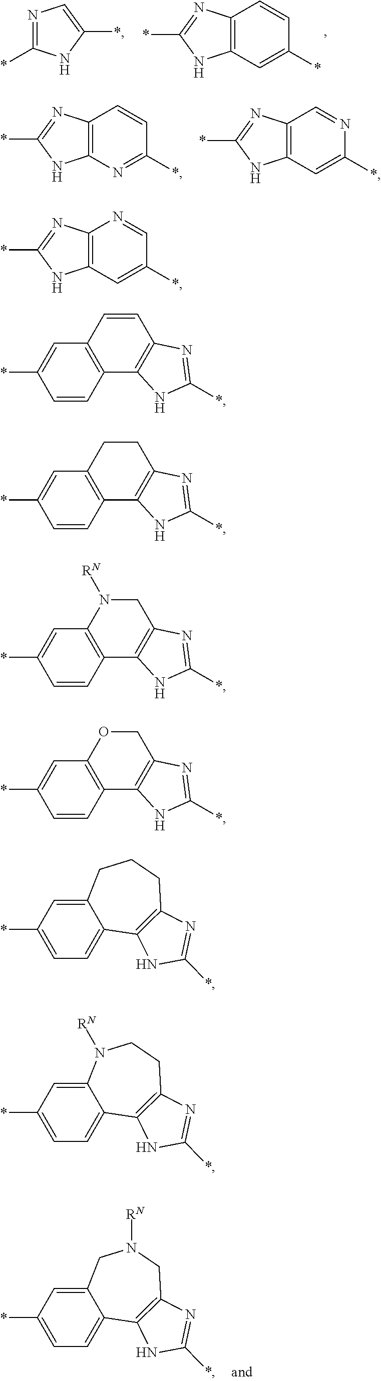

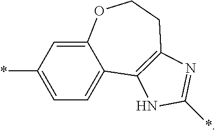

In a second embodiment of the first aspect, D is independently selected from group 1 and group 2. Group 1 consists of

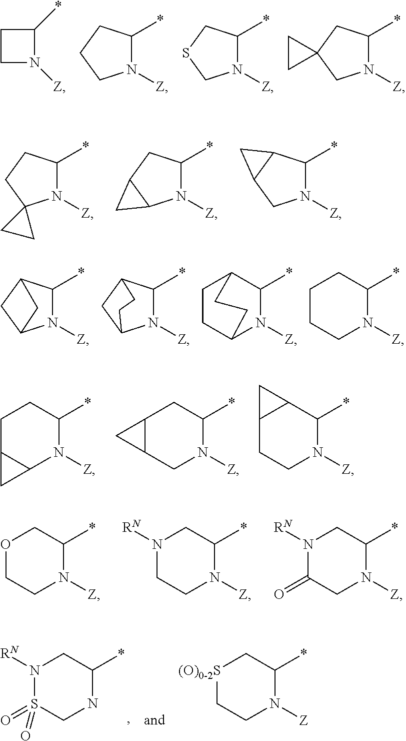

wherein R^{N} is independently selected from the group consisting of hydrogen, —OH, C₁ to C₁₂ alkyl, C₁ to C₁₂ heteroalkyl, cycloalkyl, heterocycle, aryl, heteroaryl, aralkyl, alkoxy, alkoxycarbonyl, alkanoyl, carbamoyl, substituted sulfonyl, sulfonate and sulfonamide. Group 2 consists of:

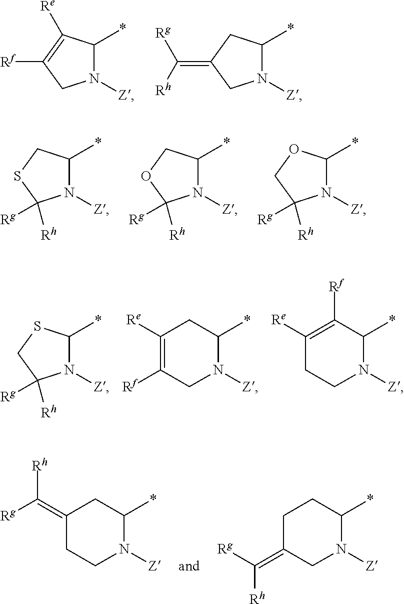

wherein R^{e}, R^{f}, R^{g}, and R^{h} are each independently hydrogen, C₁ to C₈ alkyl or C₁ to C₈ heteroalkyl, each hetero atom, if present, is independently N, O or S. R^{e} and R^{f} are optionally joined, together with the atom to which they are attached, to form a 5- to 8-membered ring, and R^{g} and R^{h} are optionally joined, together with the atom to which they are attached, to form a 3- to 8-membered ring.

In a third embodiment of the first aspect, D′ is independently selected from group 1′ and group 2′. Group 1′ consists of

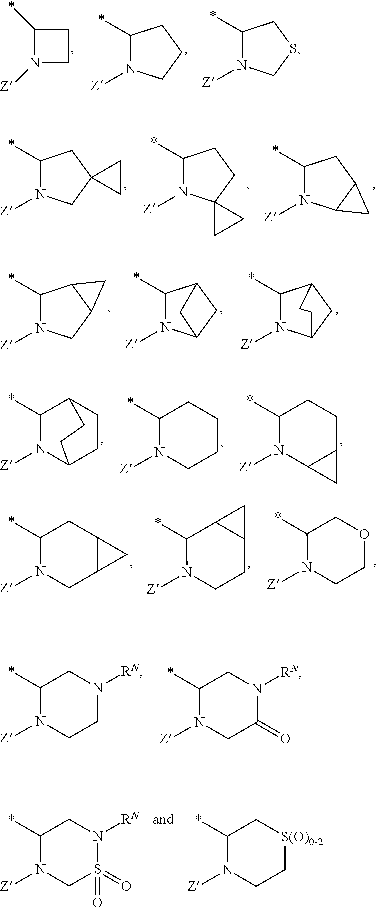

wherein R^{N} is independently selected from the group consisting of hydrogen, —OH, C₁ to C₁₂ alkyl, C₁ to C₁₂ heteroalkyl, cycloalkyl, heterocycle, aryl, heteroaryl, aralkyl, alkoxy, alkoxycarbonyl, alkanoyl, carbamoyl, substituted sulfonyl, sulfonate and sulfonamide. And Group 2′ consists of

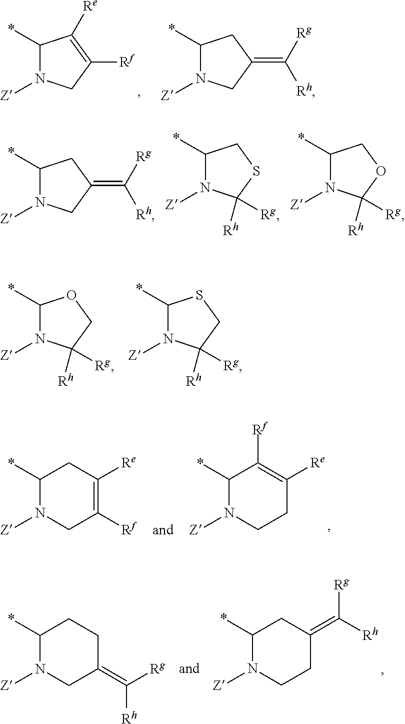

wherein R^{e}, R^{f}, R^{g}, and R^{h} are each independently hydrogen, C₁ to C₈ alkyl or C₁ to C₈ heteroalkyl, each hetero atom, if present, is independently N, O or S. R^{e} and R^{f} are optionally joined, together with the atom to which they are attached, to form a 5- to 8-membered ring, and R^{g} and R^{h} are optionally joined, together with the atom to which they are attached, to form a 3- to 8-membered ring.

In a fourth embodiment of the first aspect, if D is selected from Group 1, D′ is selected from Group 2′.

In a fifth embodiment of the first aspect, if D′ is selected from Group 1′, D is selected from Group 2.

In a sixth embodiment of the first aspect, A-B-A′ is selected from the group consisting of:

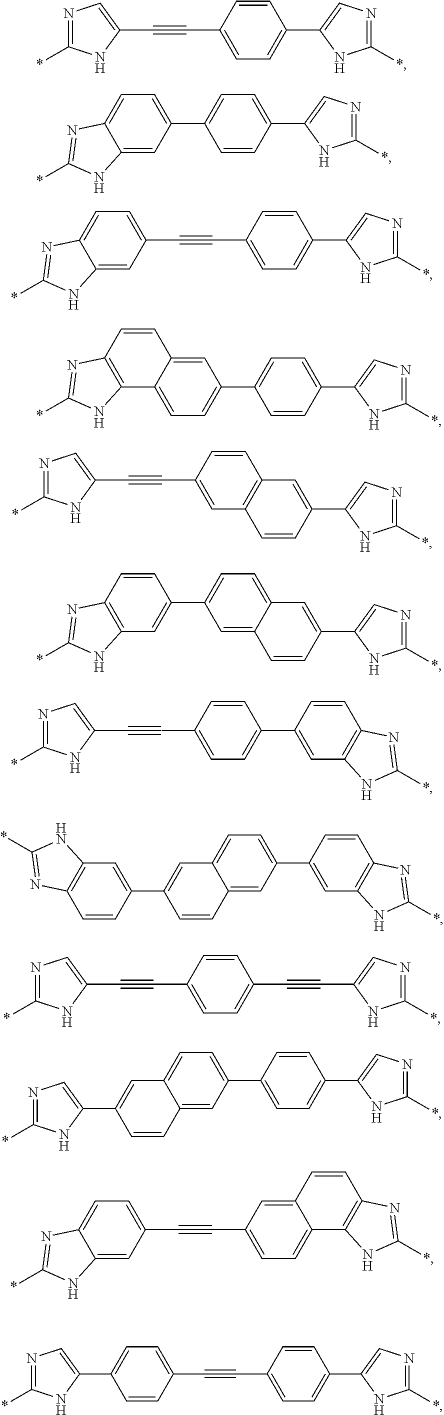

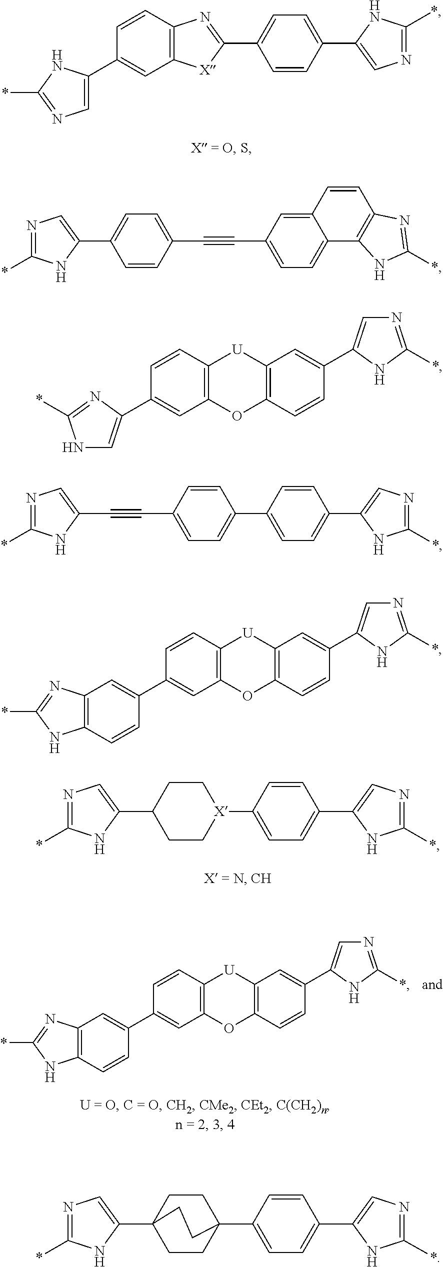

wherein * indicates attachment points to the reminder of the compound.

In a second aspect one or both of Y and Y′ in any of the previous aspects are N.

In a third aspect Z and Z′ in any of the previous aspects are each 1-3 amino acids.

In a first embodiment of the third aspect, the amino acids are all in the D or all in the L configuration.

In a second embodiment of the third aspect, Z and Z′ are each independently selected from the group consisting of

—[U—(CR⁴₂)ₜ—NR⁵—(CR⁴₂)ₜ]ᵤ—U—(CR⁴₂)ₜ—NR⁷—(CR⁴₂)ₜ—R⁸,

—U—(CR⁴₂)ₜ—R⁸ and —[U—(CR⁴₂)ₜ—NR⁵—(CR⁴₂)ₜ]ᵤ—U—(CR⁴₂)ₜ—O—(CR⁴₂)ₜ—R⁸.

In a third embodiment of the third aspect, one or both of Z and Z′ are —[U—(CR⁴₂)ₜ—NR⁵—(CR⁴₂)ₜ]ᵤ—U—(CR⁴₂)ₜ—NR⁷—(CR⁴₂)ₜ—R⁸.

In a fourth embodiment of the third aspect, one or both of Z and Z′ are —U—(CR⁴₂)ₜ—NR⁵—(CR⁴₂)ₜ—U—(CR⁴₂)ₜ—NR⁷—(CR⁴₂)ₜ—R⁸.

In a fifth embodiment of the third aspect, one or both of Z and Z′ are —U—(CR⁴₂)ₜ—NR⁷—(CR⁴₂)ₜ—R⁸.

In a sixth embodiment of the third aspect, one or both of Z and Z′ are —[C(O)—(CR⁴₂)ₜ—NR⁵—(CR⁴₂)ₜ]ᵤ—U—(CR⁴₂)ₜ—NR⁷—(CR⁴₂)ₜ—R⁸.

In a seventh embodiment of the third aspect, one or both of Z and Z′ are —C(O)—(CR⁴₂)ₜ—NR⁵—(CR⁴₂)ₜ—U—(CR⁴₂)ₜ—NR⁷—(CR⁴₂)ₜ—R⁸.

In an eighth embodiment of the third aspect, one or both of Z and Z′ are —[C(O)—(CR⁴₂)ₜ—NR⁵—(CR⁴₂)ₜ]ᵤ—C(O)—(CR⁴₂)ₜ—NR⁷—(CR⁴₂)ₜ—R⁸.

In a ninth embodiment of the third aspect, one or both of Z and Z′ are —C(O)—(CR⁴₂)ₜ—NR⁵—(CR⁴₂)ₜ—C(O)—(CR⁴₂)ₜ—NR⁷—(CR⁴₂)ₜ—R⁸.

In a tenth embodiment of the third aspect, one or both of Z and Z′ are —C(O)—(CR⁴₂)ₜ—NR⁷—(CR⁴₂)ₜ—R⁸.

In an eleventh embodiment of the third aspect, one or both of Z and Z′ are —C(O)—(CR⁴₂)ₙ—NR⁷—(CR⁴₂)C(O)—R⁸¹.

In a twelfth embodiment of the third aspect, one or both of Z and Z′ are —C(O)—(CR⁴₂)ₙ—NR⁷—C(O)—R⁸¹.

In a thirteenth embodiment of the third aspect, one or both of Z and Z′ are —C(O)—(CR⁴₂)ₙ—NR⁷—(CR⁴₂)C(O)—O—R⁸¹.

In a fourteenth embodiment of the third aspect, one or both of Z and Z′ are —C(O)—(CR⁴₂)ₙ—NR⁷—C(O)—O—R⁸¹.

In a fifteenth embodiment of the third aspect, one or both of Z and Z′ are —U—(CR⁴₂)ₜ—R⁸.

In a sixteenth embodiment of the third aspect, one or both of Z and Z′ are —C(O)—(CR⁴₂)ₜ—R⁸.

In a seventeenth embodiment of the third aspect, one or both of Z and Z′ are —[U—(CR⁴₂)ₜ—NR⁵—(CR⁴₂)ₜ]ᵤ—U—(CR⁴₂)ₜ—O—(CR⁴₂)ₜ—R⁸.

In an eighteenth embodiment of the third aspect, one or both of Z and Z′ are —U—(CR⁴₂)ₜ—NR⁵—(CR⁴₂)ₜ—U—(CR⁴₂)ₜ—O—(CR⁴₂)ₜ—R⁸.

In a nineteenth embodiment of the third aspect, one or both of Z and Z′ are —C(O)—(CR⁴₂)ₜ—NR⁵—(CR⁴₂)ₜ—C(O)—(CR⁴₂)ₜ—O—(CR⁴₂)ₜ—R⁸.

In a twentieth embodiment of the third aspect, one or both of Z and Z′ are —U—(CR⁴₂)ₜ—O—(CR⁴₂)ₜ—R⁸.

In a twenty-first embodiment of the third aspect, one or both of Z and Z′ are —C(O)—(CR⁴₂)ₜ—O—(CR⁴₂)ₜ—R⁸.

In a twenty-second embodiment of the third aspect, one or both of Z and Z′ are —C(O)—(CR⁴₂)ₙ—NR⁷R⁸ wherein R⁷ and R⁸ together form a 4-7 membered ring.

A fourth aspect of the invention provides a pharmaceutical composition comprising the compounds of the invention.

A fifth aspect of the invention provides use of the compounds of the invention in the manufacture of a medicament.

In a first embodiment of the fifth aspect the medicament is for the treatment of hepatitis C.

A sixth aspect of the invention provides a method of treating hepatitis C comprising administering to a subject in need thereof, a therapeutically effective amount of a compound of the invention.

### DETAILED DESCRIPTION

Unless otherwise stated, the following terms used in this application, including the specification and claims, have the definitions given below. It must be noted that, as used in the specification and the appended claims, the singular forms “a,” “an” and “the” include plural referents unless the context clearly dictates otherwise. Definition of standard chemistry terms may be found in reference works, including Carey and Sundberg (2007) “Advanced Organic Chemistry 5^{th} Ed.” Vols. A and B, Springer Science+Business Media LLC, New York. The practice of the present invention will employ, unless otherwise indicated, conventional methods of synthetic organic chemistry, mass spectroscopy, preparative and analytical methods of chromatography, protein chemistry, biochemistry, recombinant DNA techniques and pharmacology.

The term “alkanoyl” as used herein contemplates a carbonyl group with a lower alkyl group as a substituent.

The term “alkenyl” as used herein contemplates substituted or unsubstituted, straight and branched chain alkene radicals, including both the E- and Z-forms, containing from two to eight carbon atoms. The alkenyl group may be optionally substituted with one or more substituents selected from the group consisting of halogen, —CN, —NO₂, —CO₂R, —C(O)R, —O—R, —N(R^{N})₂, —N(R^{N})C(O)R, —N(R^{N})S(O)₂R, —SR, —C(O)N(R^{N})₂, —OC(O)R, —OC(O)N(R^{N})₂, —S(O)R, —SO₂R, —SO₃R, —S(O)₂N(R^{N})₂, phosphate, phosphonate, cycloalkyl, cycloalkenyl, aryl and heteroaryl.

The term “alkoxy” as used herein contemplates an oxygen with a lower alkyl group as a substituent and includes methoxy, ethoxy, butoxy, trifluoromethoxy and the like. It also includes divalent substituents linked to two separated oxygen atoms such as, without limitation, —O—(CH₂)₁₋₄—O—, —O—CF₂—O—, —O—(CH₂)₁₋₄—O—(CH₂CH₂—O)₁₋₄— and —(O—CH₂CH₂—O)₁₋₄—.

The term “alkoxycarbonyl” as used herein contemplates a carbonyl group with an alkoxy group as a substituent.

The term “alkyl” as used herein contemplates substituted or unsubstituted, straight and branched chain alkyl radicals containing from one to fifteen carbon atoms. The term “lower alkyl” as used herein contemplates both straight and branched chain alkyl radicals containing from one to six carbon atoms and includes methyl, ethyl, propyl, isopropyl, butyl, isobutyl, tert-butyl and the like. The alkyl group may be optionally substituted with one or more substituents selected from halogen, —CN, —NO₂, —C(O)₂R, —C(O)R, —O—R, —N(R^{N})₂, —N(R^{N})C(O)R, —N(R^{N})S(O)₂R, —SR, —C(O)N(R^{N})₂, —OC(O)R, —OC(O)N(R^{N})₂, —SOR, —SO₂R, —SO₃R, —S(O)₂N(R^{N})₂, phosphate, phosphonate, cycloalkyl, cycloalkenyl, aryl and heteroaryl.

The term “alkylene,” “alkenylene” and “alkynylene” as used herein refers to the groups “alkyl,” “alkenyl” and “alkynyl” respectively, when they are divalent, ie, attached to two atoms.

The term “alkylsulfonyl” as used herein contemplates a sulfonyl group which has a lower alkyl group as a substituent.

The term “alkynyl” as used herein contemplates substituted or unsubstituted, straight and branched carbon chain containing from two to eight carbon atoms and having at least one carbon-carbon triple bond. The term alkynyl includes, for example ethynyl, 1-propynyl, 2-propynyl, 1-butynyl, 3-methyl-1-butynyl and the like. The alkynyl group may be optionally substituted with one or more substituents selected from halo, —CN, —NO₂, —CO₂R, —C(O)R, —O—R, —N(R^{N})₂, —N(R^{N})C(O)R, —N(R^{N})S(O)₂R, —SR, —C(O)N(R^{N})₂, —OC(O)R, —OC(O)N(R^{N})₂, —SOR, —SO₂R, —SO₃R, —S(O)₂N(R^{N})₂, phosphate, phosphonate, cycloalkyl, cycloalkenyl, aryl and heteroaryl.

The term “amino” as used herein contemplates a group of the structure NR^{N}₂.

The term “amino acid” as used herein contemplates a group of the structure

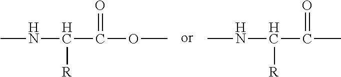

in either the D or the L configuration and includes but is not limited to the twenty “standard” amino acids: isoleucine, leucine, lysine, methionine, phenylalanine, threonine, tryptophan, valine, alanine, asparagine, aspartate, cysteine, glutamate, glutamine, glycine, proline, serine, tyrosine, arginine and histidine. The present invention also includes, without limitation, D-configuration amino acids, betamino acids, amino acids having side chains as well as all non-natural amino acids known to one skilled in the art.

The term “aralkyl” as used herein contemplates a lower alkyl group which has as a substituent an aromatic group, which aromatic group may be substituted or unsubstituted. The aralkyl group may be optionally substituted with one or more substituents selected from halogen, —CN, —NO₂, —CO₂R, —C(O)R, —O—R, —N(R^{N})₂, —N(R^{N})C(O)R, —N(R^{N})S(O)₂R, —SR, —C(O)N(R^{N})₂, —OC(O)R, —OC(O)N(R^{N})₂, —SOR, —SO₂R, —SO₃R, —S(O)₂N(R^{N})₂, phosphate, phosphonate, cycloalkyl, cycloalkenyl, aryl and heteroaryl.

The terms “aryl,” “aromatic group” or “aromatic ring” as used herein contemplates substituted or unsubstituted single-ring and multiple aromatic groups (for example, phenyl, pyridyl and pyrazole, etc.) and polycyclic ring systems (naphthyl and quinolinyl, etc.). The polycyclic rings may have two or more rings in which two atoms are common to two adjoining rings (the rings are “fused”) wherein at least one of the rings is aromatic, e.g., the other rings can be cycloalkyls, cycloalkenyls, aryl, heterocycles and/or heteroaryls. The aryl group may be optionally substituted with one or more substituents selected from halogen, alkyl, —CN, —NO₂, —CO₂R, —C(O)R, —O—R, —N(R^{N})₂, —N(R^{N})C(O)R, —N(R^{N})S(O)₂R, —SR, —C(O)N(R^{N})₂, —OC(O)R, —OC(O)N(R^{N})₂, —SOR, —SO₂R, —SO₃R, —S(O)₂N(R^{N})₂, —SiR₃, —P(O)R, phosphate, phosphonate, cycloalkyl, cycloalkenyl, aryl and heteroaryl.

The term “arylsulfonyl” as used herein contemplates a sulfonyl group which has as a substituent an aryl group. The term is meant to include, without limitation, monovalent as well as multiply valent aryls (eg, divalent aryls).

The term “carbamoyl” as used herein contemplates a group of the structure

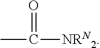

The term “carbonyl” as used herein contemplates a group of the structure

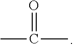

The term “carboxyl” as used herein contemplates a group of the structure

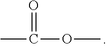

The term “cycloalkyl” as used herein contemplates substituted or unsubstituted cyclic alkyl radicals containing from three to twelve carbon atoms and includes cyclopropyl, cyclopentyl, cyclohexyl and the like. The term “cycloalkyl” also includes polycyclic systems having two rings in which two or more atoms are common to two adjoining rings (the rings are “fused”). The cycloalkyl group may be optionally substituted with one or more substituents selected from halo, —CN, —NO₂, —CO₂R, —C(O)R, —O—R, —N(R^{N})₂, —N(R^{N})C(O)R, —N(R^{N})S(O)₂R, —SR, —C(O)N(R^{N})₂, —OC(O)R, —OC(O)N(R^{N})₂, —SOR, —SO₂R, —S(O)₂N(R^{N})₂, phosphate, phosphonate, alkyl, cycloalkenyl, aryl and heteroaryl.

The term “cycloalkenyl” as used herein contemplates substituted or unsubstituted cyclic alkenyl radicals containing from four to twelve carbon atoms in which there is at least one double bond between two of the ring carbons and includes cyclopentenyl, cyclohexenyl and the like. The term “cycloalkenyl” also includes polycyclic systems having two rings in which two or more atoms are common to two adjoining rings (the rings are “fused”). The cycloalkenyl group may be optionally substituted with one or more substituents selected from halo, —CN, —NO₂, —CO₂R, —C(O)R, —O—R, —N(R^{N})₂, —N(R^{N})C(O)R, —N(R^{N})S(O)₂R, —SR, —C(O)N(R^{N})₂, —OC(O)R, —OC(O)N(R^{N})₂, —SOR, —SO₂R, —S(O)₂N(R^{N})₂, phosphate, phosphonate, alkyl, cycloalkenyl, aryl and heteroaryl.

The term “halo” or “halogen” as used herein includes fluorine, chlorine, bromine and iodine.

The term “heteroalkyl” as used herein contemplates an alkyl with one or more heteroatoms.

The term “heteroatom”, particularly within a ring system, refers to N, O and S.

The term “heterocyclic group,” “heterocycle” or “heterocyclic ring” as used herein contemplates substituted or unsubstituted aromatic and non-aromatic cyclic radicals having at least one heteroatom as a ring member. Preferred heterocyclic groups are those containing five or six ring atoms which includes at least one hetero atom and includes cyclic amines such as morpholino, piperidino, pyrrolidino and the like and cyclic ethers, such as tetrahydrofuran, tetrahydropyran and the like. Aromatic heterocyclic groups, also termed “heteroaryl” groups, contemplates single-ring hetero-aromatic groups that may include from one to three heteroatoms, for example, pyrrole, furan, thiophene, imidazole, oxazole, thiazole, triazole, pyrazole, oxodiazole, thiadiazole, pyridine, pyrazine, pyridazine, pyrimidine and the like. The term heteroaryl also includes polycyclic hetero-aromatic systems having two or more rings in which two or more atoms are common to two adjoining rings (the rings are “fused”) wherein at least one of the rings is a heteroaryl, e.g., the other rings can be cycloalkyls, cycloalkenyls, aryl, heterocycles and/or heteroaryls. Examples of polycyclic heteroaromatic systems include quinoline, isoquinoline, cinnoline, tetrahydroisoquinoline, quinoxaline, quinazoline, benzimidazole, benzofuran, benzothiophene, benzoxazole, benzothiazole, indazole, purine, benzotriazole, pyrrolepyridine, pyrrazolopyridine and the like. The heterocyclic group may be optionally substituted with one or more substituents selected from the group consisting of halo, alkyl, —CN, —NO₂, —CO₂R, —C(O)R, —O—R, —N(R^{N})₂, —N(R^{N})C(O)R, —N(R^{N})S(O)₂R, —SR, —C(O)N(R^{N})₂, —OC(O)R, —OC(O)N(R^{N})₂, —SOR, —SO₂R, —SO₃R, —S(O)₂N(R^{N})₂, —SiR₃, —P(O)R, phosphate, phosphonate, cycloalkyl, cycloalkenyl, aryl and heteroaryl.

The term “oxo” as used herein contemplates an oxygen atom attached with a double bond.

By “pharmaceutically acceptable” or “pharmacologically acceptable” is meant a material which is not biologically or otherwise undesirable, i.e., the material may be administered to an individual without causing any undesirable biological effects or interacting in a deleterious manner with any of the components of the composition in which it is contained.

“Pharmaceutically acceptable salt” refers to a salt of a compound of the invention which is made with counterions understood in the art to be generally acceptable for pharmaceutical uses and which possesses the desired pharmacological activity of the parent compound. Such salts include: (1) acid addition salts, formed with inorganic acids such as hydrochloric acid, hydrobromic acid, sulfuric acid, nitric acid, phosphoric acid and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl) benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid and the like; or (2) salts formed when an acidic proton present in the parent compound is replaced by a metal ion, e.g., an alkali metal ion, an alkaline earth ion or an aluminum ion; or coordinates with an organic base such as ethanolamine, diethanolamine, triethanolamine, N-methylglucamine, morpholine, piperidine, dimethylamine, diethylamine and the like. Also included are salts of amino acids such as arginates and the like, and salts of organic acids like glucurmic or galactunoric acids and the like (see, e.g., Berge et al., 1977, J. Pharm. Sci. 66:1-19).

The terms “phosphate” and “phosphonate” as used herein refer to the moieties having the following structures, respectively:

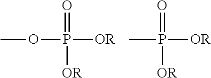

The terms “salts” and “hydrates” refers to the hydrated forms of the compound that would favorably affect the physical or pharmacokinetic properties of the compound, such as solubility, palatability, absorption, distribution, metabolism and excretion. Other factors, more practical in nature, which those skilled in the art may take into account in the selection include the cost of the raw materials, ease of crystallization, yield, stability, solubility, hygroscopicity, flowability and manufacturability of the resulting bulk drug.

The term sulfonamide as used herein contemplates a group having the structure

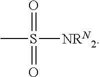

The term “sulfonate” as used herein contemplates a group having the structure

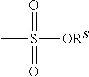

wherein R^{s} is selected from the group consisting of hydrogen, C₁-C₁₀ alkyl, C₂-C₁₀ alkenyl, C₂-C₁₀ alkynyl, C₁-C₁₀ alkanoyl or C₁-C₁₀ alkoxycarbonyl.

The term “sulfonyl” as used herein contemplates a group having the structure

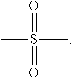

“Substituted sulfonyl” as used herein contemplates a group having the structure

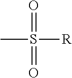

including, but not limited to alkylsulfonyl and arylsulfonyl.

The term “thiocarbonyl,” as used herein, means a carbonyl wherein an oxygen atom has been replaced with a sulfur.

Each R is independently selected from hydrogen, —OH, —CN, —NO₂, halogen, C₁ to C₁₂ alkyl, C₁ to C₁₂ heteroalkyl, alkenyl, alkynyl, cycloalkyl, heterocycle, aryl, heteroaryl, aralkyl, alkoxy, alkoxycarbonyl, alkanoyl, carbamoyl, substituted sulfonyl, sulfonate, sulfonamide, amino and oxo.

Each R^{N} is independently selected from the group consisting of hydrogen, —OH, C₁ to C₁₂ alkyl, C₁ to C₁₂ heteroalkyl, alkenyl, alkynyl, cycloalkyl, heterocycle, aryl, heteroaryl, aralkyl, alkoxy, alkoxycarbonyl, alkanoyl, carbamoyl, substituted sulfonyl, sulfonate and sulfonamide. Two R^{N} may be taken together with C, O, N or S to which they are attached to form a five to seven membered ring which may optionally contain a further heteroatom.

The compounds of the present invention may be used to inhibit or reduce the activity of HCV, particularly HCV's NS5A protein. In these contexts, inhibition and reduction of activity of the NS5A protein refers to a lower level of the measured activity relative to a control experiment in which the cells or the subjects are not treated with the test compound. In particular aspects, the inhibition or reduction in the measured activity is at least a 10% reduction or inhibition. One of skill in the art will appreciate that reduction or inhibition of the measured activity of at least 20%, 50%, 75%, 90% or 100% or any number in between, may be preferred for particular applications.

General Synthesis

The following abbreviations are used throughout this application:
ACN Acetonitrile
AcOH Acetic acid
aq Aqueous
Bn Benzyl
BnOH Benzyl alcohol
Boc t-Butoxycarbonyl
Cbz Benzoxylcarbonoyl
DCE Dichloroethane
DCM Dichloromethane
DEAD Diethyl azodicarboxylate
DEPBT 3-(Diethoxy-phosphoryloxy)-3H-benzo[d][1,2,3]triazin-4-one
DIEA (DIPEA) Diisopropylethylamine
DIBAL Diisobutylaluminium hydride
DMA N,N-Dimethylacetamide
DME 1,2-Dimethoxyethane
DMF N,N-Dimethylformamide
DMSO Dimethylsulfoxide
DMTMM 4-(4,6-Dimethoxy-1,3,5-triazin-2-yl)-4-methylmorpholinium chloride
DPPA Diphenylphosphoryl azide
dppp 1,3-Bis(diphenylphosphino)propane
dppf 1,1′-Bis(diphenylphosphino)ferrocene
DTT Dithiothreitol
EDCI 1-Ethyl-3-[3-(dimethylamino) propyl]carbodiimide hydrochloride
EDTA Ethylene diamine tetraacetic acid
EC₅₀ Effective concentration to produce 50% of the maximal effect
ESI Electrospray Ionization
Et₃N, TEA Triethylamine
EtOAc, EtAc Ethyl acetate
EtOH Ethanol
g Gram(s)
h or hr Hour(s)
HATU 2-(7-Aza-1H-benzotriazole-1-yl)-1,1,3,3-tetramethyluronium hexafluorophosphate
HBTU O-Benzotriazol-1-yl-N,N,N′,N′-tetramethyluronium hexafluorophosphate
Hex Hexanes
HOBt 1-Hydroxybenzotriazole
IC₅₀ The concentration of an inhibitor that causes a 50% reduction in a measured activity
LAH Lithium aluminum hydride
LDA Lithium diisopropylamide
LC-MS Liquid Chromatography Mass Spectrometry
mCPBA m-Chloroperoxybenzoic acid
MeI Methyl Iodide
MeOH Methanol
min Minute(s)
mmol Millimole(s)
Moc Methoxylcarbonyl
NMM 4-Methylmorpholine
NMP N-methylpyrrolidinone
PG Protective Group
PTT Phenyl trimethyl tribromide
Py, Pyr Pyridine
rt Room temperature
TEA Triethylamine
Tf Trifluoromethanesulfonate
TFA Trifluoroacetic acid
TFAA Trifluoroacetic anhydride
THF Tetrahydrofuran
TLC Thin Layer Chromatography
TMSOTf Trimethylsilyl trifluoromethanesulfonate

Reagents and solvents used below can be obtained from commercial sources such as Aldrich Chemical Co. (Milwaukee, Wis., USA). ¹HNMR spectra were recorded on a Bruker 400 MHz or 500 MHz NMR spectrometer. Significant peaks are tabulated in the order: chemical shift, multiplicity (s, singlet; d, doublet; t, triplet; q, quartet; m, multiplet; br s, broad singlet), coupling constant(s) in Hertz (Hz) and number of protons.

The following examples are provided by way of illustration only and not by way of limitation. Those skilled in the art will readily recognize a variety of noncritical parameters that could be changed or modified to yield essentially similar results. Efforts have been made to ensure accuracy with respect to numbers used (e.g., amounts, temperatures, etc.), but some experimental errors and deviations should, of course, be allowed for.

Liquid chromatography mass spectra (LC-MS) were typically obtained using an electrospray ionization (ESI) source in either the positive or negative mode.

The compounds were named using ChemDraw program from CambridgeSoft Inc.

The compounds and processes of the present invention will be better understood through the following examples. The schemes and procedures exemplify some of the synthetic routes that can be used for the preparation of compounds and their analogs in this invention. The examples are provided by way of illustration only and not by way of limitation. Those skilled in the art will readily recognize a variety of noncritical parameters that could be changed or modified to yield essentially similar results. Alternative reagents for a given transformation are also possible. Efforts have been made to ensure accuracy with respect to numbers used (e.g., amounts, temperatures, etc.), but some experimental errors and deviations should, of course, be allowed for.

### Preparation of Key Building Blocks

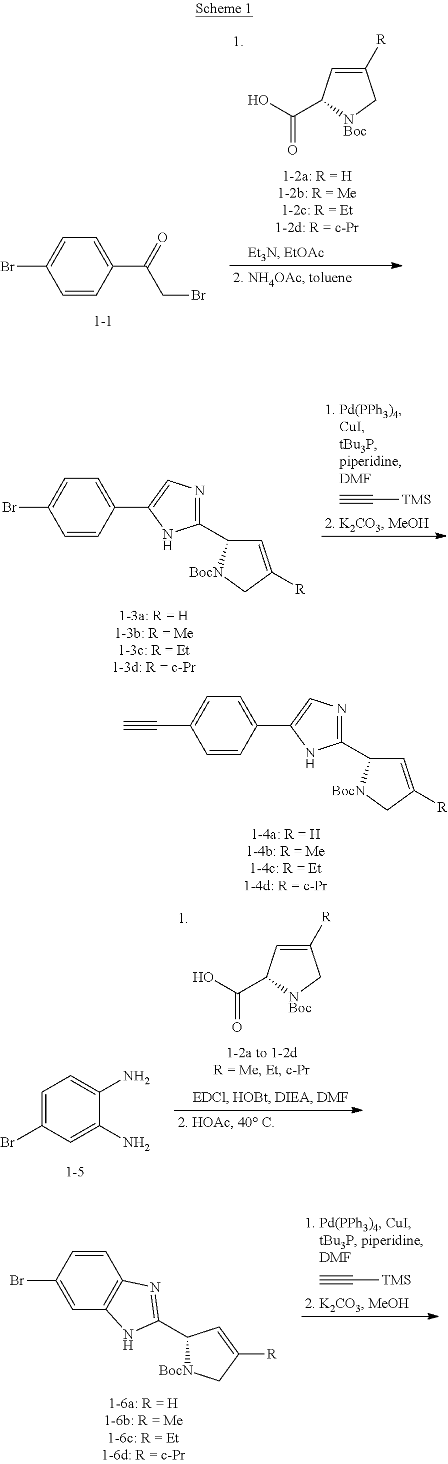

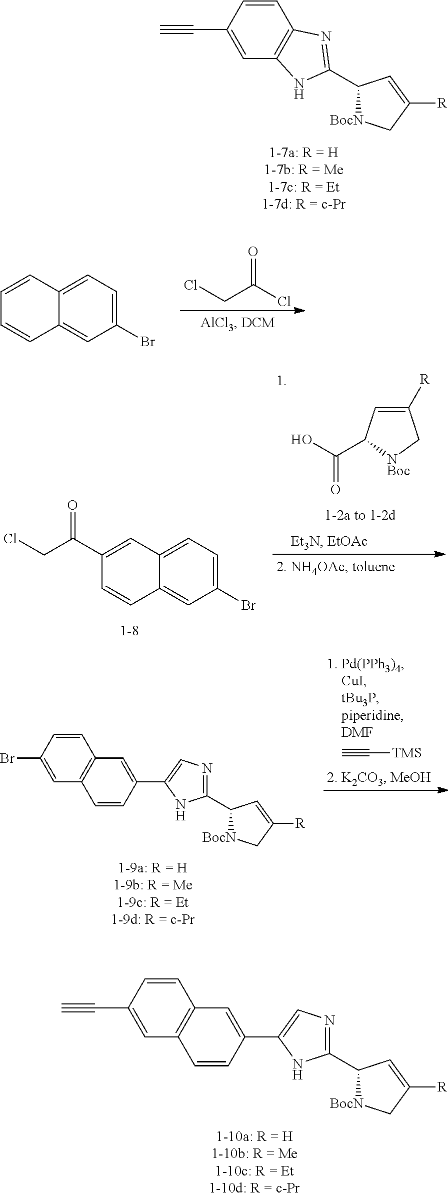

### (S)-1-(tert-butoxycarbonyl)-4-methyl-2,5-dihydro-1H-pyrrole-2-carboxylic acid (1-2b)

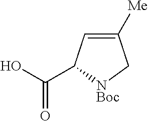 [SMILES: CC1=C[C@@H](C(=O)O)N(C(=O)OC(C)(C)C)C1]

The following procedures (Step 1 to 3) were utilized to prepare N-protected 2,5-dihydro-1H-pyrrole-2-carboxylic acids bearing various types of 4-substituents, including those represented by compounds 1-2a, 1-2b, 1-2c, and 1-2d. Other dihydropyrrole compounds bearing different substituents and substitution patterns may also be prepared similarly.

Step 1.

To a stirred solution of sodium bis(trimethylsilyl)amide (1 N in THF, 45.2 mL, 45.2 mmol) was added dropwise a solution of (S)-1-tert-butyl 2-methyl 4-oxopyrrolidine-1,2-dicarboxylate (10 g, 41.1 mmol, prepared as described in Tetrahedron, 51(14), 4195-212; 1995) in THF (50 mL) at −78° C. After 20 mins, N-phenyl-bis(trifluoromethanesulfonimide) (15.4 g, 43.2 mmol) was added, and the reaction mixture was stirred at −78° C. for another 3 hrs. After being quenched with aqueous NaHCO₃ the reaction mixture was extracted with EtOAc. The organic layer was washed with brine, dried over anhydrous Na₂SO₄, and concentrated in vacuo. The residue was purified by flash column chromatography (Hex/EtOAc=9/1 (v/v)) to afford (S)-1-tert-butyl 2-methyl 4-(trifluoromethylsulfonyloxy)-1H-pyrrole-1,2(2H,5H)-dicarboxylate (14.8 g, 96% yield) as a yellow oil. ¹H NMR (300 MHz, CDCl₃): δ 5.72 (dd, 1H), 5.02 (m, 1H), 4.28-4.42 (m, 2H), 3.77 (s, 3H), 1.42-1.47 (m, 9H) ppm.

Step 2.

To a solution of (S)-1-(tert-butoxycarbonyl)-4-(trifluoromethylsulfonyloxy)-2,5-dihydro-1H-pyrrole-2-carboxylic acid (5.00 g, 13.3 mmol) in dioxane (75 mL) was added methylboronic acid (1.0 g, 16.6 mmol), Pd[PPh₃]₄ (0.465 g, 0.402 mmol) and Na₂CO₃ (2 M in H₂O, 15 mL). After being thoroughly degassed the reaction mixture was heated at 95° C. for 2.5 hrs under a N₂ atmosphere. The reaction mixture was cooled to rt and concentrated in vacuo. The residue was diluted in EtOAc and washed with H₂O and brine, respectively. The organic layer was dried over anhydrous Na₂SO₄, filtered and concentrated in vacuo. The remaining residue was purified by flash column chromatography (Hex/EtOAc=5/1 (v/v)) to afford (S)-1-tert-butyl 2-methyl 4-methyl-1H-pyrrole-1,2(2H,5H)-dicarboxylate (2.25 g, 70% yield) as a colorless oil. ¹H NMR (300 MHz, CDCl₃): δ 5.36 (dd, 1H), 4.90 (m, 1H), 4.04-4.16 (m, 2H), 3.72 (m, 3H), 1.79 (m, 3H), 1.42-1.47 (m, 9H) ppm.

Step 3.

To a solution of (S)-1-tert-butyl 2-methyl 4-methyl-1H-pyrrole-1,2(2H,5H)-dicarboxylate (3.76 g, 15.6 mmol) in THF (20 mL), MeOH (15 mL) and H₂O (15 mL) was added LiOH.H₂O (1.30 g, 31.2 mmol). The reaction was stirred at rt overnight. The mixture was concentrated in vacuo and water (15 mL) was added. The solution was washed with Et₂O, acidified with 6 N HCl to pH 3 and extracted with DCM (2×100 mL). The combined DCM extracts were dried over anhydrous Na₂SO₄, filtered and concentrated to give (S)-1-(tert-butoxycarbonyl)-4-methyl-2,5-dihydro-1H-pyrrole-2-carboxylic acid (1-2b) (3.5 g, quantitative yield) as a colorless oil. LC-MS (ESI): m/z 226 [M−H]⁻.

### (S)-1-(tert-butoxycarbonyl)-4-cyclopropyl-2,5-dihydro-1H-pyrrole-2-carboxylic acid (1-2d)

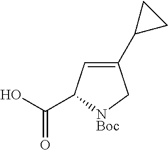 [SMILES: CC(C)(C)OC(=O)N1CC(C2CC2)=C[C@H]1C(=O)O]

Compound 1-2d was prepared by using the conditions described above and substituting cyclopropylboronic acid for methylboronic acid in Step 2.

Step a. To a solution of (S)-1-tert-butyl 2-methyl 4-(trifluoromethylsulfonyloxy)-1H-pyrrole-1,2(2H,5H)-dicarboxylate (15 g, 40 mmol) in dioxane (250 mL) was added cyclopropylboronic acid (5.15 g, 60 mmol), Pd(PPh₃)₄ (2.31 g, 2.0 mmol) and Na₂CO₃ (2 N in H₂O, 45 mL). The flask was degassed and heated at 100° C. for 3 hr under N₂ atmosphere. The reaction mixture was cooled to rt and concentrated in vacuo. The residue was diluted in EtOAc and washed with H₂O, brine. The organic layer was dried with anhydrous Na₂SO₄ and concentrated. The resulting residue was purified by flash column chromatography (Hex/EtOAc=5/1 (v/v)) to afford (S)-1-tert-butyl 2-methyl 4-methyl-1H-pyrrole-1,2(2H,5H)-dicarboxylate (4.0 g) as a colorless oil. ¹H NMR (300 MHz, CDCl₃): δ 5.30 (m, 1H), 4.90 (m, 1H), 4.13-3.95 (m, 2H), 3.72-3.70 (m, 3H), 1.47-1.42 (m, 9H), 1.32-1.25 (m, 1H), 0.77-0.73 (m, 2H), 0.55-0.53 (m, 2H) ppm.

Step b.

To a solution of (S)-1-tert-butyl 2-methyl 4-methyl-1H-pyrrole-1,2(2H,5H)-dicarboxylate from above (3.70 g, 13.8 mmol) in THF (20 mL), MeOH (15 mL) and H₂O (15 mL) was added LiOH.H₂O (1.30 g, 30.9 mmol). The reaction was stirred at rt overnight. The mixture was concentrated in vacuo and water (15 mL) was added. The solution was washed with Et₂O, acidified with 6 N HCl to pH 3. The aqueous phase was extracted with DCM. The combined organic phase was dried with anhydrous Na₂SO₄ and concentrated to give 25 (3.5 g, quantitative yield) as a colorless oil. LC-MS (ESI): m/z 252 [M−H]⁻.

### (S)-tert-butyl 2-(5-(4-bromophenyl)-1H-imidazol-2-yl)-4-methyl-2,5-dihydro-1H-pyrrole-1-carboxylate (1-3b)

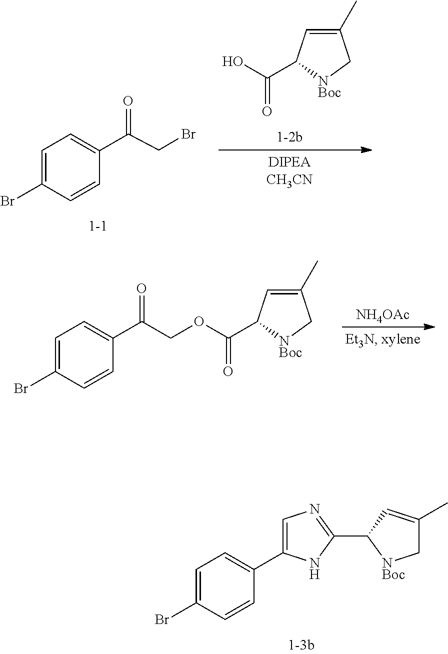 [SMILES: O=C(CBr)c1ccc(Br)cc1 ; CC1=C[C@@H](C(=O)O)N(C(=O)OC(C)(C)C)C1 ; CC1=C[C@@H](C(=O)OCC(=O)c2ccc(Br)cc2)N(C(=O)OC(C)(C)C)C1 ; CC1=C[C@@H](c2ncc(-c3ccc(Br)cc3)[nH]2)N(C(=O)OC(C)(C)C)C1 ; "NH4OAc"]

General Procedure A:

Steps 1 and 2, synthesis of a 2,5-disubstituted imidazole from an α-bromoketone (or α-chloroketone) and a carboxylic acid.

Step 1.

A solution of 2-bromo-1-(4-bromophenyl)ethanone (1-1) (2.27 g, 10.0 mmol) in CH₃CN (30 mL) was added (S)-1-(tert-butoxycarbonyl)-4-methyl-2,5-dihydro-1H-pyrrole-2-carboxylic acid (1-2b) (3.05 g, 11.0 mmol) and DIPEA (3.30 mL, 20 mmol). The resulting mixture was stirred at rt overnight. The volatile components were removed in vacuo, and the residue was partitioned between water and DCM. The organic layer was dried over anhydrous Na₂SO₄, filtered, and concentrated. The crude product was purified by flash column chromatography (Hex/EtOAc=4/1 (v/v)) to afford (S)-2-(2-(4-bromophenyl)-2-oxoethyl) 1-tert-butyl 4-methyl-1H-pyrrole-1,2(2H,5H)-dicarboxylate (3.65 g, 86% yield). LC-MS (ESI): m/z 426 [M+H]⁺.

Step 2.

To a solution of (S)-2-(2-(4-bromophenyl)-2-oxoethyl) 1-tert-butyl 4-methyl-1H-pyrrole-1,2(2H,5H)-dicarboxylate (3.65 g, 8.6 mmol) in xylene (90 mL) in a sealed tube was added ammonium acetate (10.4 g, 135 mmol) and triethylamine (18.8 mL, 135 mmol). The resulting mixture was stirred at 140° C. for 2 hrs. Analysis by LC-MS showed the reaction was completed. The solvent was removed in vacuo, and the residue was partitioned between water and DCM. The aqueous layer was back extracted with DCM. The combined organic phase was dried with anhydrous Na₂SO₄, filtered, and concentrated. The crude mixture was purified by flash column chromatography (Hex/EtOAc=1/1 (v/v)) to afford the compound 1-3b (2.5 g, 72% yield). LC-MS (ESI): m/z 406 [M+H]⁺.

### Synthesis of (S)-tert-butyl 2-(5-(4-ethynylphenyl)-1H-imidazol-2-yl)-4-methyl-2,5-dihydro-1H-pyrrole-1-carboxylate (1-4b)

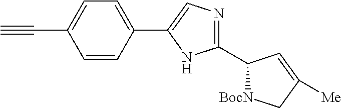 [SMILES: C#Cc1ccc(-c2cnc([C@@H]3C=C(C)CN3C(=O)OC(C)(C)C)[nH]2)cc1]

General Procedure B:

Steps 1 and 2, synthesis of an arylacetylyne by Sonogoshira Reaction.

Step 1.

To a solution of 1-3b (10.0 g, 24.8 mmol) in anhydrous THF (100 mL) was added PPh₃ (1.34 g, 5.11 mmol), Pd[PPh₃]₂Cl₂ (1.79 g, 2.56 mmol), CuI (0.24 g, 1.28 mmol), DIPEA (7.75 g, 76.8 mmol), and TMS-acetylene (5.02 g, 51.2 mmol). The mixture was refluxed under argon overnight. At the completion of the reaction, volatile solvents were removed under reduced pressure; the residue was treated with water, extracted with EtOAc (2×100 mL). The combined organic phases were dried, filtered, and concentrated. The residue was purified by silica gel column chromatography (petroleum ether/EtOAc=3/1 (v/v)) to afford (S)-tert-butyl 4-methyl-2-(5-(4-((trimethylsilyl)ethynyl)phenyl)-1H-imidazol-2-yl)-2,5-dihydro-1H-pyrrole-1-carboxylate (5.80 g, 55% yield) as a yellow solid. LC-MS (ESI): m/z 427 [M+H]⁺.

Step 2.

A solution of (S)-tert-butyl 4-methyl-2-(5-(4-((trimethylsilyl)ethynyl)phenyl)-1H-imidazol-2-yl)-2,5-dihydro-1H-pyrrole-1-carboxylate (5.80 g, 13.6 mmol) in THF (100 mL) and MeOH (100 mL) was treated with K₂CO₃ (5.85 g, 42.4 mmol) at rt for 3 hrs. The mixture was filtered, the filtrate was concentrated under reduced pressure and the residue was purified by silica gel column chromatography (DCM/MeOH=40/1 (v/v)) to afford (S)-tert-butyl 2-(5-(4-ethynylphenyl)-1H-imidazol-2-yl)-4-methyl-2,5-dihydro-1H-pyrrole-1-carboxylate (1-4b) (3.80 g, 80% yield) as a yellow solid. LC-MS (ESI): m/z 450 [M+H]⁺.

### (S)-tert-butyl 2-(5-(4-bromophenyl)-1H-imidazol-2-yl)-4-cyclopropyl-2,5-dihydro-1H-pyrrole-1-carboxylate (1-3d)

LC-MS (ESI): m/z 430 [M+H]⁺.

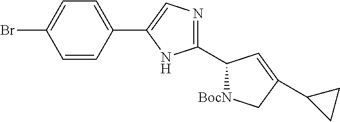 [SMILES: CC(C)(C)OC(=O)N1CC(C2CC2)=C[C@H]1c1ncc(-c2ccc(Br)cc2)[nH]1]

### (S)-tert-butyl 4-cyclopropyl-2-(5-(4-ethynylphenyl)-1H-imidazol-2-yl)-2,5-dihydro-1H-pyrrole-1-carboxylate (1-4d)

LC-MS (ESI): m/z 376 [M+H]⁺.

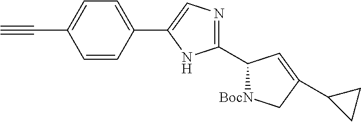 [SMILES: C#Cc1ccc(-c2cnc([C@@H]3C=C(C4CC4)CN3C(=O)OC(C)(C)C)[nH]2)cc1]

### (S)-tert-butyl 2-(6-bromo-1H-benzo[d]imidazol-2-yl)-4-methyl-2,5-dihydro-1H-pyrrole-1-carboxylate (1-6b)

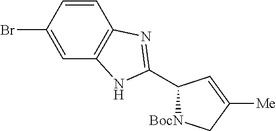 [SMILES: CC1=C[C@@H](c2nc3ccc(Br)cc3[nH]2)N(C(=O)OC(C)(C)C)C1]

General Procedure C:

synthesis of benzoimidazole from 1,2-benzenediamine. To a solution of (5)-1-(tert-butoxycarbonyl)-4-methyl-2,5-dihydro-1H-pyrrole-2-carboxylic acid (1-2b) (0.95 g, 4.19 mmol) in THF (15 mL) was added DIPEA (2.0 mL, 12.1 mmol). The mixture was stirred at room temperature for 10 min. EDAC (0.80 g, 4.19 mmol) was added to the solution. The resulting solution was stirred for another 1 hr, 4-bromobenzene-1,2-diamine (1-5) (0.87 g, 4.65 mmol) was added. The mixture was stirred at 30° C. overnight. The solvent was removed in vacuo, and the residue was partitioned between water and EtOAc. The aqueous layer was extracted with EtOAc. The combined organic phases were dried (Na₂SO₄), filtered, and concentrated. The crude acylated product was dissolved in AcOH (15 mL), and the mixture was stirred at 40° C. overnight. The solvent was concentrated in vacuo. The residue was re-dissolved in EtOAc and washed with NaHCO₃, H₂O and brine. The organic phase was dried with anhydrous Na₂SO₄, filtered, and concentrated. The crude mixture was purified by flash column chromatography (EtOAc/DCM=1/20 to 1/10 (v/v)) to afford compound 1-6b (420 mg). LC-MS (ESI): m/z 378 [M+H]⁺.

### (S)-tert-butyl 2-(6-ethynyl-1H-benzo[d]imidazol-2-yl)-4-methyl-2,5-dihydro-1H-pyrrole-1-carboxylate (1-7b)

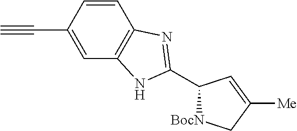 [SMILES: C#Cc1ccc2nc([C@@H]3C=C(C)CN3C(=O)OC(C)(C)C)[nH]c2c1]

Prepared by following General Procedure B. LC-MS (ESI): m/z 324 [M+H]⁺.

### (S)-tert-butyl 2-(6-bromo-1H-benzo[d]imidazol-2-yl)-4-cyclopropyl-2,5-dihydro-1H-pyrrole-1-carboxylate (1-6d)

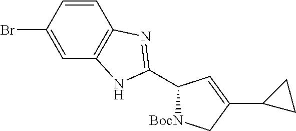 [SMILES: CC(C)(C)OC(=O)N1CC(C2CC2)=C[C@H]1c1nc2ccc(Br)cc2[nH]1]

Prepared by following General Procedure C. LC-MS (ESI): m/z 404 [M+H]⁺

### (S)-tert-butyl 2-(6-ethynyl-1H-benzo[d]imidazol-2-yl)-4-cyclopropyl-2,5-dihydro-1H-pyrrole-1-carboxylate (1-7d)

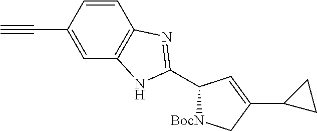 [SMILES: C#Cc1ccc2nc([C@@H]3C=C(C4CC4)CN3C(=O)OC(C)(C)C)[nH]c2c1]

Prepared by following General Procedure B. LC-MS (ESI): m/z 350 [M+H]⁺

### (S)-tert-butyl 2-(5-(6-bromonaphthalen-2-yl)-1H-imidazol-2-yl)-4-methyl-2,5-dihydro-1H-pyrrole-1-carboxylate (1-9b)

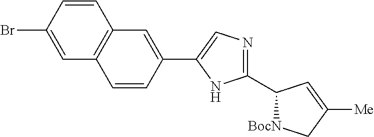 [SMILES: CC1=C[C@@H](c2ncc(-c3ccc4cc(Br)ccc4c3)[nH]2)N(C(=O)OC(C)(C)C)C1]

Step 1.

Again referring to route outlined in Scheme 1, (General Procedure A) a solution of 1-(6-bromonaphthalen-2-yl)-2-chloroethanone (1-8) (1.18 g, 4.15 mmol, prepared from 2-bromo-naphthalene via a Friedel-Craft reaction with chloroacetyl chloride) in CH₃CN (40 mL) was added (S)-4-methyl-2,5-dihydro-pyrrole-1,2-dicarboxylic acid 1-tert-butyl ester (940 mg, 4.15 mmol) and N,N-diisopropylethylamine (0.73 mL, 4.15 mmol). The mixture was stirred overnight. The volatile component was removed in vacuo, and the residue was partitioned between water and DCM. The aqueous layer was extracted with DCM. The combined organic phase was washed by brine, saturated sodium carbonate, and water, and dried over anhydrous Na₂SO₄. After concentration, the crude mixture was purified by flash column chromatography (Hex/Ethyl acetate=4/1 (v/v)) to afford (S)-2-(2-(6-bromonaphthalen-2-yl)-2-oxoethyl) 1-tert-butyl 4-methyl-1H-pyrrole-1,2(2H,5H)-dicarboxylate (1.2 g). LC-MS (ESI): m/z 496.2 [M+Na]⁺.

Step 2.

In a sealed tube, (S)-2-(2-(6-bromonaphthalen-2-yl)-2-oxoethyl) 1-tert-butyl 4-methyl-1H-pyrrole-1,2(2H,5H)-dicarboxylate (1.2 g, 2.53 mmol), ammonium acetate (2.92 g, 38 mmol) and triethylamine (0.7 mL, 5.06 mmol) were added in xylene (30 mL). The resulting mixture was stirred at 140° C. for 2 hrs. LC-MS showed the reaction was completed. The solvent was removed in vacuo, and the residue was partitioned between water and DCM. The aqueous layer was extracted with DCM. The combined organic phase was washed by brine, water, and dried over Na₂SO₄. After removing the solvents, the crude mixture was purified by flash column chromatography (Hex/EtOAc=1/1 (v/v)) to afford (S)-tert-butyl 2-(5-(6-bromonaphthalen-2-yl)-1H-imidazol-2-yl)-4-methyl-2,5-dihydro-1H-pyrrole-1-carboxylate (1-9b) (1.0 g). LC-MS (ESI): m/z 454.2 [M+H]⁺.

### (S)-tert-butyl 2-(5-(6-ethynylnaphthalen-2-yl)-1H-imidazol-2-yl)-4-methyl-2,5-dihydro-1H-pyrrole-1-carboxylate (1-10b)

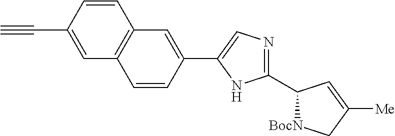 [SMILES: C#Cc1ccc2cc(-c3cnc([C@@H]4C=C(C)CN4C(=O)OC(C)(C)C)[nH]3)ccc2c1]

Step 1. General Procedure B.

To a solution of (S)-tert-butyl 2-(5-(6-bromonaphthalen-2-yl)-1H-imidazol-2-yl)-4-methyl-2,5-dihydro-1H-pyrrole-1-carboxylate (1-9b) (300 mg, 0.66 mmol) and trimethylsilylacetylene (0.44 mL, 3.09 mmol) in triethylamine (3 mL) was added copper iodide (8.3 mg) and Pd(PPh₃)₂Cl₂ (31 mg) at room temperature. After through degassing, the reaction was warmed up to 80° C. and stirred overnight while under nitrogen gas protection. The reaction was cooled to rt and diluted with ethyl acetate (100 mL) and washed with brine and water and then dried over anhydrous Na₂SO₄. After removal of solvents, the crude mixture was purified by flash column chromatography (Hexane/Ethyl acetate=2/1 (v/v)) to afford (S)-tert-butyl 4-methyl-2-(5-(6-((trimethylsilyl)ethynyl)naphthalen-2-yl)-1H-imidazol-2-yl)-2,5-dihydro-1H-pyrrole-1-carboxylate (230 mg). LC-MS (ESI): m/z 472.3 [M+H]⁺.

Step 2.

The mixture of the product from above (230 mg, 0.488 mmol) potassium carbonate (540 mg, 3.91 mmol) in methanol (6 mL) was warmed up to 80° C. and stirred overnight. The reaction was cooled to rt, diluted with ethyl acetate (100 mL) and washed with water and brine. The organic payer was dried over anhydrous Na₂SO₄, filtered, and concentrated to afford (S)-tert-butyl 2-(5-(6-ethynylnaphthalen-2-yl)-1H-imidazol-2-yl)-4-methyl-2,5-dihydro-1H-pyrrole-1-carboxylate (1-10b) (190 mg). LC-MS (ESI): m/z 400.30 [M+H]⁺.

### (S)-tert-butyl 2-(5-(6-bromonaphthalen-2-yl)-1H-imidazol-2-yl)-4-cyclopropyl-2,5-dihydro-1H-pyrrole-1-carboxylate (1-9d)

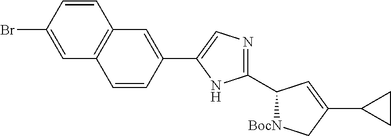 [SMILES: CC(C)(C)OC(=O)N1CC(C2CC2)=C[C@H]1c1ncc(-c2ccc3cc(Br)ccc3c2)[nH]1]

Prepared similarly as compound 1-9b, LC-MS (ESI): m/z 480 [M+H]⁺.

### (S)-tert-butyl 4-cyclopropyl-2-(5-(6-ethynylnaphthalen-2-yl)-1H-imidazol-2-yl)-2,5-dihydro-1H-pyrrole-1-carboxylate (1-10d)

LC-MS (ESI): m/z 426 [M+H]⁺.

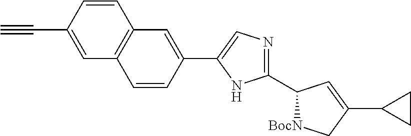 [SMILES: C#Cc1ccc2cc(-c3cnc([C@@H]4C=C(C5CC5)CN4C(=O)OC(C)(C)C)[nH]3)ccc2c1]

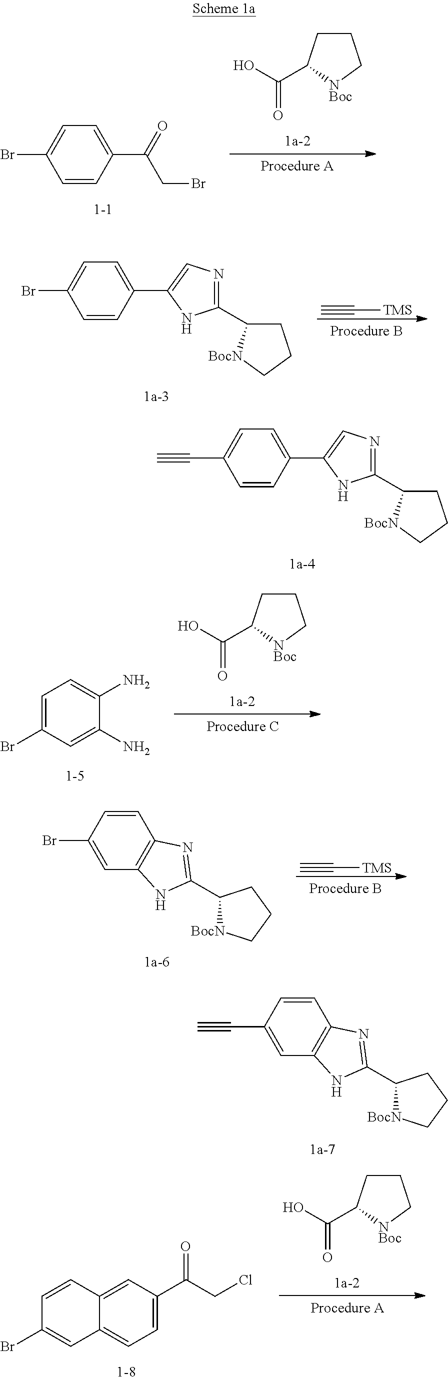

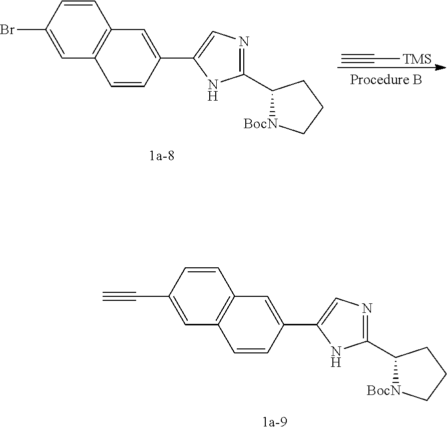

### (S)-tert-butyl 2-(5-(4-bromophenyl)-1H-imidazol-2-yl)pyrrolidine-1-carboxylate (1a-3)

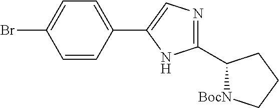 [SMILES: CC(C)(C)OC(=O)N1CCC[C@H]1c1ncc(-c2ccc(Br)cc2)[nH]1]

Referring to Scheme 1a, compound 1a-3 was prepared according to conditions described in General Procedure A. Step 1. To a solution of 2-bromo-1-(4-bromophenyl)ethanone 1-1 (120 g, 0.43 mol) in CH₃CN (300 mL) was added (S)—N-Boc-Pro-OH (97.0 g, 0.45 mol) and Et₃N (130 g, 1.29 mol), the mixture was stirred at room temperature for 2 h. The mixture was concentrated under reduced pressure to afford (S)-2-(2-(4-bromophenyl)-2-oxoethyl) 1-tert-butyl pyrrolidine-1,2-dicarboxylate. The crude product was used for next step without further purification.

Step 2.

To a solution of (S)-2-(2-(4-bromophenyl)-2-oxoethyl) 1-tert-butyl pyrrolidine-1,2-dicarboxylate (159 g, 0.39 mol) in xylene (250 mL) was added NH₄OAc (300 g, 3.90 mol), the mixture was stirred at 140° C. for overnight. The mixture was concentrated under reduced pressure, the residue was purified by silica gel column chromatography (petroleum ether/EtOAc=10/1 (v/v)) to afford (S)-tert-butyl 2-(4-(4-bromophenyl)-1H-imidazol-2-yl)pyrrolidine-1-carboxylate 1a-3 (105 g, 70% yield) as a white solid: ¹H NMR (500 MHz, CDCl₃): δ 1.48 (s, 9H), 1.96 (m, 1H), 2.16 (m, 2H), 3.01 (m, 1H), 3.42 (m, 2H), 4.96 (d, 1H, J=5.5 Hz), 7.22 (s, 1H), 7.46-7.55 (m, 4H) ppm; LCMS (ESI) m/z 392.1 (M+H)⁺.

Compound 1a-4, prepared according to General Procedure B, was obtained as a yellow solid in 80% yield in 2 steps from 1a-3. ¹H NMR (500 MHz, CDCl₃): δ 1.49 (s, 9H), 1.97 (m, 1H), 2.15 (m, 2H), 3.01 (brs, 1H), 3.40 (m, 2H), 4.96 (d, 1H, J=5.0 Hz), 7.24 (s, 1H), 7.47-7.52 (m, 4H) ppm; LC-MS (ESI): m/z 338 [M+H]⁺.

### (S)-tert-butyl 2-(6-ethynyl-1H-benzo[d]imidazol-2-yl)pyrrolidine-1-carboxylate (1a-6)

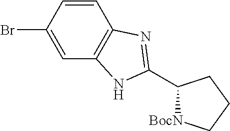 [SMILES: CC(C)(C)OC(=O)N1CCC[C@H]1c1nc2ccc(Br)cc2[nH]1]

Step 1. To a solution of N-Boc-L-Pro-OH (29 g, 135 mmol) and DIPEA (29 g, 225 mmol) in THF (500 mL) was added HATU (51 g, 135 mmol) at rt. After stirring at rt for 10 min, 4-bromobenzene-1,2-diamine (1-5) (25 g, 135 mmol) was added and the resulting solution was stirred at rt for another several hours. Subsequently, the reaction mixture was concentrated and the residue was diluted with EtOAc (500 mL). The resulting mixture was washed with water for several times (100 mL×3) and dried with anhydrous Na₂SO₄. The solvent was removed and the residue was dried in vacuo to give a mixture of acylated products, which were used for the next step without further purification.

Step 2.

A mixture of acylated products from above in AcOH (1000 mL) was stirred at 40° C. for 12 hrs. After cooling, the reaction mixture was carefully neutralized by adding saturated aqueous sodium bicarbonate solution to adjust the pH value to 8. The resulting mixture was extracted with EtOAc (250 mL×3). The combined extract was washed with water, and dried with anhydrous Na₂SO₄. The solvent was removed and the residue was purified by silica gel chromatography (Petroleum ether/EtOAc=4/1 (v/v)) to give (S)-tert-butyl 2-(6-bromo-1H-benzo[d]imidazol-2-yl)pyrrolidine-1-carboxylate (1a-6) (35 g, 71% yield in 2 steps) as a yellow solid. LC-MS (ESI): m/z 366.1 [M+H]⁺.

### (S)-tert-butyl 2-(6-ethynyl-1H-benzo[d]imidazol-2-yl)pyrrolidine-1-carboxylate (1a-7)

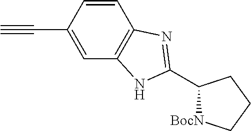 [SMILES: C#Cc1ccc2nc([C@@H]3CCCN3C(=O)OC(C)(C)C)[nH]c2c1]

Compound 1a-7 was prepared following General Procedure B, and was obtained in 75% overall yield for 2 steps. LC-MS (ESI): m/z 312.2 [M+H]⁺.

### (S)-tert-butyl 2-(5-(6-bromonaphthalen-2-yl)-1H-imidazol-2-yl)pyrrolidine-1-carboxylate (1a-8)

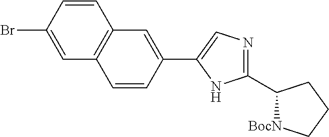 [SMILES: CC(C)(C)OC(=O)N1CCC[C@H]1c1ncc(-c2ccc3cc(Br)ccc3c2)[nH]1]

Step 1.

To a solution of 2-bromonaphthalene (1-7) (62 g, 0.3 mol) in DCM (1000 mL) was added AlCl₃ (44 g, 0.33 mol), followed by 2-chloroacetyl chloride (34 g, 0.3 mmol) at 0° C. After stirring at 0° C. for 1 hr, the reaction mixture was quenched by adding water (500 mL). The organic layer was separated, washed with brine, and dried with anhydrous Na₂SO₄. The solvent was removed and the residue was re-crystallized in 10% of EtOAc in hexane to give compound 1-8 (28 g, 33% yield) as a white solid. ¹H NMR (500 MHz, CDCl₃): δ 8.44 (s, 1H), 8.07 (s, 1H), 8.04 (d, J=11.0 Hz, 1H), 7.84 (d, J=8.5 Hz, 2H), 7.66 (d, J=8.5 Hz, 1H), 4.81 (s, 2H) ppm; LC-MS (ESI): m/z 282.9 [M+H]

Starting from 1-(6-bromonaphthalen-2-yl)-2-chloroethanone (1-8) (28 g, 99 mmol) and following General Procedure A, compound 1a-8 was obtained as a yellow solid (30 g, 68% yield). LC-MS (ESI): m/z 442.1 [M+H]⁺.

### (S)-tert-butyl 2-(5-(6-ethynylnaphthalen-2-yl)-1H-imidazol-2-yl)pyrrolidine-1-carboxylate (1a-9)

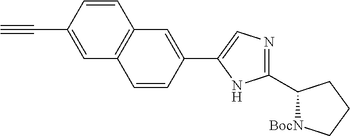 [SMILES: C#Cc1ccc2cc(-c3cnc([C@@H]4CCCN4C(=O)OC(C)(C)C)[nH]3)ccc2c1]

Treatment of compound 1a-8 under the conditions described in General Procedure B yielded compound 1a-9 (1.3 g, 77% yield) as a yellow solid. LC-MS (ESI): m/z 388.2 [M+H]⁺.

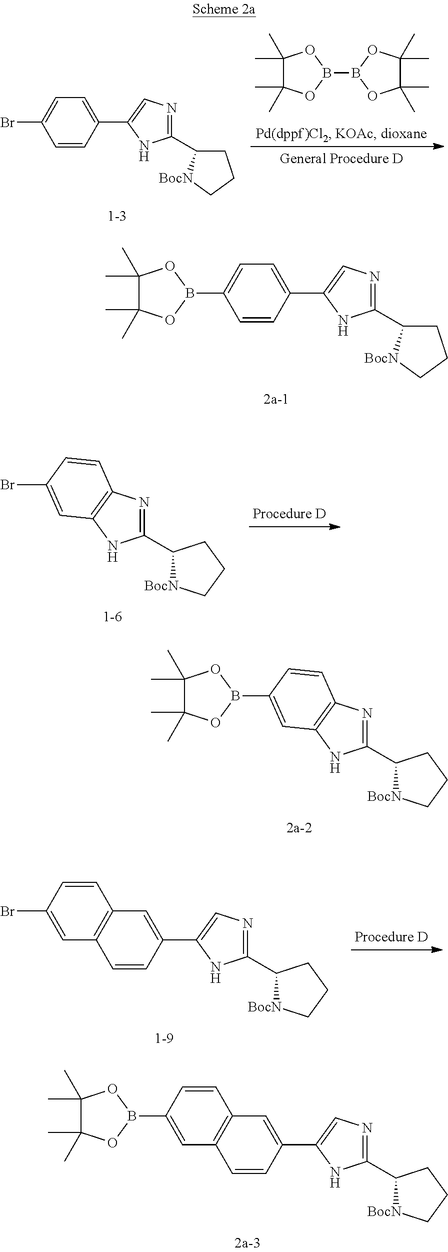

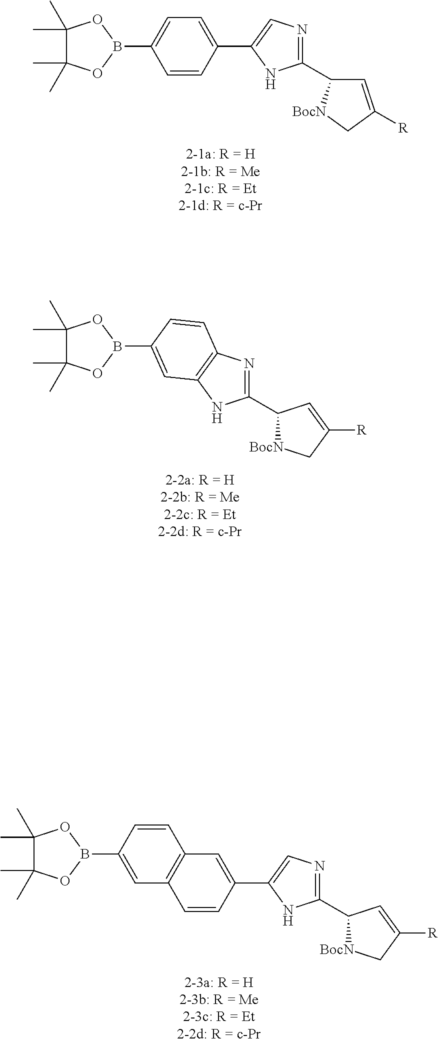

### (S)-tert-butyl 2-(5-(4-(4,4,5,5-tetramethyl-1,3,2-dioxaborolan-2-yl)phenyl)-1H-imidazol-2-yl)pyrrolidine-1-carboxylate (2a-1)

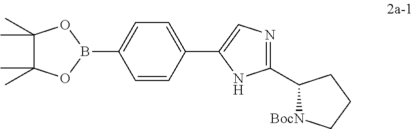 [SMILES: CC(C)(C)OC(=O)N1CCC[C@H]1c1ncc(-c2ccc(B3OC(C)(C)C(C)(C)O3)cc2)[nH]1]

General Procedure D:

preparation of arylboronate (or boronic acid) from aryl halide by a Suzuki Reaction. To a mixture of 1a-3 (4.90 g, 12.5 mmol), bis(pinacolato)diboron (7.10 g, 26.3 mmol), potassium acetate (3.20 g, 32.5 mmol) in 1,4-dioxane (100 mL) was added Pd[dppf]Cl₂ (400 mg, 0.500 mmol). After stirring at 80° C. for 3 hrs, the reaction mixture was filtered and concentrated in vacuo. The residue was purified with silica gel column chromatography (Petroleum ether/EtOAc=2/1 (v/v)) to provide 2a-1 (3.0 g, 53% yield) as a gray solid: LC-MS (ESI) m/z 440 [M+H]⁺.

### (S)-tert-butyl 2-(6-(4,4,5,5-tetramethyl-1,3,2-dioxaborolan-2-yl)-1H-benzo[d]imidazol-2-yl)pyrrolidine-1-carboxylate (2a-2)

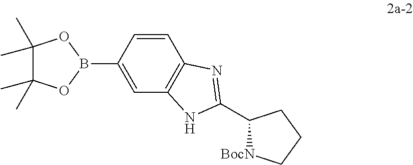 [SMILES: CC(C)(C)OC(=O)N1CCC[C@H]1c1nc2ccc(B3OC(C)(C)C(C)(C)O3)cc2[nH]1]

Compound 2a-2 was prepared from 1a-6 by following General Procedure D. (3.3 g, 58% yield). LC-MS (ESI): m/z 414.2 [M+H]⁺.

### (S)-tert-butyl 2-(5-(6-(4,4,5,5-tetramethyl-1,3,2-dioxaborolan-2-yl)naphthalen-2-yl)-1H-imidazol-2-yl)pyrrolidine-1-carboxylate (2a-3)

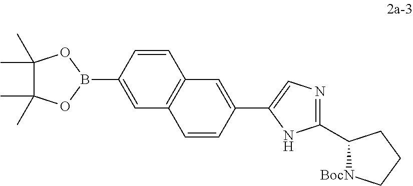 [SMILES: CC(C)(C)OC(=O)N1CCC[C@H]1c1ncc(-c2ccc3cc(B4OC(C)(C)C(C)(C)O4)ccc3c2)[nH]1]

Compound 2a-3 was prepared from 1a-8 by following General Procedure D. LC-MS (ESI): m/z 490.3 [M+H]⁺.

Other boronate building blocks represented by 2-1a to 2-1d, 2-2a to 2-2d, and 2-3a to 3-3d may be prepared similarly.

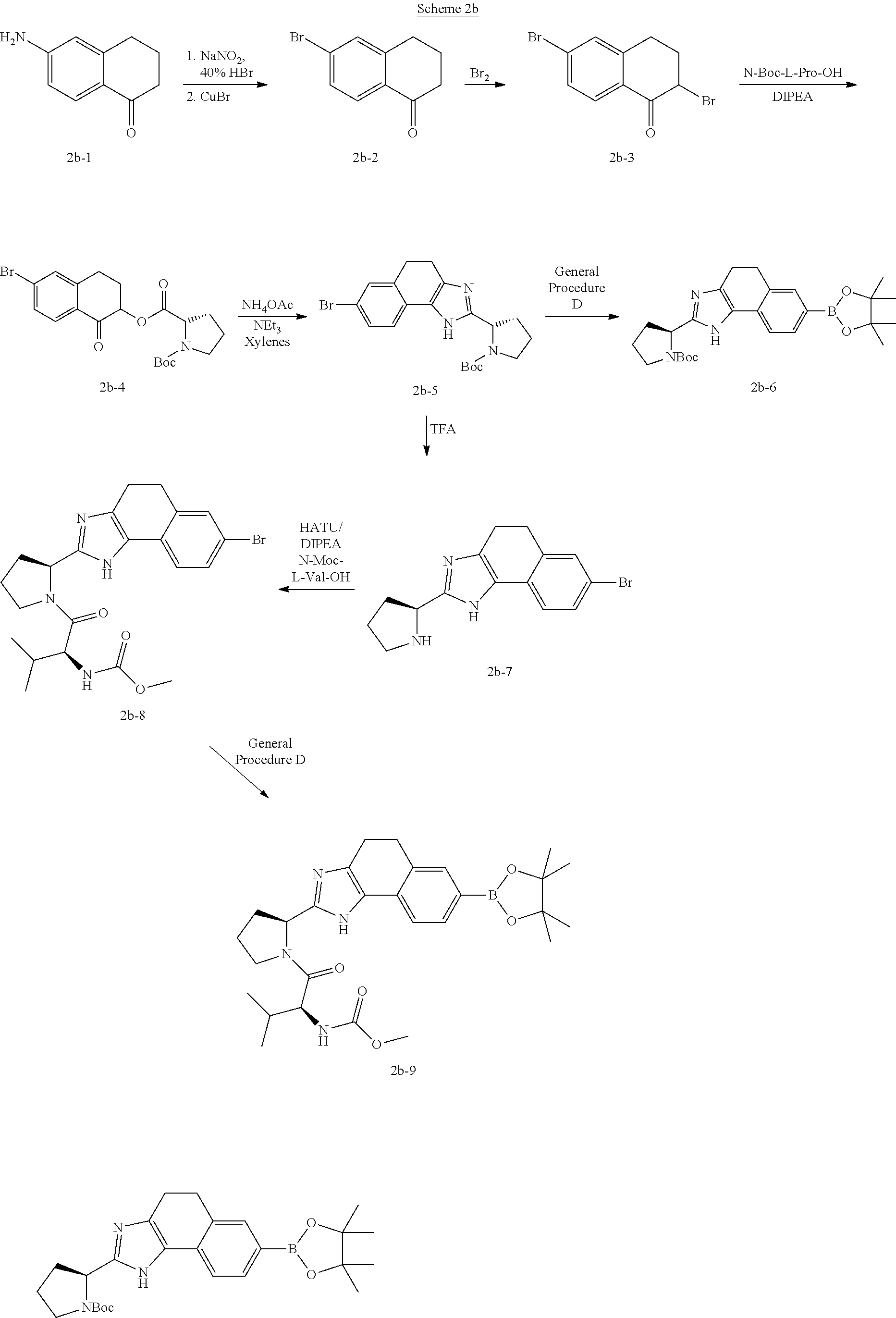

### (S)-tert-butyl 2-(7-bromo-4,5-dihydro-1H-naphtho[2,1-d]imidazol-2-yl)pyrrolidine-1-carboxylate (2b-3)

Step 1.

Referring to Scheme 2b, to a solution of 2b-1 (20.6 g, 0.128 mol) in 45 mL of 48% hydrobromic acid and 10 mL of water was added a solution of 9.72 g (0.141 mol) of sodium nitrite in 18 mL of water, maintaining a temperature below 5° C. After stirring at 5° C. for 1 hr, CuBr (0.128 mol) was added and the resulting mixture was stirred at rt for 3 hrs. Subsequently, the mixture was extracted with EtOAc (2×200 mL). The extracts were combined, washed with brine, and dried with anhydrous Na₂SO₄. The solvent was removed and the residue was purified by silica gel column chromatography (Hex/EtOAc=12/1 (v/v)) to afford 2b-2 (13.3 g, 46% yield) as a powder. ¹H NMR (CDCl₃, 400 MHz): δ 7.90 (d, 1H), 7.44 (m, 2H), 2.96 (t, 2H), 2.64 (t, 2H), 2.15 (m, 2H) ppm.

Step 2.

To a solution of 2b-2 (12.49 g, 55.5 mmol) in 300 mL of methylene chloride and 0.30 mL of 48% hydrobromic acid was slowly added 3.1 mL of bromine at 0° C. The reaction mixture was gradually warmed up to rt, and kept stirring for another 2 hrs. The organic solution was washed with saturated NaHCO₃ twice, and then with water. The crude product was purified by silica gel column chromatography to afford 2b-3 (11.9 g, 71% yield). ¹H NMR (CDCl₃, 400 MHz): δ 7.94 (d, 2H), 7.52 (m, 2H), 4.72 (t, 1H), 3.32 (m, 1H), 2.92 (m, 1H), 2.48 (m, 2H) ppm.

Step 3.

A mixture of 2b-3 (11.80 g, 38.8 mmol), N-Boc-L-Pro-OH (10.02 g, 46.6 mmol), and diisopropylethylamine (7.02 g, 54.3 mmol) in acetonitrile (200 mL) was stirred at 50° C. for 10 hrs. The solvent was evaporated and the residue was partitioned between methylene chloride and water. The organic layer was separated and concentrated to dryness. The crude product was purified by silica gel column chromatography (hexanes/ethyl acetate=1/7 to 1/4 (v/v)) to provide 2b-4 (11.53 g, 68% yield) as a white solid. ¹H NMR (CDCl₃, 400 MHz): δ 7.84 (m, 1H), 7.48 (m, 2H), 5.58 (m, 1H), 4.40 (m, 1H), 3.60 (m, 1H), 3.40 (m, 1H), 3.18 (m, 1H), 3.04 (m, 1H), 2.37 (m, 2H), 2.04 (m, 1H), 1.96 (m, 1H), 1.46 (ds, 9H) ppm.

Step 4.

A mixture of 2b-4 (11.09 g, 25.3 mmol), ammonium acetate (29.25 g, 38.0 mmol) and triethylamine (38.45 g, 38.0 mmol) in xylenes (600 mL) in a sealed tube was stirred at 140° C. for 2 hrs. After being cooled, the reaction mixture was transferred into a flask and concentrated to dryness. The residue was partitioned between chloroform and water, and the organic layer was washed with water, and concentrated. The crude product was purified by silica gel column chromatography (NH₄OH/ACN/EtOAc=1/8/100 (v/v/v)) to afford 2b-5 (8.22 g, 75% yield) as a white solid. LC-MS (ESI): m/z 420.1 [M+H]⁺.

### Step 5. (S)-tert-butyl 2-(7-(4,4,5,5-tetramethyl-1,3,2-dioxaborolan-2-yl)-4,5-dihydro-1H-naphtho[1,2-d]imidazol-2-yl)pyrrolidine-1-carboxylate (2b-6).

Compound 2b-6 was prepared from 2b-5 using the conditions described in General Procedure D.

Step 6. General Procedure G:

N-Boc deprotection and reacylation (Step 6 and 7). Trifluoroacetic acid (20 mL) was slowly added into a solution of 2b-5 (4.80 g, 11.4 mmol) in methylene chloride (40 mL) at rt. After stirring at rt for 2 hrs, the reaction mixture was concentrated and the residue was dried in vacuo to give a TFA salt 2b-7, which was used for the next step without further purification. LC-MS (ESI): m/z 318.1 [M+H]⁺.

Step 7.

To a mixture of the TFA salt 2b-7 (6.28 g, 11.5 mmol) in DMF (23 mL) was added DIPEA (22.8 mL, 138 mmol), followed by N-Moc-L-Val-OH (2.42 g, 13.8 mmol) and HATU (5.25 g, 13.8 mmol). After stirring at rt for 2 hrs, the reaction mixture was slowly dropped into water while stirring. The resulting precipitate was collected by filtration. The crude product was purified by silica gel column chromatography (Hex/EtOAc=1/4 (v/v) to pure EtOAc) to afford 2b-8 (4.43 g, 81% yield). LC-MS (ESI): m/z 475.3 [M+H]⁺.

Step 8.

To a mixture of compound 2b-8 (2.5 g, 5.27 mmol), bis(pinacolato)diboron (2.6 g, 10.5 mmol), potassium acetate (2.2 g, 15.8 mmol) in 1,4-dioxane (50 mL) was added Pd[dppf]Cl₂ (260 mg, 0.3 mmol) at rt under an atmosphere of N₂. After stirring at 80° C. for 3 hrs under an atmosphere of N₂, the reaction mixture was filtered through Celite™ 545 and the filter cake was washed with EtOAc for several times (30 mL×3). The filtrate was washed with brine and dried with anhydrous Na₂SO₄. The solvent was removed and the residue was purified by silica gel column chromatography (Petroleum ether/EtOAc=2/1 (v/v)) to give compound 2b-9 (1.6 g, 58% yield). LC-MS (ESI): m/z 522.3 [M+H]⁺.

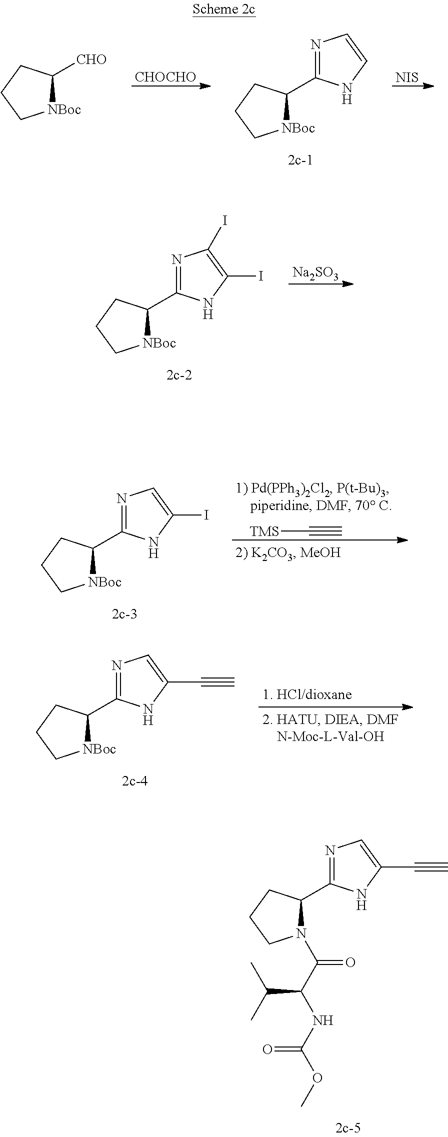

### (S)-tert-butyl 2-(5-iodo-1H-imidazol-2-yl)pyrrolidine-1-carboxylate (2c-3)

Referring to Scheme 2c, Step 1. To a solution of freshly prepared N-Boc-L-prolinaldehyde (20.0 g, 0.10 mol) in MeOH (200 mL) was added glyoxal (20.0 g, 0.34 mol) and NH₄OH (68.0 g, 1.90 mol), the mixture was stirred at rt overnight. The organic solvent was removed under reduced pressure and the residue was purified by silica gel column chromatography (PE/EtOAc=1/1 (v/v)) to afford (S)-tert-butyl 2-(1H-imidazol-2-yl)pyrrolidine-1-carboxylate (10.7 g, 45% yield) as a white solid. ¹H NMR (500 MHz, CDCl₃): δ 1.48 (s, 9H), 1.96-2.12 (m, 3H), 2.91-2.92 (m, 1H), 3.38 (m, 2H), 4.93 (d, 1H, J=7.0 Hz), 6.96 (s, 2H) ppm. LC-MS (ESI): m/z 238.2 [M+H]⁺.

Step 2.

To a solution of (S)-tert-butyl 2-(1H-imidazol-2-yl)pyrrolidine-1-carboxylate (2c-1) (10.0 g, 42.2 mmol) in DCM (300 mL) was added NIS (19.0 g, 84.4 mmol) slowly at 0° C., the reaction mixture was stirred for 1 hr at this temperature. The organic solvent was removed and the residue purified by silica gel column chromatography (Petroleum ether/EtOAc=3/1 (v/v)) to afford (S)-tert-butyl 2-(4,5-diiodo-1H-imidazol-2-yl)pyrrolidine-1-carboxylate (18.2 g, 88% yield) as a yellow solid. LC-MS (ESI): m/z 490 [M+H]⁺.

Step 3.

To a suspension of (S)-tert-butyl 2-(4,5-diiodo-1H-imidazol-2-yl)pyrrolidine-1-carboxylate (2c-2) (18.0 g, 36.8 mmol) in 800 mL EtOH/H₂O (v/v=30:70) solution was added Na₂SO₃ (39.4 g, 312.9 mmol), the mixture was refluxed for 17 hrs. EtOH was evaporated under reduced pressure, and the residue was diluted with EtOAc, the organic layer was washed with brine and dried over Na₂SO₄, then concentrated to dryness, the residue was purified by silica gel column chromatography (Petroleum ether/EtOAc=3/1 (v/v)) to afford (S)-tert-butyl 2-(4-iodo-1H-imidazol-2-yl)pyrrolidine-1-carboxylate (2c-3) (10.5 g, 80% yield) as a white solid. ¹H NMR (500 MHz, DMSO): δ 1.16 (s, 5H), 1.38 (s, 4H), 1.80-1.91 (m, 3H), 2.08-2.18 (m, 1H), 3.30-3.46 (m, 2H), 4.66-4.76 (m, 1H), 7.16 (d, 1H, J=14 Hz), 12.04-12.09 (m, 1H) ppm; LC-MS (ESI): m/z 364.0 [M+H]⁺.

### (S)-tert-butyl 2-(5-ethynyl-1H-imidazol-2-yl)pyrrolidine-1-carboxylate (2c-4)

Step 1.

A mixture of compound 2c-3 (54.5 g, 0.15 mol), trimethylsilylacetylene (17.7 g, 0.18 mol), P(t-Bu)₃ (121.4 g, 0.6 mol), piperidine (51.0 g, 0.6 mol), and Pd[PPh₃]₂Cl₂ (10.5 g, 15 mmol) in DMF (300 mL) was stirred at 70° C. overnight under an atmosphere of N₂. Subsequently, the reaction mixture was concentrated and the residue was diluted with EtOAc (500 mL). The resulting mixture was washed with water for several times (100 mL×3) and dried with anhydrous Na₂SO₄. The solvent was removed and the residue was purified by silica gel column chromatography to give the TMS-acetylene compound (27.5 g, 55% yield). LC-MS (ESI): m/z 334.2 [M+H]⁺.

Step 2.

A mixture of the TMS-acetylyene product obtained from the above reaction (25 g, 75 mmol) and K₂CO₃ (41.5 g, 300 mmol) in MeOH (250 mL) and THF (250 mL) was stirred at rt for 2 hrs. Subsequently, the reaction mixture was filtered through pad of Celite° 545 and the filter cake was washed with EtOAc several times (100 mL×3). The filtrate was concentrated and the residue was diluted with EtOAc (500 mL). The resulting mixture was washed with water for several times (100 mL×3) and dried with anhydrous Na₂SO₄. The solvent was removed and the residue was purified by silica gel column chromatography to give (S)-tert-butyl 2-(5-ethynyl-1H-imidazol-2-yl)pyrrolidine-1-carboxylate (2c-4) (12.3 g, 63% yield). LC-MS (ESI): m/z 262.1 [M+H]⁺.

### Methyl (S)-1-((S)-2-(5-ethynyl-1H-imidazol-2-yl)pyrrolidin-1-yl)-3-methyl-1-oxobutan-2-ylcarbamate (2c-5)

General Procedure G.

A mixture of compound 2c-4 (10 g, 38.3 mmol) in 4 N HCl/dioxane (100 mL) was stirred at rt for 2 hrs. The reaction mixture was concentrated and the residue was dried in vacuo to give an HCl salt, which was used for the next step without further purification. LC-MS (ESI): m/z 162.1 [M+H]⁺.

Subsequently, the HCl salt was dissolved in DMF (120 mL) and the resulting mixture was sequentially added Et₃N (19.3 g, 191 mmol), N-Moc-L-Val-OH (7.4 g, 42 mmol), and HATU (16 g, 42 mmol). After stirring at rt for 1 hr, the reaction mixture was concentrated and the residue was diluted with DCM (150 mL). The resulting mixture was washed with water several times (100 mL×3) and dried with anhydrous Na₂SO₄. The solvent was removed and the residue was purified by silica gel column chromatography (DCM/EtOAc=4/1 (v/v)) to give compound 2c-5 (7.0 g, 57% yield). LC-MS (ESI): m/z 319.2 [M+H]⁺.

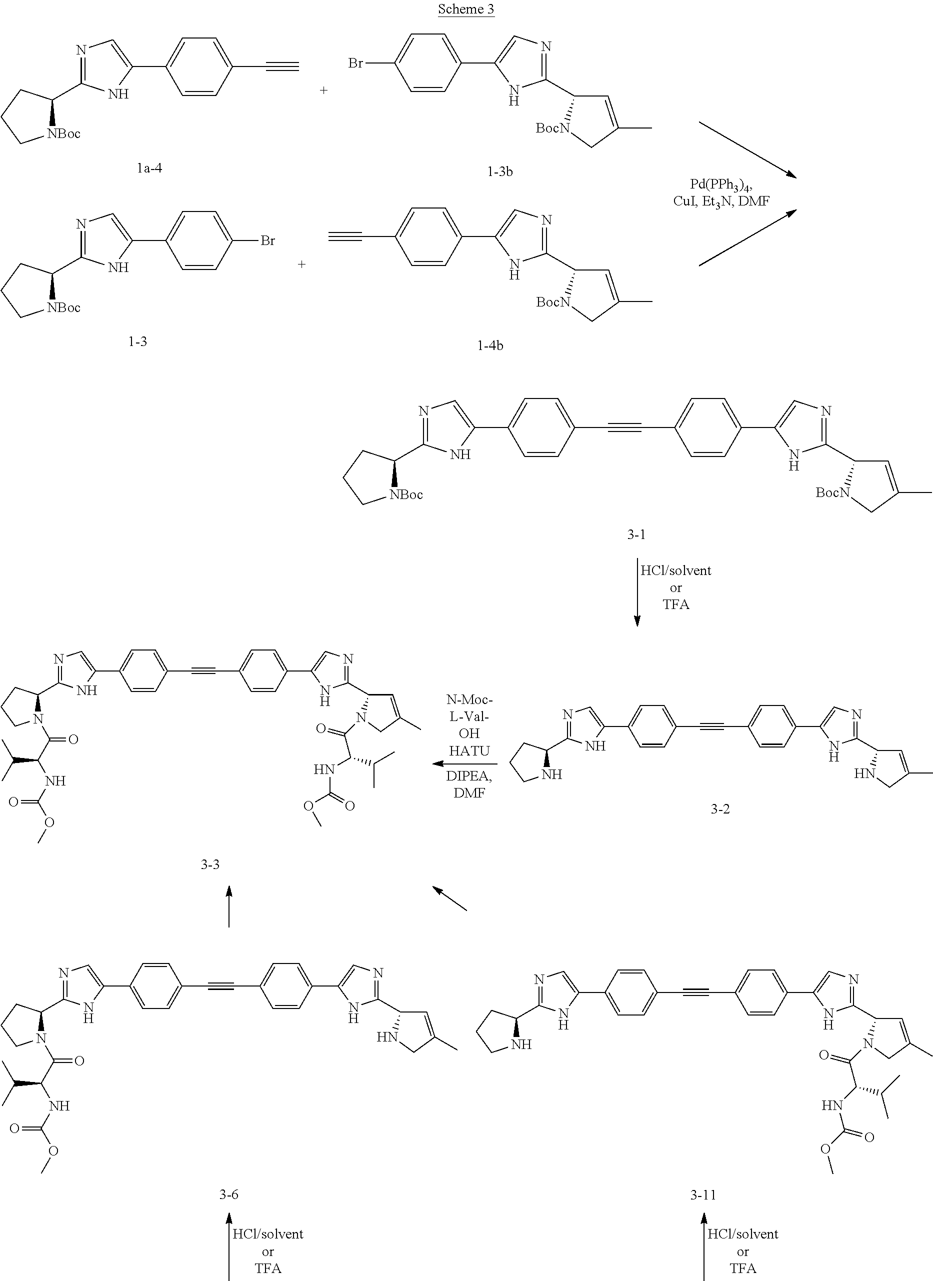

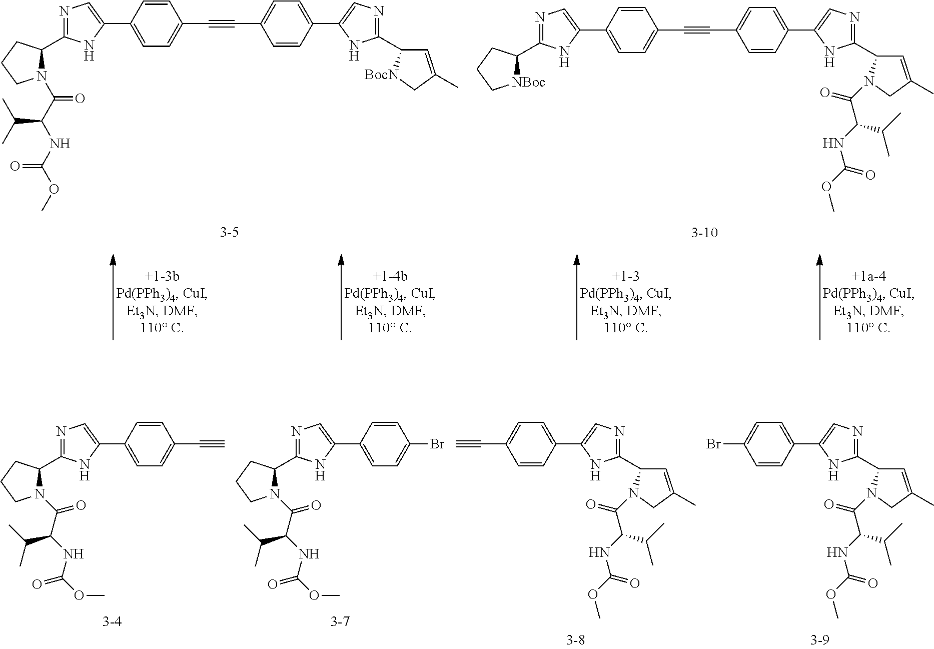

General Procedure E:

Sonogashira cross coupling of an arylacetylene and an arylhalide. To a solution of 1-4b (200 mg, 0.50 mmol) in DMF (6.0 mL) in a sealed tube was added 1a-4 (187 mg, 0.55 mmol), tetrakis(triphenylphosphine)palladium (58 mg, 0.05 mmol), CuI (19 mg, 0.1 mmol) and triethylamine (0.2 mL, 1.5 mmol). The resulting solution was degassed and heated at 110° C. overnight. After cooled to rt, the reaction mixture was partitioned between water and DCM. The aqueous layer was extracted with DCM. The combined organic layer was dried with anhydrous Na₂SO₄, filtered, and concentrated. The crude mixture was purified by flash column chromatography (EtOAc/ACN/NH₄OH=100/7/1 (v/v/v)) to afford (S)-tert-butyl 2-(5-(4-((4-(2-((S)-1-(tert-butoxycarbonyl)pyrrolidin-2-yl)-1H-imidazol-5-yl)phenyl)ethynyl)phenyl)-1H-imidazol-2-yl)-4-methyl-2,5-dihydro-1H-pyrrole-1-carboxylate (3-1) (150 mg, 47% yield). LC-MS (ESI): m/z 661 [M+H]⁺.

Compound 3-1 is alternatively obtained by reacting 1a-4 and 1-4b under the same set of conditions described in General Procedure E.

### Methyl N-[(2S)-1-[(2S)-2-(5-{4-[2-(4-{2-[(2S)-1-[(2S)-2-[(methoxycarbonyl)amino]-3-methylbutanoyl]-4-methyl-2,5-dihydro-1H-pyrrol-2-yl]-1H-imidazol-5-yl}phenyl)ethynyl]phenyl}-1H-imidazol-2-yl)pyrrolidin-1-yl]-3-methyl-1-oxobutan-2-yl]carbamate (3-3).

Compound 3-3 was obtained via compound 3-2 following General Procedure G described previously. LC-MS (ESI): m/z 775.4 [M+H]⁺.

One of the alternative routes to prepare compound 3-3 was outlined Scheme 3, via 3-4, 3-5, and 3-6. More specifically, compound 3-4 was obtained by treating a sample of compound 1a-4 under the procedures described in General Procedure G.

Under General Procedure E described previously, to a solution of 1-3b (200 mg, 0.50 mmol) in DMF (6.0 mL) in a sealed tube was added 3-4 (187 mg, 0.6 mmol), tetrakis(triphenylphosphine)palladium (58 mg, 0.05 mmol), CuI (19 mg, 0.1 mmol) and triethylamine (0.2 mL, 1.5 mmol). The resulting solution was degassed, sealed and heated at 110° C. overnight. The reaction mixture was cooled to rt, and then partitioned between water and DCM. The aqueous layer was extracted with DCM. The combined organic layer was dried with anhydrous Na₂SO₄, filtered, and concentrated. The crude mixture was purified by flash column chromatography (EtOAc/ACN/NH₄OH=100/7/1 (v/v/v)) to afford compound 3-5 (150 mg, 47% yield). LC-MS (ESI): m/z 718.4 [M+H]⁺.

Treatment of a sample of 3-5 under the conditions detailed in General Procedure G, compound 3-3 was obtained via intermediate 3-6.

Those skilled in the art will understand that analogs of compound 3-3 in which the dihydropyrrole moiety is functionalized with different amino acid residues (in addition to the substituted valine shown) can be readily prepared by reacting intermediate 3-6 with the chosen amino acids under standard peptide coupling conditions. Applying the procedures and conditions described in the above examples, analogs of 3-3 which the pyrrolidine and the dihydropyrrole moieties are substituted by other ring structures may be obtained, such as compounds 3-12 and 3-13 etc.

### Methyl N-[(2S)-1-[(2S)-2-(5-{4-[2-(4-{2-[(2S)-4-cyclopropyl-1-[(2S)-2-[(methoxycarbonyl)amino]-3-methylbutanoyl]-2,5-dihydro-1H-pyrrol-2-yl]-1H-imidazol-5-yl}phenyl)ethynyl]phenyl}-1H-imidazol-2-yl)pyrrolidin-1-yl]-3-methyl-1-oxobutan-2-yl]carbamate (3-12).

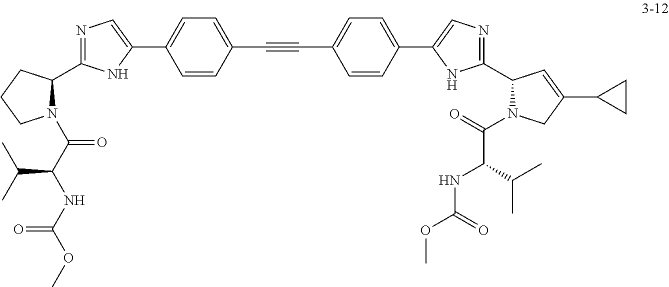 [SMILES: COC(=O)N[C@H](C(=O)N1CC(C2CC2)=C[C@H]1c1ncc(-c2ccc(C#Cc3ccc(-c4cnc([C@@H]5CCCN5C(=O)[C@@H](NC(=O)OC)C(C)C)[nH]4)cc3)cc2)[nH]1)C(C)C]

LC-MS (ESI): m/z 801.4 [M+H]⁺.

### Methyl N-[(2S)-1-[(2S)-2-(5-{4-[2-(4-{2-[(2S)-1-[(2S)-2-[(methoxycarbonyl)amino]-3-methylbutanoyl]-4-methyl-2,5-dihydro-1H-pyrrol-2-yl]-1H-imidazol-5-yl}phenyl)ethynyl]phenyl}-1H-imidazol-2-yl)-4-methyl-2,5-dihydro-1H-pyrrol-1-yl]-3-methyl-1-oxobutan-2-yl]carbamate (3-13)

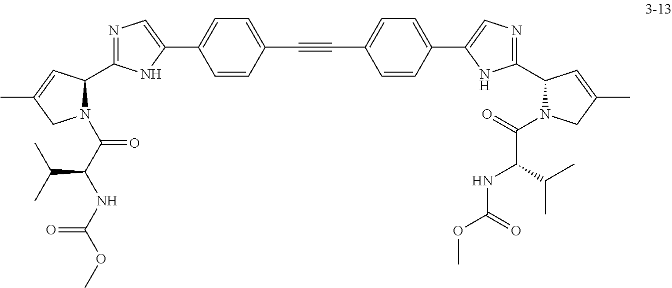

LC-MS (ESI): m/z 787.4 [M+H]⁺.

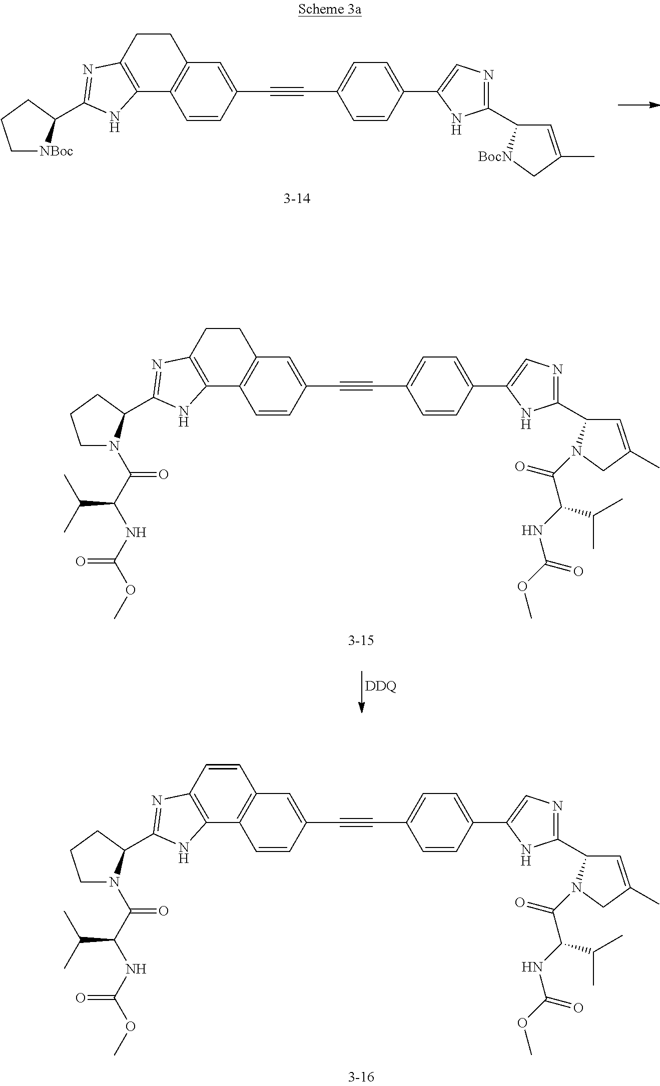

### Methyl N-[(2S)-1-[(2S)-2-{7-[2-(4-{2-[(2S)-1-[(2S)-2-[(methoxycarbonyl)amino]-3-methylbutanoyl]-4-methyl-2,5-dihydro-1H-pyrrol-2-yl]-1H-imidazol-5-yl}phenyl)ethynyl]-1H,4H,5H-naphtho[1,2-d]imidazol-2-yl}pyrrolidin-1-yl]-3-methyl-1-oxobutan-2-yl]carbamate (3-11)

Referring to the synthetic Scheme 3a, a Sonogoshira coupling reaction between compound 1-4b and compound 2b-5 under the conditions described in General Procedure B led to 3-14, which in turn was converted to compound 3-15 under the same procedures and conditions described in General Procedure G. LC-MS (ESI): m/z 801.4 [M+H]⁺.

### Methyl N-[(2S)-1-[(2S)-2-{7-[2-(4-{2-[(2S)-1-[(2S)-2-[(methoxycarbonyl)amino]-3-methylbutanoyl]-4-methyl-2,5-dihydro-1H-pyrrol-2-yl]-1H-imidazol-5-yl}phenyl)ethynyl]-1H-naphtho[1,2-d]imidazol-2-yl}pyrrolidin-1-yl]-3-methyl-1-oxobutan-2-yl]carbamate (3-16)

Compound 3-15 was readily converted by treating with DDQ to 3-16. LC-MS (ESI): m/z 799.4 [M+H]⁺.

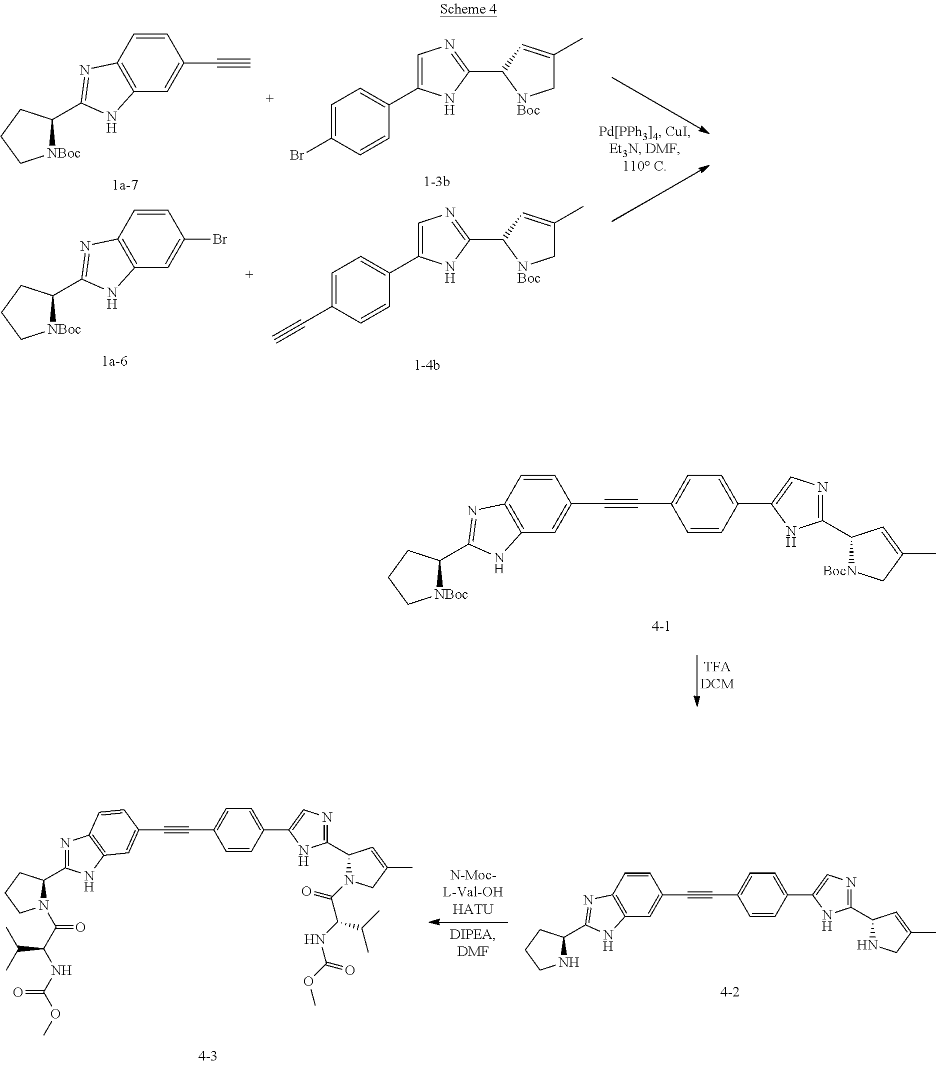

### (S)-tert-butyl 2-(5-(4-((2-((S)-1-(tert-butoxycarbonyl)pyrrolidin-2-yl)-1H-benzo[d]imidazol-6-yl)ethynyl)phenyl)-1H-imidazol-2-yl)-4-methyl-2,5-dihydro-1H-pyrrole-1-carboxylate (4-1)

General Procedure E

Sonogashira coupling of an arylacetylene and an arylhalide. To a solution of 1-3b (200 mg, 0.50 mmol) in DMF (6.0 mL) in a sealed tube was added 1a-7 (187 mg, 0.6 mmol), tetrakis(triphenylphosphine)palladium (58 mg, 0.05 mmol), CuI (19 mg, 0.1 mmol) and triethylamine (0.2 mL, 1.5 mmol). The resulting solution was degassed, sealed and heated at 110° C. overnight. After cooled to rt, the reaction mixture was partitioned between water and DCM. The aqueous layer was extracted with DCM. The combined organic phases were dried with anhydrous Na₂SO₄, filtered, and concentrated. The crude mixture was purified by flash column chromatography (EtOAc/Acetonitrile/NH₄OH=100/7/1 (v/v/v)) to afford compound 4-1 (150 mg, 47% yield). LCMS (ESI): m/z 635 [M+H]⁺.

As outlined in Scheme 4, compound 4-1 is alternatively prepared by reacting 1a-6 and 1-4b under the conditions described in General Procedure E.

### 6-((4-(2-((S)-4-methyl-2,5-dihydro-1H-pyrrol-2-yl)-1H-imidazol-5-yl)phenyl)ethynyl)-2-((S)-pyrrolidin-2-yl)-1H-benzo[d]imidazole (4-2)

To a solution of 4-1 (150 mg, 0.24 mmol) in DCM (3 mL) was added TFA (1.5 mL). The resulting solution was stirred at rt for 2 hrs. The solvent and reagent were removed. The Boc-deprotected product 4-2 was used in the next acylation step.

### Methyl N-[(2S)-1-[(2S)-2-{6-[2-(4-{2-[(2S)-1-[(2S)-2-[(methoxycarbonyl)amino]-3-methylbutanoyl]-4-methyl-2,5-dihydro-1H-pyrrol-2-yl]-1H-imidazol-5-yl}phenyl)ethynyl]-1H-1,3-benzodiazol-2-yl}pyrrolidin-1-yl]-3-methyl-1-oxobutan-2-yl]carbamate (4-3)

To a solution of N-Moc-L-Val-OH (91 mg, 0.29 mmol) in DMF (2 mL) was added HATU (269 mg, 0.71 mmoL) and DIPEA (0.47 mL, 2.83 mmol). The resulting mixture was stirred at rt for 20 min, then transferred into a solution of 4-2 (0.24 mmol) in DMF (2 mL). The mixture was stirred at rt for another 2 hrs. The reaction mixture was partitioned between water and DCM. The aqueous layer was extracted with DCM. The combined organic phase was dried with anhydrous Na₂SO₄, filtered, and concentrated. The crude mixture was purified by prep HPLC to afford 4-3 (40 mg). ¹H NMR (300 MHz, CD₃OD): δ 7.64 (m, 2H), 7.48 (m, 3H), 7.37 (m, 2H), 7.06 (dd, 1H), 5.82 (s, 1H), 5.51 (s, 1H), 5.13 (t, 1H), 4.63 (dd, 2H), 4.25 (t, 1H), 4.14 (t, 1H), 4.04 (m, 1H), 3.92 (m, 1H), 3.62 (s, 6H), 2.42-2.25 (m, 2H), 2.21-2.16 (m, 1H), 2.10-1.98 (m, 3H), 1.92 (s, 3H), 0.98-0.82 (m, 12H) ppm. LC-MS (ESI): m/z 749.6 [M+H]⁺, 375.5 (M+2)/2²⁺, 747.5 [M−H]⁻.

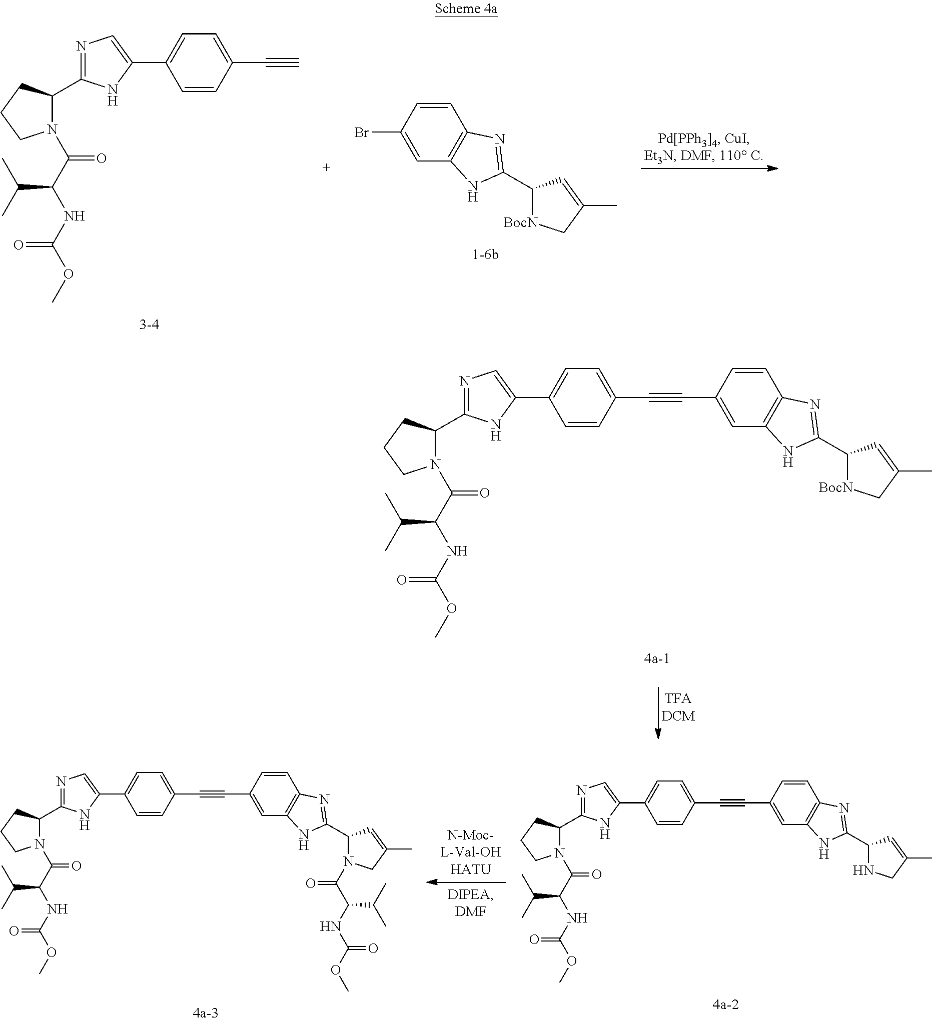 [SMILES: C#Cc1ccc(-c2cnc([C@@H]3CCCN3C(=O)[C@@H](NC(=O)OC)C(C)C)[nH]2)cc1 ; CC1=C[C@@H](c2nc3ccc(Br)cc3[nH]2)N(C(=O)OC(C)(C)C)C1 ; COC(=O)N[C@H](C(=O)N1CCC[C@H]1c1ncc(-c2ccc(C#Cc3ccc4nc([C@@H]5C=C(C)CN5C(=O)OC(C)(C)C)[nH]c4c3)cc2)[nH]1)C(C)C ; COC(=O)N[C@H](C(=O)N1CC(C)=C[C@H]1c1nc2ccc(C#Cc3ccc(-c4cnc([C@@H]5CCCN5C(=O)[C@@H](NC(=O)OC)C(C)C)[nH]4)cc3)cc2[nH]1)C(C)C ; COC(=O)N[C@H](C(=O)N1CCC[C@H]1c1ncc(-c2ccc(C#Cc3ccc4nc([C@@H]5C=C(C)CN5)[nH]c4c3)cc2)[nH]1)C(C)C]

### (S)-tert-butyl 2-(6-((4-(2-((S)-1-((S)-2-(methoxycarbonylamino)-3-methylbutanoyl)pyrrolidin-2-yl)-1H-imidazol-5-yl)phenyl)ethynyl)-1H-benzo[d]imidazol-2-yl)-4-methyl-2,5-dihydro-1H-pyrrole-1-carboxylate (4a-1)

Step 1.

General Procedure E. To a solution of 1-6b (180 mg, 0.47 mmol) in DMF (6.0 mL) in a sealed tube was added 3-4 (224 mg, 0.57 mmol), tetrakis(triphenylphosphine)palladium (55 mg, 0.047 mmol), CuI (18 mg, 0.09 mmol) and triethylamine (0.2 mL, 1.5 mmol). The resulting solution was degassed and heated at 110° C. overnight. After cooled to rt, the mixture was partitioned between water and DCM. The aqueous layer was extracted with DCM. The combined organic phase was dried with anhydrous Na₂SO₄, filtered, and concentrated. The crude mixture was purified by flash column chromatography (EtOAc/ACN/NH₄OH=100/7/1 (v/v/v)) to afford 4a-1 (150 mg, 46% yield). LC-MS (ESI): m/z 692 [M+H]⁺.

### Methyl (S)-3-methyl-1-((S)-2-(5-(4-((2-((S)-4-methyl-2,5-dihydro-1H-pyrrol-2-yl)-1H-benzo[d]imidazol-6-yl)ethynyl)phenyl)-1H-imidazol-2-yl)pyrrolidin-1-yl)-1-oxobutan-2-ylcarbamate (4a-2)

Step 2. To a solution of 4a-1 (100 mg, 0.14 mmol) in DCM (2 mL) was added TFA (1.0 mL). The resulting solution was stirred at rt for 2 hrs. The solvent was removed. The residue was dried on vacuum for 1 hr. The crude 4a-2 was directly used in the next step without purification.

### Methyl N-[(2S)-1-[(2S)-2-{5-[4-(2-{2-[(2S)-1-[(2S)-2-[(methoxycarbonyl)amino]-3-methylbutanoyl]-4-methyl-2,5-dihydro-1H-pyrrol-2-yl]-1H-1,3-benzodiazol-6-yl}ethynyl)phenyl]-1H-imidazol-2-yl}pyrrolidin-1-yl]-3-methyl-1-oxobutan-2-yl]carbamate (4a-3)

Step 3. To a solution of N-Moc-L-Val-OH (30 mg, 0.17 mmol) in DMF (1 mL) was added HATU (82 mg, 0.21 mmol) and DIPEA (0.24 mL, 1.45 mmol). The resulting mixture was stirred at room temperature for 20 min, then poured into the solution of the crude 4a-2 (0.14 mmol) in DMF (1 mL). The solution was stirred at rt for another 2 hrs. The reaction mixture was partitioned between water and DCM. The aqueous layer was extracted with DCM. The combined organic phase was dried with anhydrous Na₂SO₄, filtered, and concentrated. The crude mixture was purified by prep HPLC to afford 4a-3 (23 mg). ¹H NMR (300 MHz, CD₃OD): δ 7.76 (m, 1H), 7.64 (m, 2H), 7.50-7.46 (m, 3H), 7.35 (m, 2H), 5.90 (s, 1H), 5.57 (s, 1H), 5.17 (t, 1H), 4.66 (dd, 2H), 4.19 (dd, 2H), 4.01 (m, 1H), 3.88 (m, 1H), 3.62 (s, 6H), 2.38-2.18 (m, 3H), 2.12-1.98 (m, 3H), 1.92 (s, 3H), 0.98-0.82 (m, 12H) ppm. LC-MS (ESI): m/z 749.5 [M+H]⁺, 375.4 [M+2H]²⁺.

### Methyl N-[(2S)-1-[(2S)-2-{5-[4-(2-{2-[(2S)-4-cyclopropyl-1-[(2S)-2-[(methoxycarbonyl)amino]-3-methylbutanoyl]-2,5-dihydro-1H-pyrrol-2-yl]-1H-1,3-benzodiazol-6-yl}ethynyl)phenyl]-1H-imidazol-2-yl}pyrrolidin-1-yl]-3-methyl-1-oxobutan-2-yl]carbamate (4a-5)

The title compound was prepared following procedures described for the synthesis of 4a-3 and substituting 1-6d for 1-6b in Step 1. LC-MS (ESI): m/z 775.4 [M+H]+.

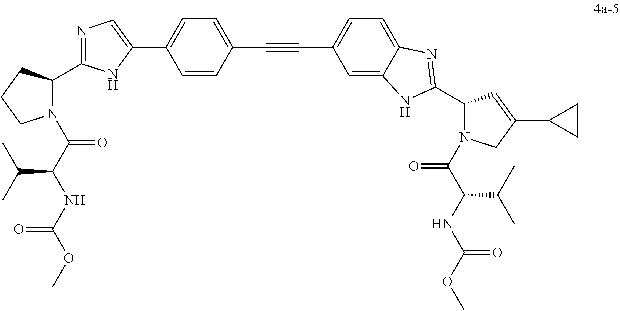 [SMILES: COC(=O)N[C@H](C(=O)N1CC(C2CC2)=C[C@H]1c1nc2ccc(C#Cc3ccc(-c4cnc([C@@H]5CCCN5C(=O)[C@@H](NC(=O)OC)C(C)C)[nH]4)cc3)cc2[nH]1)C(C)C]

### Methyl N-[(2S)-1-[(2S)-2-{6-[2-(4-{2-[(2S)-4-cyclopropyl-1-[(2S)-2-[(methoxycarbonyl)amino]-3-methylbutanoyl]-2,5-dihydro-1H-pyrrol-2-yl]-1H-imidazol-5-yl}phenyl)ethynyl]-1H-1,3-benzodiazol-2-yl}pyrrolidin-1-yl]-3-methyl-1-oxobutan-2-yl]carbamate (4a-6)

LC-MS (ESI): m/z 775.4 [M+H]⁺.

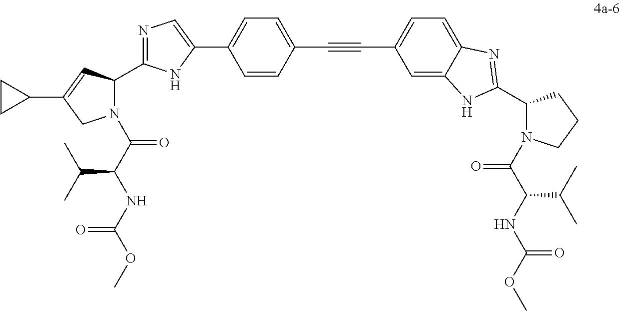 [SMILES: COC(=O)N[C@H](C(=O)N1CC(C2CC2)=C[C@H]1c1ncc(-c2ccc(C#Cc3ccc4nc([C@@H]5CCCN5C(=O)[C@@H](NC(=O)OC)C(C)C)[nH]c4c3)cc2)[nH]1)C(C)C]

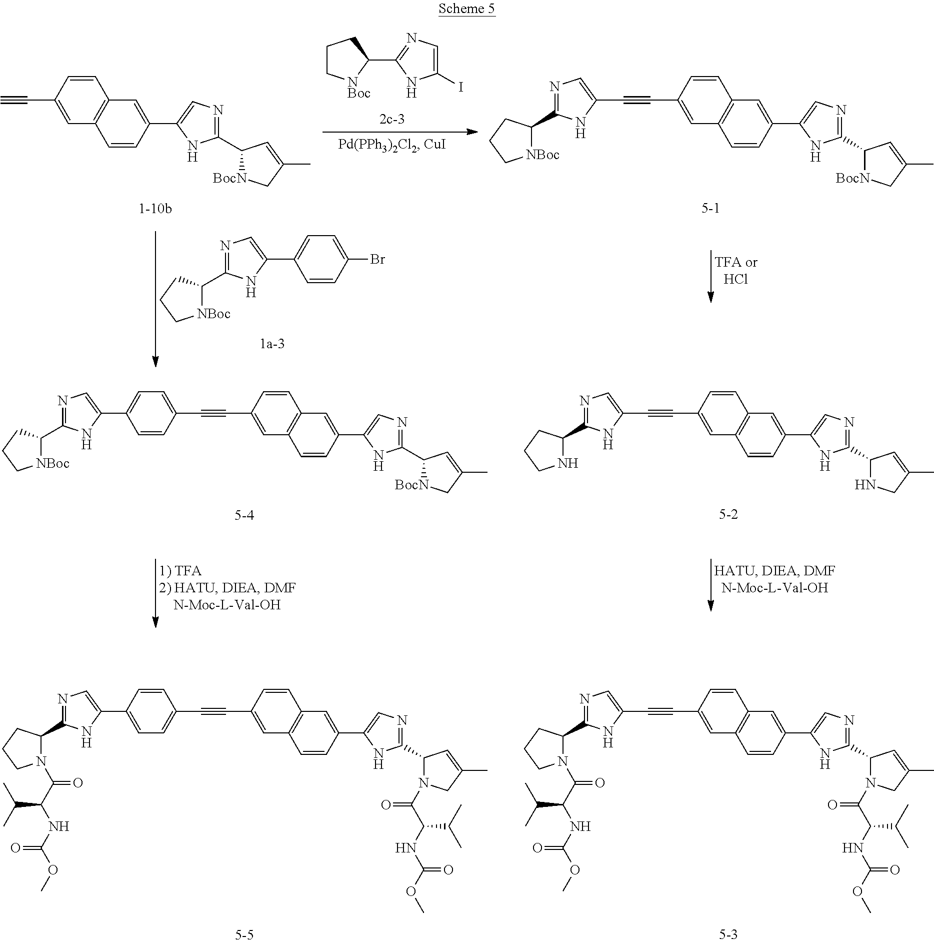

### (S)-tert-butyl 2-(5-(6-((2-((S)-1-(tert-butoxycarbonyl)pyrrolidin-2-yl)-1H-imidazol-5-yl)ethynyl)naphthalen-2-yl)-1H-imidazol-2-yl)-4-methyl-2,5-dihydro-1H-pyrrole-1-carboxylate (5-1)

Referring to Scheme 5. General Procedure E: Sonogashira coupling between an acetylene and an aryl bromide, chloride or triflate. To a solution of 1-10b (100 mg, 0.25 mmol) and 2c-3 (136 mg, 0.375 mmol) in triethylamine (2 mL) was added copper iodide (9.5 mg) and Pd(PPh₃)₂Cl₂ (18 mg) at rt. The reaction mixture after being thoroughly degassed was warmed up to 80° C. under the protection of nitrogen atmosphere and stirred overnight. After cooled to rt, the mixture was diluted with ethyl acetate (100 mL), washed with water and brine, and the organic phase was dried over anhydrous Na₂SO₄. After removing the solvents, the crude mixture was purified by flash column chromatography (Hex/EtOAc=1/2 (v/v)) to afford 5-1 (50 mg). LC-MS (ESI): m/z 636.4 [M+H]⁺.

### Methyl N-((2S)-1-((2S)-2-(5-(6-(2-(2-(1-((2S)-2-((methoxylcarbonyl)amino)-3-methylbutanoyl)pyrrolidin-2-yl)-1H-imidazole-5-yl)ethynyl)naphthalene-2-yl)-1H-imidazole-2-yl)-4-methyl)-2,5-dihydro-1H-pyrrol-1-yl)-3-methyl-1-oxobutan-2-yl)carbamate (5-3)

Deprotection of the Boc groups of 5-1 in the presence of a strong acid, such as TFA or HCl, followed amide formation with N-Moc-L-Val-OH gave 5-3. LC-MS (ESI): m/z 749.4 [M+H]⁺.

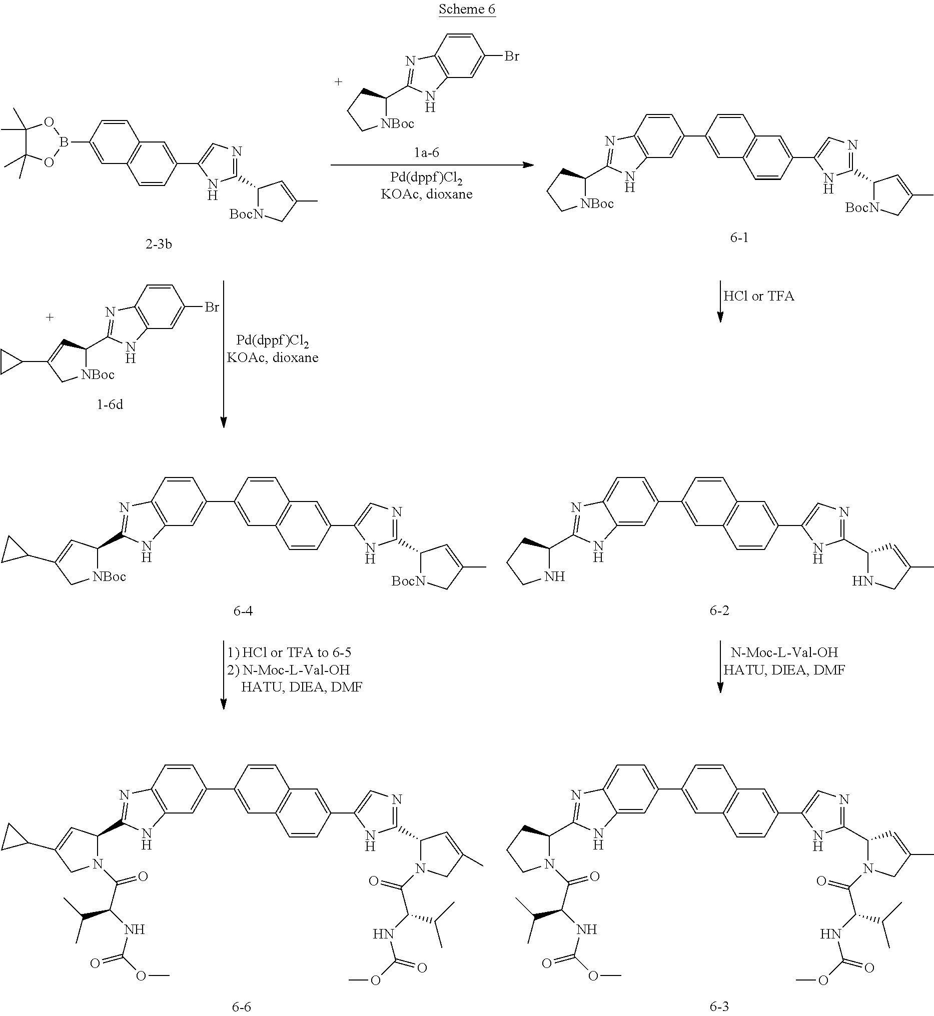

### (S)-tert-butyl 2-(5-(6-(2-((S)-1-(tert-butoxycarbonyl)pyrrolidin-2-yl)-1H-benzo[d]imidazol-6-yl)naphthalen-2-yl)-1H-imidazol-2-yl)-4-methyl-2,5-dihydro-1H-pyrrole-1-carboxylate (6-1)

Referring to Scheme 6. Following General Procedure F, 2-3b coupled with 1a-6 to give 6-1. LC-MS (ESI): m/z 661.3 [M+H]⁺.

### 6-(6-(2-((S)-4-methyl-2,5-dihydro-1H-pyrrol-2-yl)-1H-imidazol-5-yl)naphthalen-2-yl)-2-((S)-pyrrolidin-2-yl)-1H-benzo[d]imidazole (6-2)

Treatment of 6-1 with a strong acid, such as TFA or HCl gave 6-2. LC-MS (ESI): m/z 461.2 [M+H]⁺.

### Methyl N-((2S)-1-((2S)-2-(6-(6-(2-(2-(1-((2S)-2-((methoxylcarbonyl)amino)-3-methylbutanoyl)-4-methyl-2,5-dihydro-1H-pyrrol-2-yl)-1H-imidazole-5-yl)naphthalen-2-yl-1,3-benzodiazol-2-yl)pyrrolidin-1-yl)-3-methyl-1-oxobutan-2-yl)carbamate (6-3)

Amide formation of 6-2 with N-Moc-Val-OH (General Procedure G) gave 6-3. LC-MS (ESI): m/z 775.4 [M+H]⁺.

### (S)-tert-butyl 2-(5-(6-(2-((S)-1-(tert-butoxycarbonyl)-4-cyclopropyl-2,5-dihydro-1H-pyrrol-2-yl)-1H-benzo[d]imidazol-6-yl)naphthalen-2-yl)-1H-imidazol-2-yl)-4-methyl-2,5-dihydro-1H-pyrrole-1-carboxylate (6-4)

Following General Procedure F, 2-3b coupled with 1-6d to give 6-4. LC-MS (ESI): m/z 699.4 [M+H]⁺.

### 2-((S)-4-cyclopropyl-2,5-dihydro-1H-pyrrol-2-yl)-6-(6-(2-((S)-4-methyl-2,5-dihydro-1H-pyrrol-2-yl)-1H-imidazol-5-yl)naphthalen-2-yl)-1H-benzo[d]imidazole (6-5)

Treatment of 6-1 with a strong acid, such as TFA or HCl gave 6-5. LC-MS (ESI): m/z 499.3 [M+H]+.

### Methyl N-((2S)-1-((2S)-4-cyclopropyl-2-(6-(6-(2-((2S)-1-((2S)-2-((methoxylcarbonyl)amino)-3-methylbutanoyl)-4-methyl-2,5-dihydro-1H-pyrrol-2-yl)-1H-imidazole-5-yl)naphthalen-2-yl-1,3-benzodiazol-2-yl)-2,5-dihydro-1H-pyrrol-1-yl)-3-methyl-1-oxobutan-2-yl)carbamate (6-6)

Amide formation of 6-5 with N-Moc-Val-OH (General Procedure G) gave 6-6. LC-MS (ESI): m/z 813.4 [M+H]⁺.

### Methyl N4(2S)-1-((2S)-2-(5-(6-(2-((2S)-1-((2S)-2-((methoxycarbonyl)amino]-3-methylbutanoyl)-4-methyl-2,5-dihydro-1H-pyrrol-2-yl)-1H-1,3-benzodiazol-6-yl)naphthalen-2-yl)-1H-imidazol-2-yl)pyrrolidin-1-yl)-3-methyl-1-oxobutan-2-yl]carbamate (6-7)

Following the procedure described in Scheme 6, 6-7 was obtained. LC-MS (ESI): m/z 775.4 [M+H]⁺.

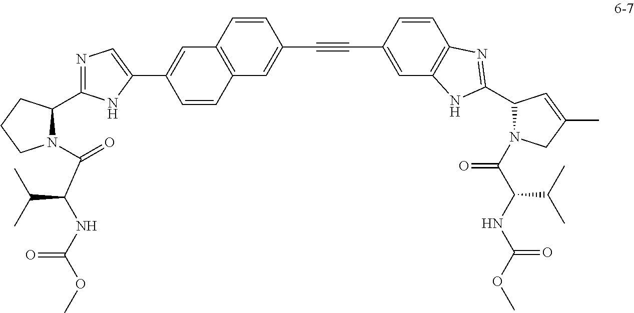

### Methyl N-((2S)-1-((2S)-2-(5-(6-(2-((2S)-1-((2S)-2-((methoxycarbonyl)amino]-3-methylbutanoyl)-4-cyclopropyl-2,5-dihydro-1H-pyrrol-2-yl)-1H-1,3-benzodiazol-6-yl)naphthalen-2-yl)-1H-imidazol-2-yl)pyrrolidin-1-yl)-3-methyl-1-oxobutan-2-yl]carbamate (6-8)

Following the procedure described in Scheme 6, compound 6-8 was obtained. LC-MS (ESI): m/z 801.4 [M+H]⁺.

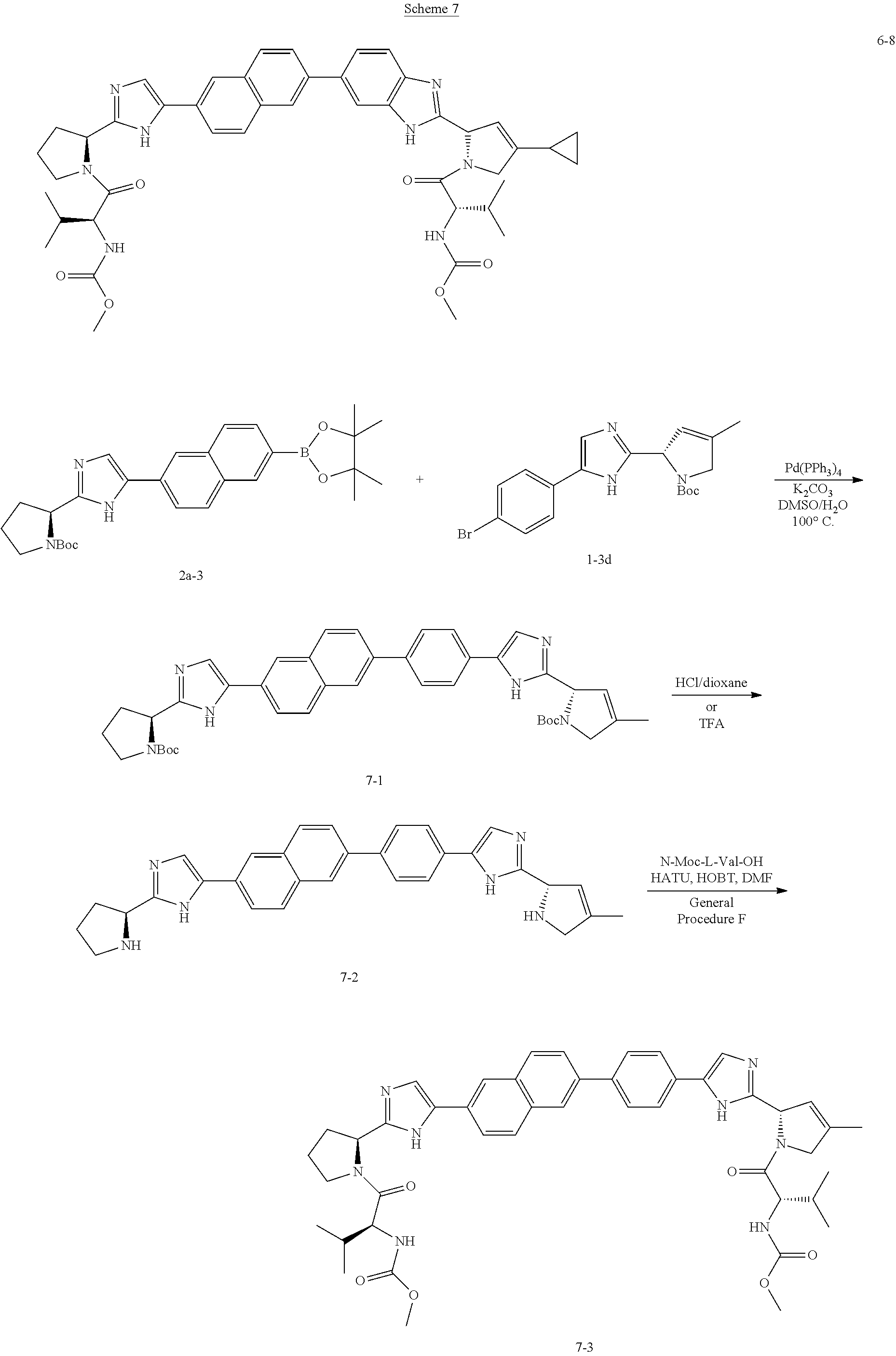

### (S)-tert-butyl 2-(5-(4-(6-(2-((S)-1-(tert-butoxycarbonyl)pyrrolidin-2-yl)-1H-imidazol-5-yl)naphthalen-2-yl)phenyl)-1H-imidazol-2-yl)-4-methyl-2,5-dihydro-1H-pyrrole-1-carboxylate (7-1)

Referring to Scheme 7. General Procedure F: To a solution of 2-1b (200 mg, 0.50 mmol) in DMSO (5.0 mL) and H₂O (1.5 mL) was added 1-3b (245 mg, 0.5 mmol), tetrakis(triphenylphosphine)palladium (58 mg, 0.05 mmol) and potassium carbonate (207 mg, 1.5 mmol). The resulting solution was degassed and heated at 100° C. for 2 hrs. The reaction mixture was cooled to room temperature and poured into water (100 mL). The precipitate was collected through filtration. The crude mixture was purified by flash column chromatography (EtOAc/Acetonitrile/NH₄OH=100/7/1 (v/v/v)) to afford the compound 7-1 (240 mg, 70% yield). LC-MS (ESI): m/z 688 [M+H]⁺.

### 2-((S)-4-methyl-2,5-dihydro-1H-pyrrol-2-yl)-5-(4-(6-(2-((S)-pyrrolidin-2-yl)-1H-imidazol-5-yl)naphthalen-2-yl)phenyl)-1H-imidazole (7-2)

To a solution of 7-1 (100 mg, 0.146 mmol) in DCM (2 mL) was added TFA (1 mL). The resulting solution was stirred at rt for 2 hrs. The solvent was removed. The residue was dried on vacuum for 1 hr. The deprotected product 7-2 was directly used in the next step. LC-MS (ESI): m/z 487 [M+H]⁺.

### Methyl N-[(2S)-1-[(2S)-2-{5-[6-(4-{2-[(2S)-1-[(2S)-2-[(methoxycarbonyl)amino]-3-methylbutanoyl]-4-methyl-2,5-dihydro-1H-pyrrol-2-yl]-1H-imidazol-5-yl}phenyl)naphthalen-2-yl]-1H-imidazol-2-yl}pyrrolidin-1-yl]-3-methyl-1-oxobutan-2-yl]carbamate (7-3)

General Procedure G.

To a solution of N-Moc-L-Val-OH (51.2 mg, 0.29 mmol) in DMF (1.5 mL) was added HATU (167 mg, 0.44 mmol) and DIPEA (0.29 mL, 1.75 mmol). The resulting mixture was stirred at rt for 20 min, the mixture was then transferred to a solution of the crude 7-2 (0.146 mmol) in DMF (1.5 mL). The entire mixture was stirred at rt for another 2 hrs. The reaction mixture was partitioned between water and DCM. The aqueous layer was extracted with DCM. The combined organic phase was dried with anhydrous Na₂SO₄, filtered, and concentrated. The crude mixture was purified by prep HPLC to afford 7-3 (30 mg). ¹H NMR (300 MHz, CD₃OD): δ 8.30 (s, 1H), 8.26 (s, 1H), 8.12 (d, 1H), 8.08 (d, 1H), 7.99-7.84 (m, 8H), 6.04 (bs, 1H), 5.65 (s, 1H), 5.28 (t, 1H), 4.80-4.59 (dd, 2H), 4.25 (d, 1H), 4.15-4.06 (m, 2H), 3.92 (m, 1H), 3.66 (s, 6H), 2.60 (m, 1H), 2.35-2.18 (m, 3H), 2.14-2.04 (m, 2H), 2.03 (s, 3H), 0.98-0.82 (m, 12H) ppm. LC-MS (ESI): m/z 801.6 [M+H]⁺, 799.5 M−H]⁻.

### Methyl N-[(2S)-1-[(2S)-2-{5-[6-(4-{2-[(2S)-4-cyclopropyl-1-[(2S)-2-[(methoxycarbonyl)amino]-3-methylbutanoyl]-2,5-dihydro-1H-pyrrol-2-yl]-1H-imidazol-5-yl}phenyl)naphthalen-2-yl]-1H-imidazol-2-yl}pyrrolidin-1-yl]-3-methyl-1-oxobutan-2-yl]carbamate (7-4)

Following the same procedure as described for thr synthesis of 7-3 by replacing 1-3b with 1-3d, 7-4 was obtained. LC-MS (ESI): m/z 827.4 [M+H]⁺.

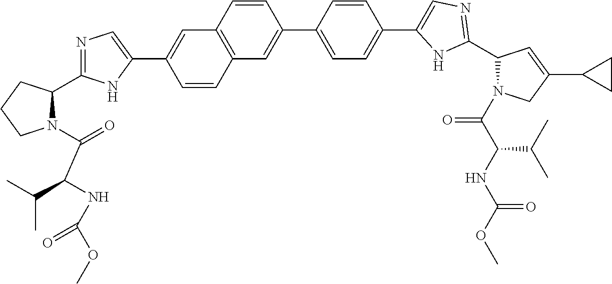

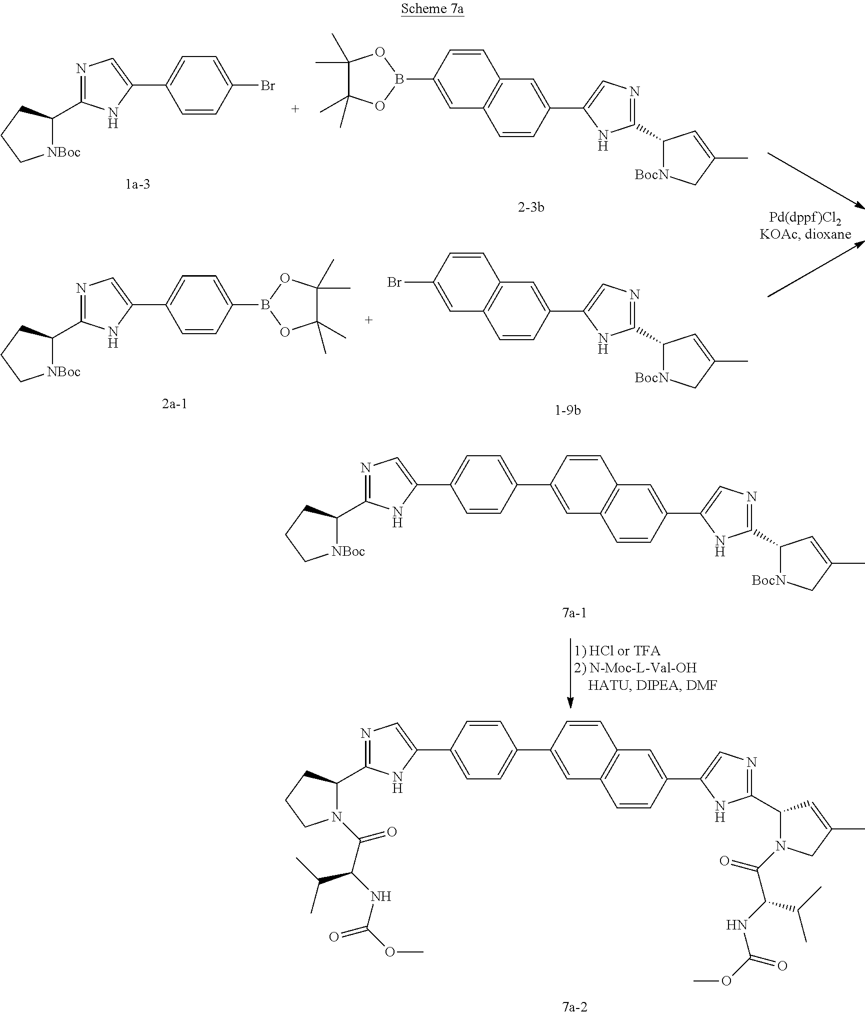

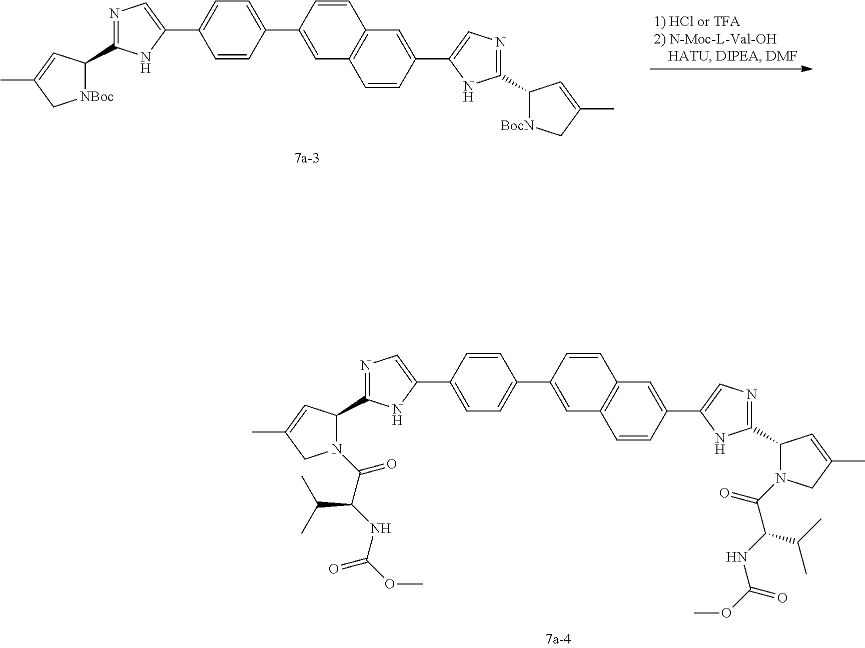

### (S)-tert-butyl 2-(5-(6-(4-(2-((S)-1-(tert-butoxycarbonyl)pyrrolidin-2-yl)-1H-imidazol-5-yl)phenyl)naphthalen-2-yl)-1H-imidazol-2-yl)-4-methyl-2,5-dihydro-1H-pyrrole-1-carboxylate (7a-1)

Referring to Scheme 7a. Following General Procedure F, either 1a-3 coupled with 2-3b or 2a-1 coupled with 1-9b gave 7a-1. LC-MS (ESI): m/z 687.4 [M+H]⁺.

### Methyl N-((2S)-1-((2S)-2-(5-(4-(6-(2-((2S)-1-((2S)-((methoxylcarbonyl)amino)-3-methylbutanoyl)-4-methyl-2,5-dihydron-1H-imidazole-5-yl)naphthalene-2-yl)phenyl)-1H-imidazole-2-yl)-pyrrolidin-1-yl)-3-methyl-1-oxobutan-2-yl)carbamate (7a-2)

Treatment of compound 7a-1 with either TFA or 4 N HCl in dioxane, followed by Procedure G gave compound 7a-2. LC-MS (ESI): m/z 801.4 [M+H]⁺.

### (S)-tert-butyl 2-(5-(4-(6-(2-((S)-1-(tert-butoxycarbonyl)-4-methyl-2,5-dihydro-1H-pyrrol-2-yl)-1H-imidazol-5-yl)naphthalen-2-yl)phenyl)-1H-imidazol-2-yl)-4-methyl-2,5-dihydro-1H-pyrrole-1-carboxylate (7a-3)

Following General Procedure F, either compound 1-3b coupled with compound 2-3b or compound 1-9b coupled with compound 2-1b gave compound 7a-3. LC-MS (ESI): m/z 699.4 [M+H]⁺.

### Methyl N-((2S)-1-((2S)-2-(5-(4-(6-(2-((2S)-1-((2S)-2-((methoxycarbonyl)amino)-3-methylbutanoyl)-4-methyl-2,5-dihydro-1H-pyrrol-2-yl)-1H-imidazole-5-yl)naphthalene-2-yl)phenyl)-1H-imidazol-2-yl)-4-methyl-2,5-dihydro-1H-pyrrol-1-yl)-3-methyl-1-oxobutan-2-yl)carbamate (7a-4)

Treatment of compound 7a-3 with either TFA or 4 N HCl in dioxane, followed by Procedure G gave compound 7a-4. LC-MS (ESI): m/z 813.4 [M+H]⁺,

### Methyl N-((2S)-1-((2S)-2-(5-(4-(6-(2 ((2S)-1-((2S)-2-((methoxycarbonyl)amino)-3-methylbutanoyl)-4-methyl-2,5-dihydro-1H-pyrrol-2-yl)-1H-imidazole-5-yl)naphthalene-2-yl)phenyl)-1H-imidazol-2-yl)-4-cyclopropyl-2,5-dihydro-1H-pyrrol-1-yl)-3-methyl-1-oxobutan-2-yl)carbamate (7a-5).

Following the procedure described in Scheme 7a by replacing compound 1a-3 with 1-3d or compound 2a-1 with 2-1d, compound 7a-5 was obtained. LC-MS (ESI): m/z 839.4 [M+H]⁺.

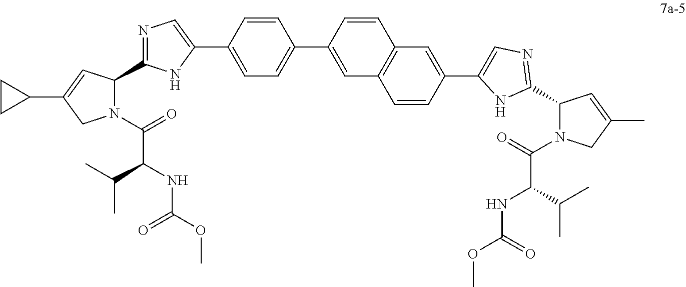 [SMILES: COC(=O)N[C@H](C(=O)N1CC(C)=C[C@H]1c1ncc(-c2ccc3cc(-c4ccc(-c5cnc([C@@H]6C=C(C7CC7)CN6C(=O)[C@@H](NC(=O)OC)C(C)C)[nH]5)cc4)ccc3c2)[nH]1)C(C)C]

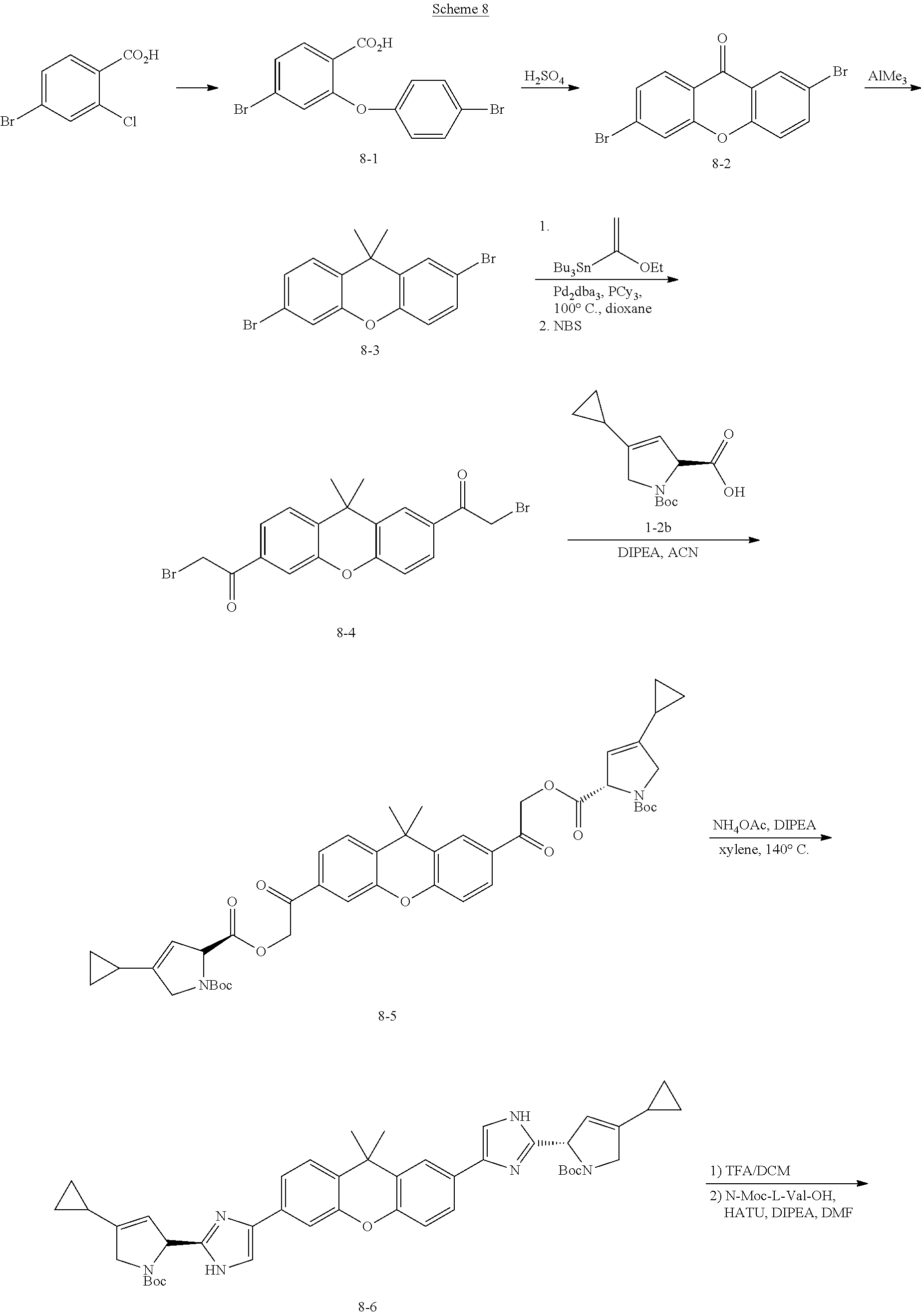

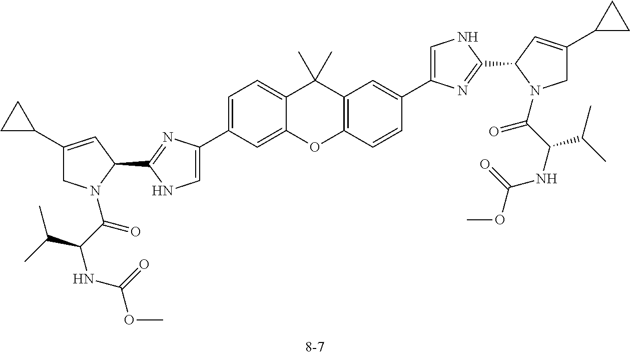 [SMILES: COC(=O)N[C@H](C(=O)N1CC(C2CC2)=C[C@H]1c1nc(-c2ccc3c(c2)Oc2ccc(-c4c[nH]c([C@@H]5C=C(C6CC6)CN5C(=O)[C@@H](NC(=O)OC)C(C)C)n4)cc2C3(C)C)c[nH]1)C(C)C]

Referring to Scheme 8, Step 1. To a solution of 4-bromo-2-chlorobenzoic acid (18.4 g, 83.9 mmol) and 4-bromophenol (24 g, 109 mmol) in nitrobenzene was added cesium carbonate (82 g, 251.7 mmol). The resulting solution was heated at 170° C. with a condenser for 1 day. The reaction mixture was cooled to 70° C. and filtered at this temperature. The residue was washed with toluene. The organic layer was removed by vacuum distillation till a thick dark residue remained. To the dark residue was added aqueous HCl (1 N, 400 mL) and DCM (200 mL). The resulting solution was stirred until dark oil dispersed into DCM solution. The mixture was filtered. The organic layer was dried over anhydrous Na₂SO₄ and concentrated to afford the crude product. The residue was purified by column chromatography on silica gel, eluted first with DCM and then with a mixture of DCM and MeOH to give 8-1.

Step 2.

Compound 8-1 (16 g, 5:3 ratio, 44.3 mmol) was treated with concentrated sulfuric acid (95 mL). The solution was heated at 105° C. for 2 hrs. The reaction mixture was cooled and poured into ice water. The product precipitated out and was collected by filtration, washed with Et₂O and H₂O. The solid was dried and further purified by flash column chromatography on silica gel (eluents: Hex/EtOAc=9/1 (v/v) to pure EtOAc and to pure DCM) to afford 8-2 (12 g).

Step 3.

Trimethylaluminum (2.4 mL, 2 M in hexanes, 4.80 mmol) was added dropwise to a degassed stirred solution of 2,6-dibromo-9H-xanthen-9-one (8-2) (500 mg, 1.412 mmol) in toluene (8 mL) at 0° C. The resulting solution was allowed to warm up to rt and left to stir for 16 hrs. The crude reaction mixture was poured into ice-cold 1 N HCl aq. (200 mL), and the aqueous layer was washed with DCM (2×150 mL), dried over anhydrous MgSO₄, filtered and solvents were removed in vacuo to give 2,6-dibromo-9,9-dimethyl-9H-xanthene (8-3) (482 mg, 93% yield) as a white solid. ¹H NMR (CDCl₃): δ 7.77-7.74 (1H, m), 7.55-7.51 (1H, m), 7.44-7.40 (1H, m), 7.33-7.29 (2H, m), 7.06-7.02 (1H, m), 1.58 (6H, s) ppm.

Step 4.

A seal tube was charged with Pd₂(dba)₃ (55 mg, 0.06 mmol), tricyclohexylphosphine (34 mg, 0.12 mmol) and dioxane (20 mL). The resulting solution was bubbled with N₂ for 5 mins and stirred for 30 mins at rt. Compound 8-3 (1.0 g, 2.71 mmol), tri-n-butyl(1-ethoxyvinyl)stannane (2.1 mL, 6.20 mmol) and cesium fluoride (1.8 g, 11.9 mmol) were added while being protected under an atmosphere of N₂. After stirring 30 hrs at 145° C., the reaction mixture was cooled to rt and filtered through a pad of Celite° 545, which was rinsed with dioxane (20 mL).

The combined dioxane solution from above was diluted with H₂O (10 mL) and cooled to 0° C. NBS (1.00 g, 5.62 mmol) was then added in portions over 15 mins. After about 30 mins stirring, the volatile component was removed in vacuo, and the residue was partitioned between DCM (100 mL) and water. The aqueous layer was extracted with DCM (3×20 mL). The combined organic phases were washed by brine, water, dried over Na₂SO₄. After concentration to remove all solvents, the crude residue was triturated by DCM (3×15 mL) to remove most of stannane derivative to afford 1,1′-(9,9-dimethyl-9H-xanthene-2,6-diyl)bis(2-bromoethanone) (8-4) (1.1 g). ¹H NMR (300 MHz, CDCl₃): δ 8.14 (d, J=2.2 Hz, 1H), 7.86 (dd, J₁=8.5 Hz, J₂=2.2 Hz, 1H), 7.75 (dd, J₁=8.1 Hz, J₂=1.7 Hz, 1H), 7.70 (d, J=1.7 Hz), 7.56 (d, J=8.0 Hz), 7.16 (d, J=8.5 Hz), 4.44 (s, 2H), 4.42 (s, 2H), 1.70 (s, 6H) ppm.

Step 5.

General Procedure H (Steps 5 and 6). To a suspension of 8-4 (180 mg, 0.40 mmol) in CH₃CN (6 mL) was added 1-2b (210 mg, 0.83 mmol) and N,N-Diisopropylethylamine (0.144 mL, 0.826 mmol). The mixture was stirred overnight. The volatile component was removed in vacuo, and the residue was partitioned between water and DCM. The aqueous layer was extracted with DCM. The combined organic phases were washed by brine, saturated sodium carbonate, and water, and dried over anhydrous Na₂SO₄. After concentration, the crude mixture was purified by flash column chromatography (hexanes/ethyl acetate=2/1 (v/v)) to afford compound 8-5 (230 mg). LC-MS (ESI): m/z 819.5 [M+Na]⁺.

Step 6.

A mixture of 8-5 (230 mg, 0.289 mmol), ammonium acetate (445 mg, 5.78 mmol) and N,N-Diisopropylethylamine (1.00 mL, 5.78 mmol) in xylene (4 mL) in a sealed tube was stirred at 140° C. for 2 hrs. LC-MS showed the reaction was completed. The solvent was removed in vacuo, and the residue was partitioned between water and DCM. The aqueous layer was extracted with DCM. The combined organic phase was washed by brine, water, and dried over anhydrous Na₂SO₄. After removing the solvents, the crude mixture was purified by flash column chromatography (Hex/EtOAc=1/2 (v/v)) to afford compound 8-6 (120 mg). LC-MS (ESI): m/z 757.4 [M+H]⁺.

Step 7.

To a stirred solution of 8-6 (60 mg) in dichloromethane (5 mL) was added trifluoroacetic acid (1 mL). After 3 hrs, the reaction was concentrated to dryness. The de-Boced intermediate was dissolved in DMF (1 mL). To the solution were added DIPEA (0.139 mL), N-Moc-L-Val-OH (28 mg) and HATU (61 mg) subsequently. After 1 hr stirring, the reaction was diluted with water. The reaction was extracted by dichloromethane. The combined organic solution was washed with brine and water, dried over anhydrous Na₂SO₄, filtered, and concentrated. The resulting crude product was purified by prep-HPLC (Phenomenex, C18-Luna column, H₂O-ACN, 0.1% HCO₂H) to give compound 8-7 (17 mg). ¹H NMR (300 MHz, CDCl₃): δ 8.29 (s, 2H), 7.47 (br s, 1H), 7.19-7.14 (m, 4H), 7.08-7.05 (m, 1H), 6.98-6.94 (m, 1H), 6.79-6.72 (m, 1H), 5.92 (br s, 2H), 5.56 (br s, 2H), 4.53 (br s, 4H), 4.29 (t, J=8.0 Hz, 2H), 3.68 (br s, 6H), 2.00 (s, 6H), 1.60-1.40 (m, 6H), 0.95-0.80 (m, 14H), 0.80-0.60 (m, 4H) ppm; LC-MS (ESI): m/z 871.4 [M+H]⁺.

### Methyl N-[(2S)-1-[(2S)-2-[5-(7-{2-[(2S)-1-[(2S)-2-[(methoxycarbonyl)amino]-3-methylbutanoyl]-4-methyl-2,5-dihydro-1H-pyrrol-2-yl]-1H-imidazol-5-yl}-9,9-dimethyl-9H-xanthen-3-yl)-1H-imidazol-2-yl]-4-methyl-2,5-dihydro-1H-pyrrol-1-yl]-3-methyl-1-oxobutan-2-yl]carbamate (8-8)

Following the procedure described in Scheme 8 by replacing 1-2d with 1-2b, 8-8 was obtained. LC-MS (ESI): m/z 819.4 [M+H]⁺.

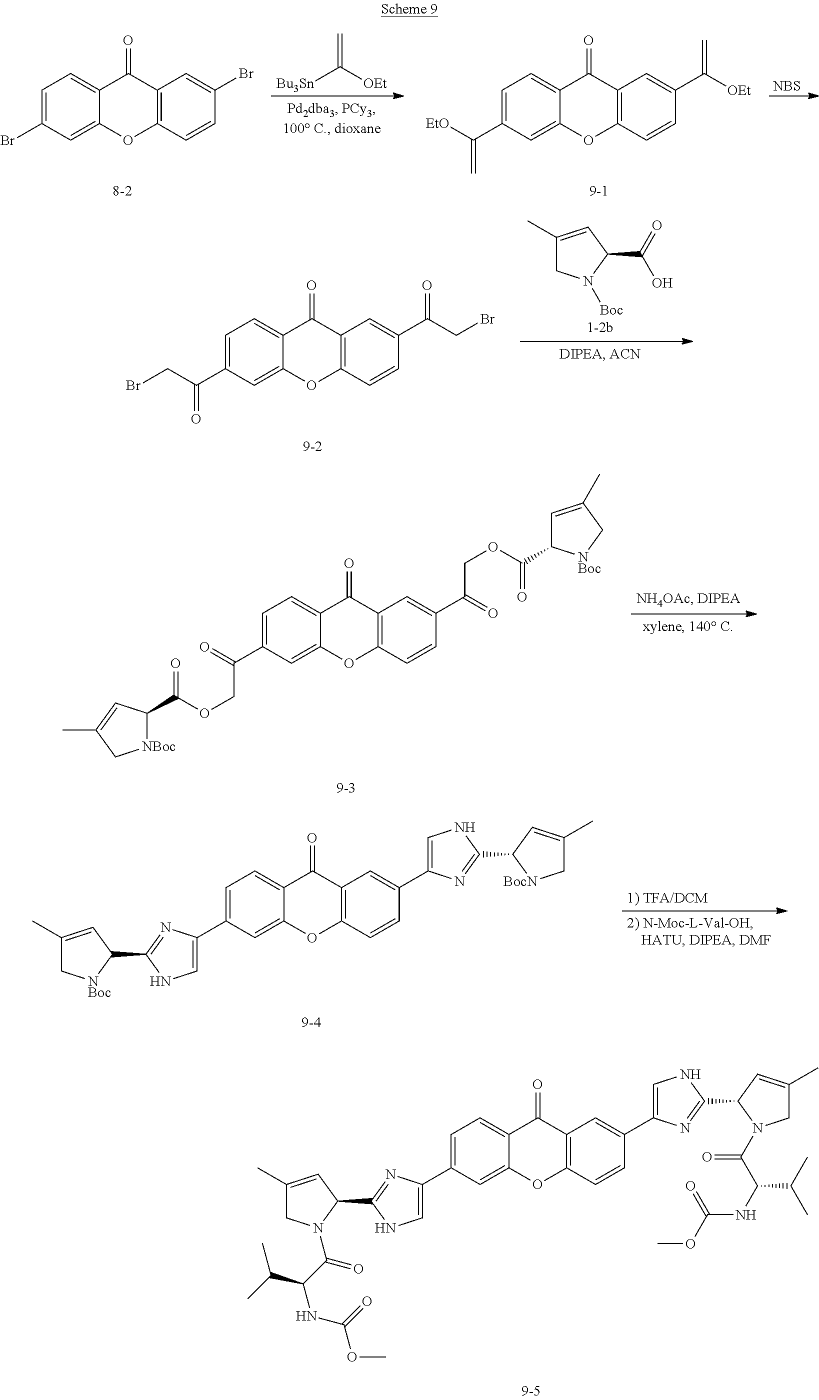

### Methyl N-((2S)-1-((2S)-2-(5-(7-(2-((2S)-1-((2S)-2-((methoxycarbonyl)amino)-3-methylbutanoyl)-4-methyl-2,5-dihydro-1H-pyrrol-2-yl)-1H-imidazol-5-yl)-9-oxo-9H-xanthen-3-yl)-1H-imidazol-2-yl)-4-methyl-2,5-dihydro-1H-pyrrol-1-yl)-3-methyl-1-oxobutan-2-yl)carbamate (9-5)

Following the procedure described in Scheme 8 by replacing both 8-3 with 8-2 and 1-2d with 1-2b, 9-5 was obtained. LC-MS (ESI): m/z 805.4 [M+H]⁺.

### Methyl N-((2S)-1-((2S)-4-cyclopropyl-2-(5-(7-(2-((2S)-4-cyclopropyl-1-((2S)-2-((methoxycarbonyl)amino)-3-methylbutanoyl)-2,5-dihydro-1H-pyrrol-2-yl)-1H-imidazol-5-yl)-9-oxo-9H-xanthen-3-yl)-1H-imidazol-2-yl)-2,5-dihydro-1H-pyrrol-1-yl)-3-methyl-1-oxobutan-2-yl)carbamate (9-6)

Following the procedure described in Scheme 8 by replacing 8-3 with 8-2, 9-6 was obtained. LC-MS (ESI): m/z 857.4 [M+H]⁺.

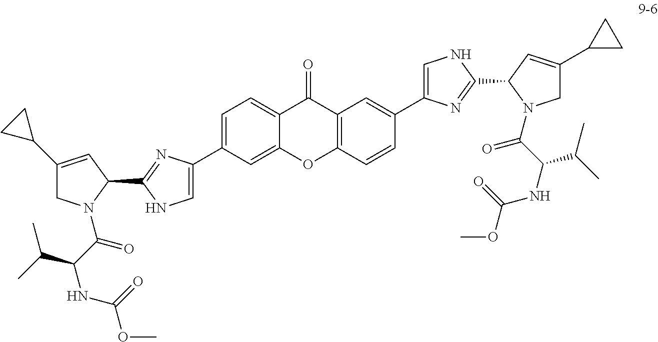 [SMILES: COC(=O)N[C@H](C(=O)N1CC(C2CC2)=C[C@H]1c1nc(-c2ccc3c(=O)c4cc(-c5c[nH]c([C@@H]6C=C(C7CC7)CN6C(=O)[C@@H](NC(=O)OC)C(C)C)n5)ccc4oc3c2)c[nH]1)C(C)C]

Additional Synthetic Schemes

Provided below are additional synthetic schemes useful in making the disclosed compounds. All compound numbering is restarted in this section, “Additional Synthetic Schemes.”

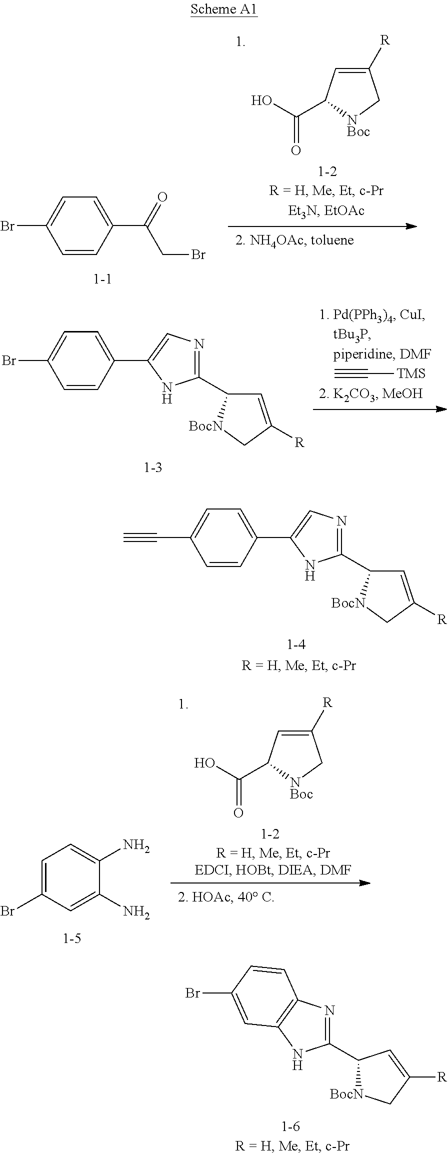

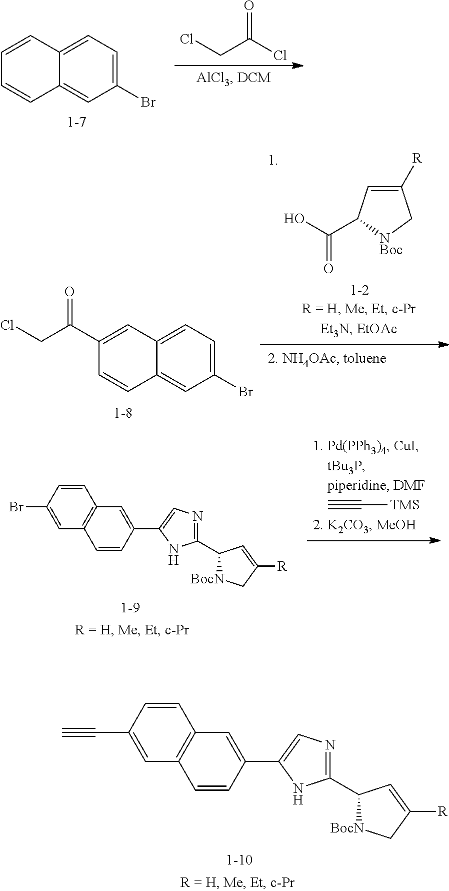

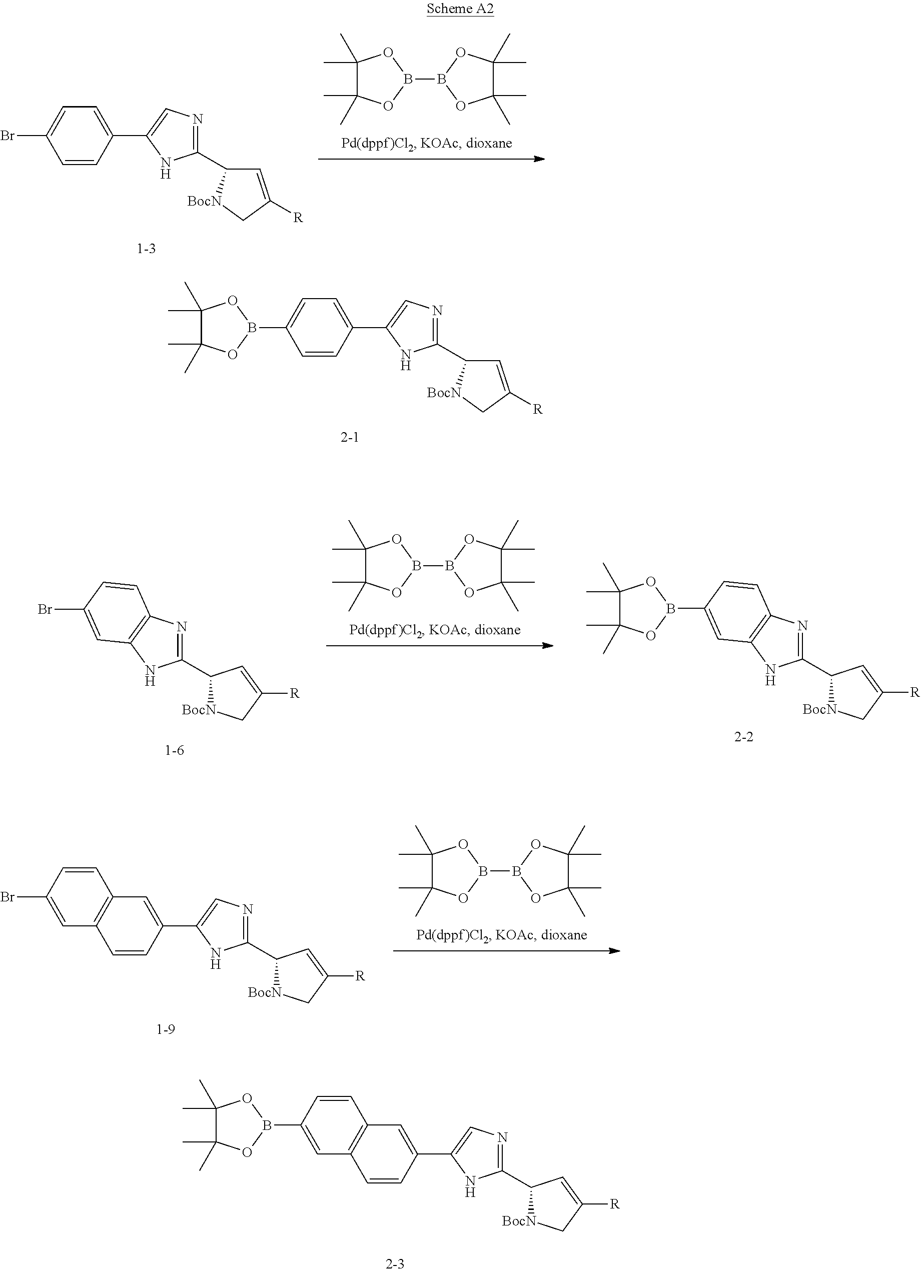

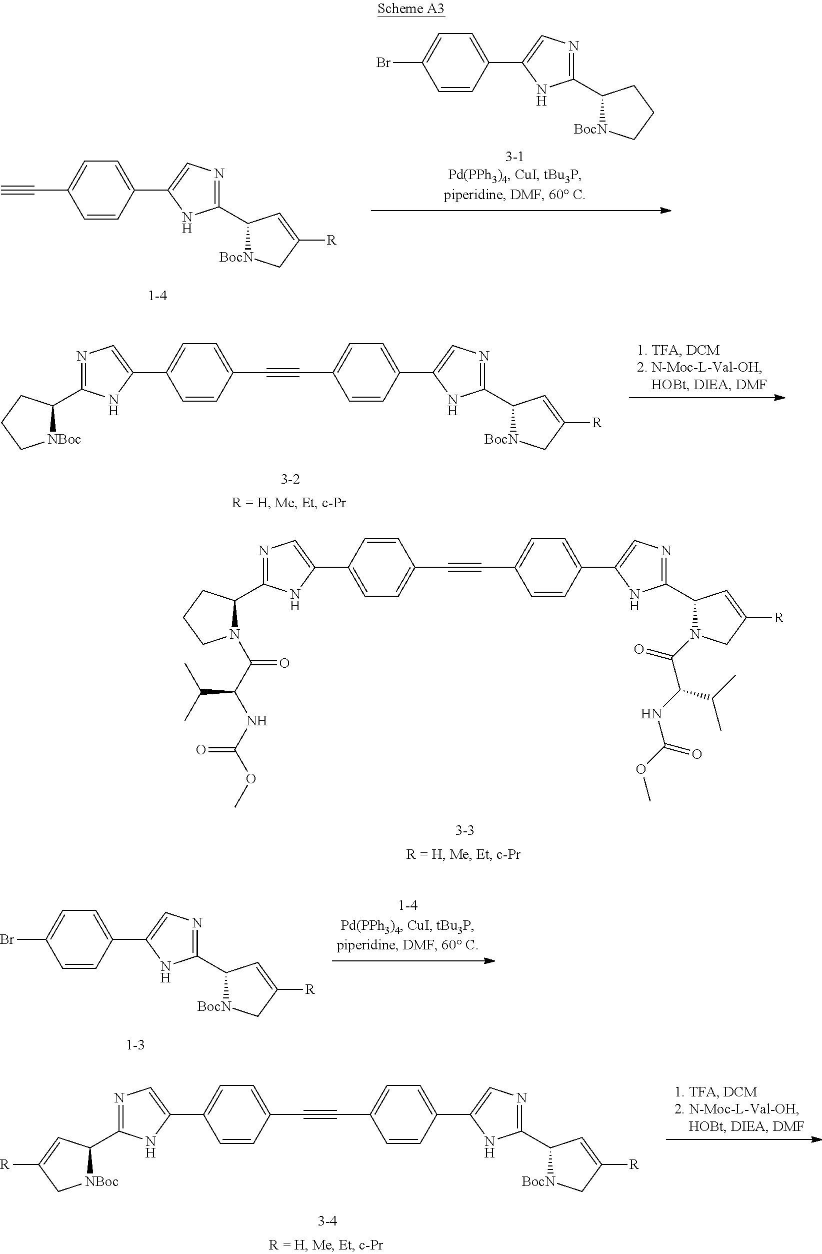

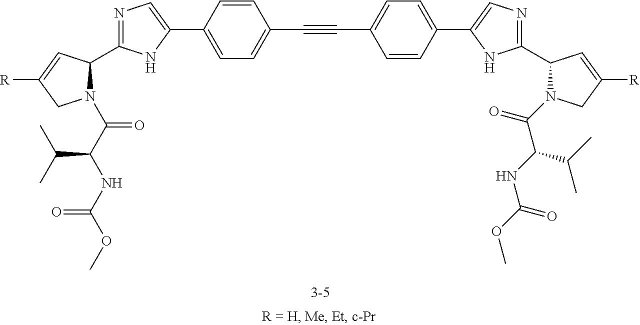

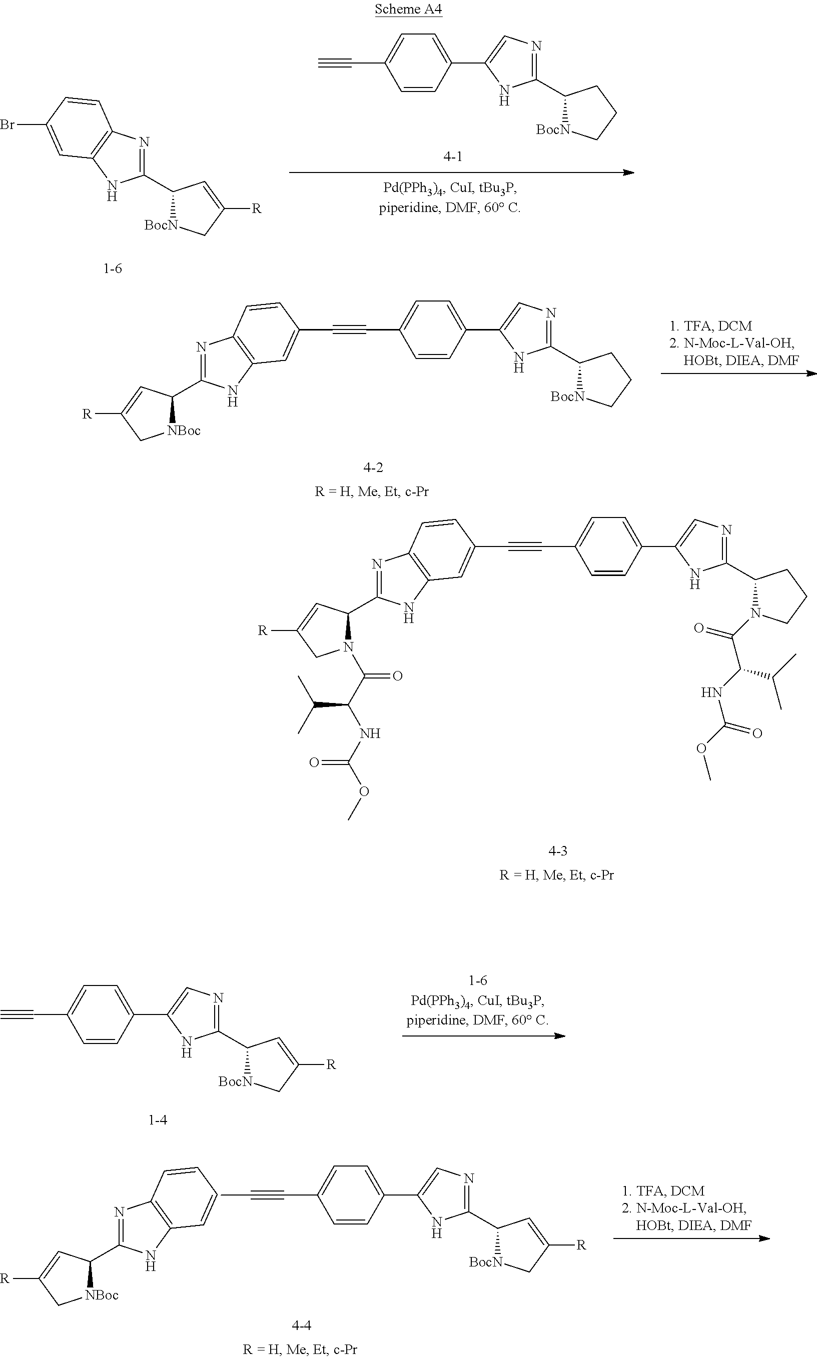

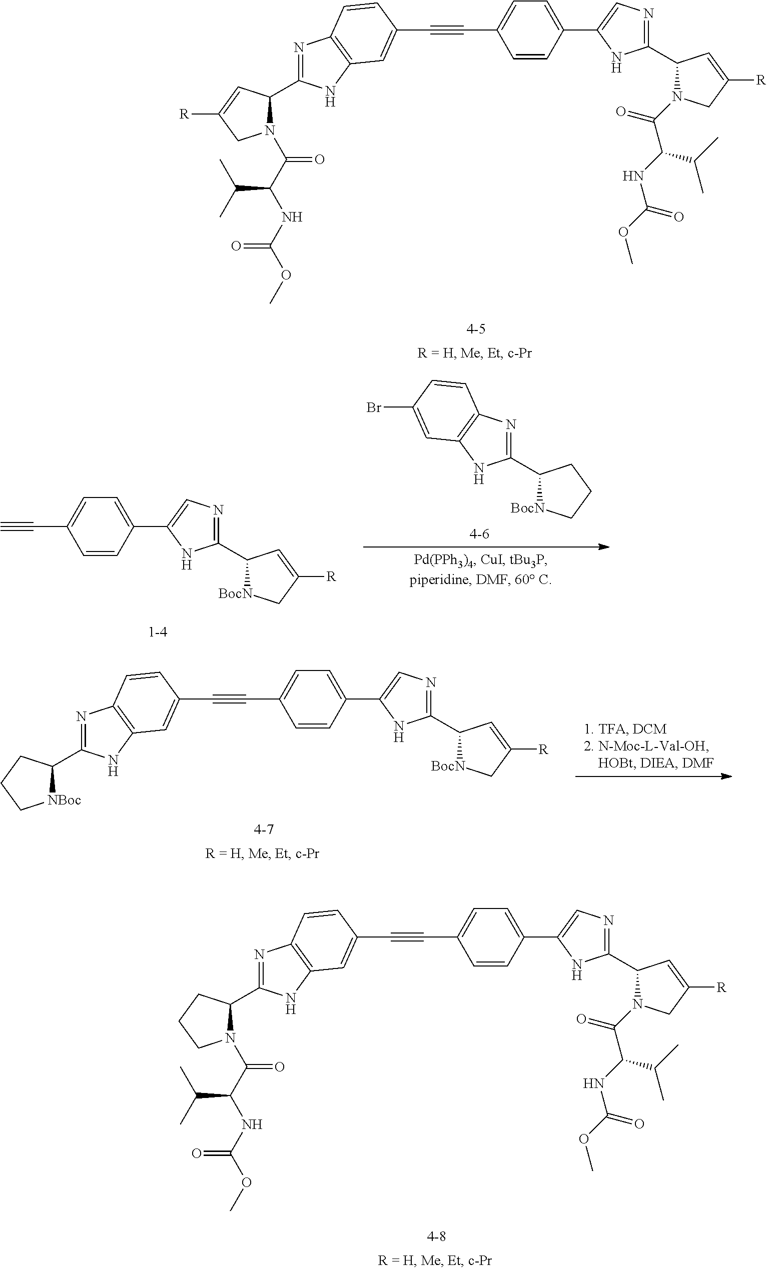

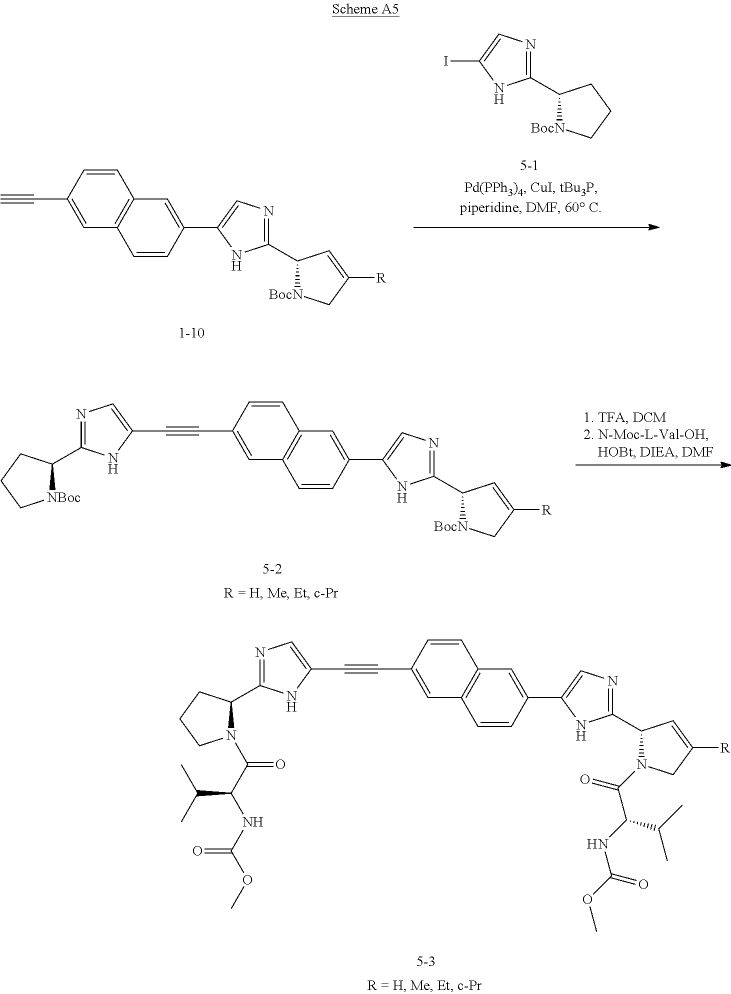

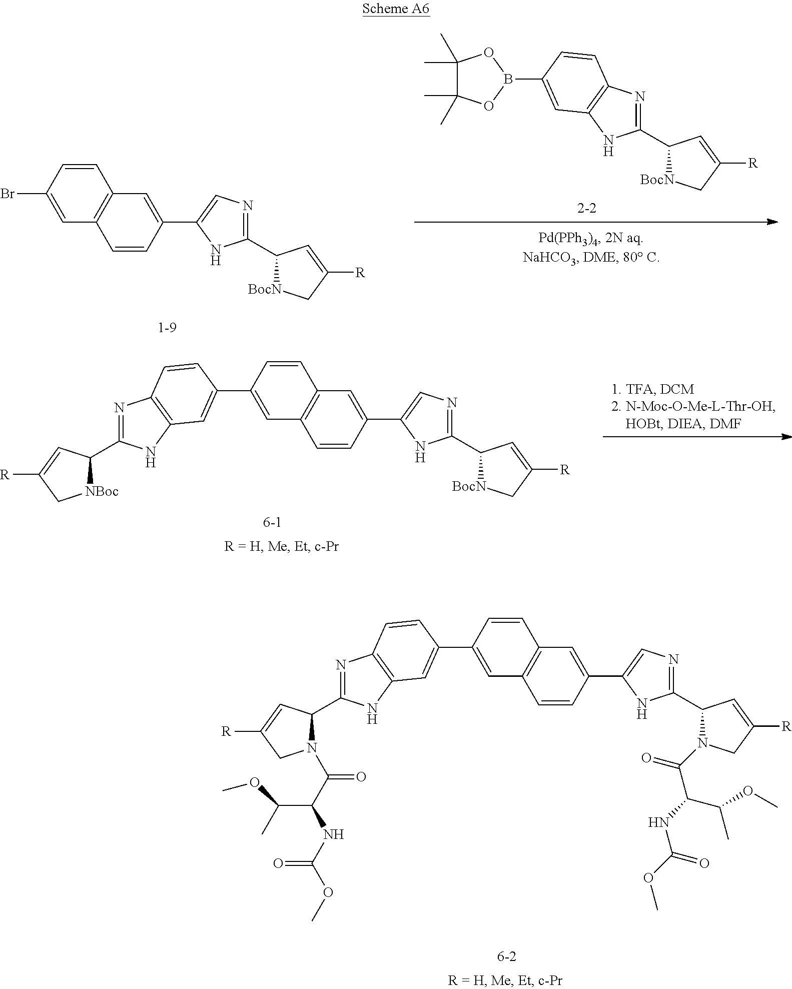

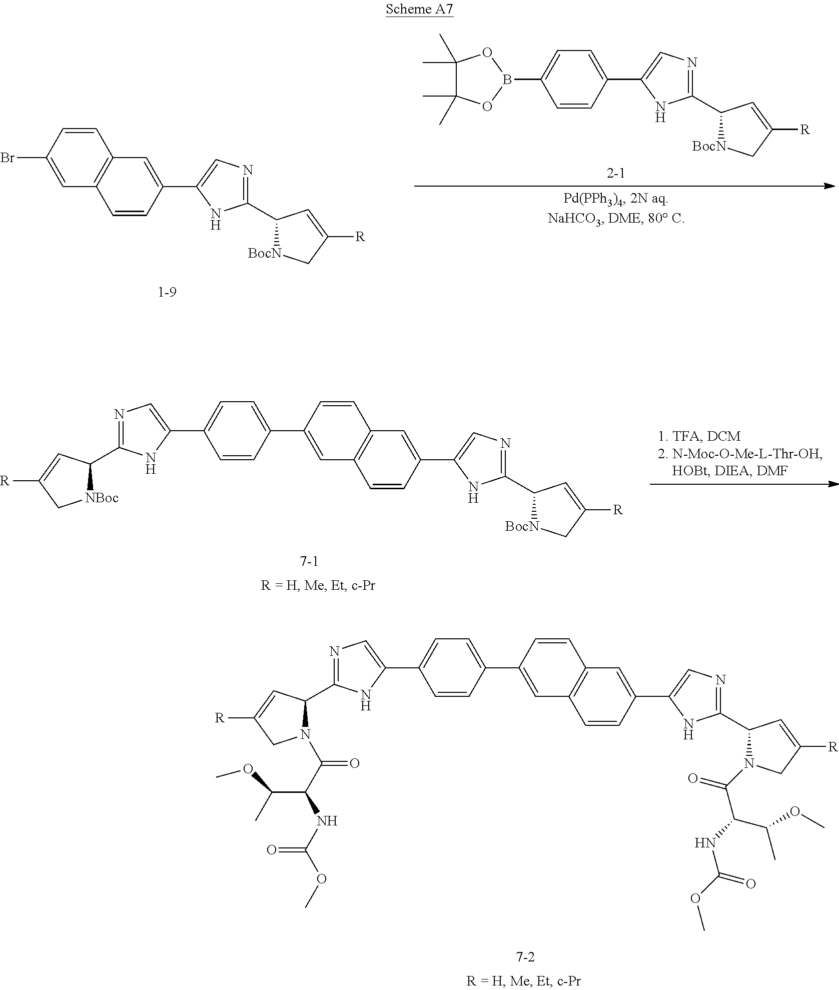

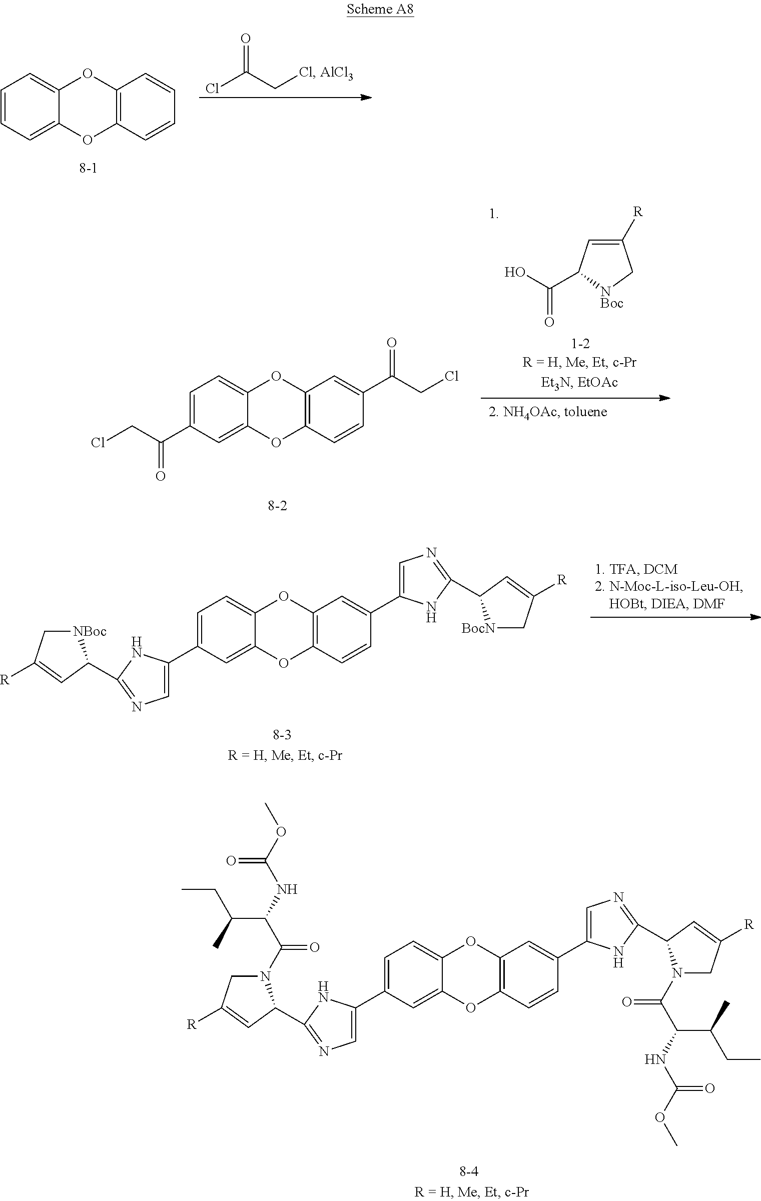

Example Compounds

Below are representative examples of the invention.

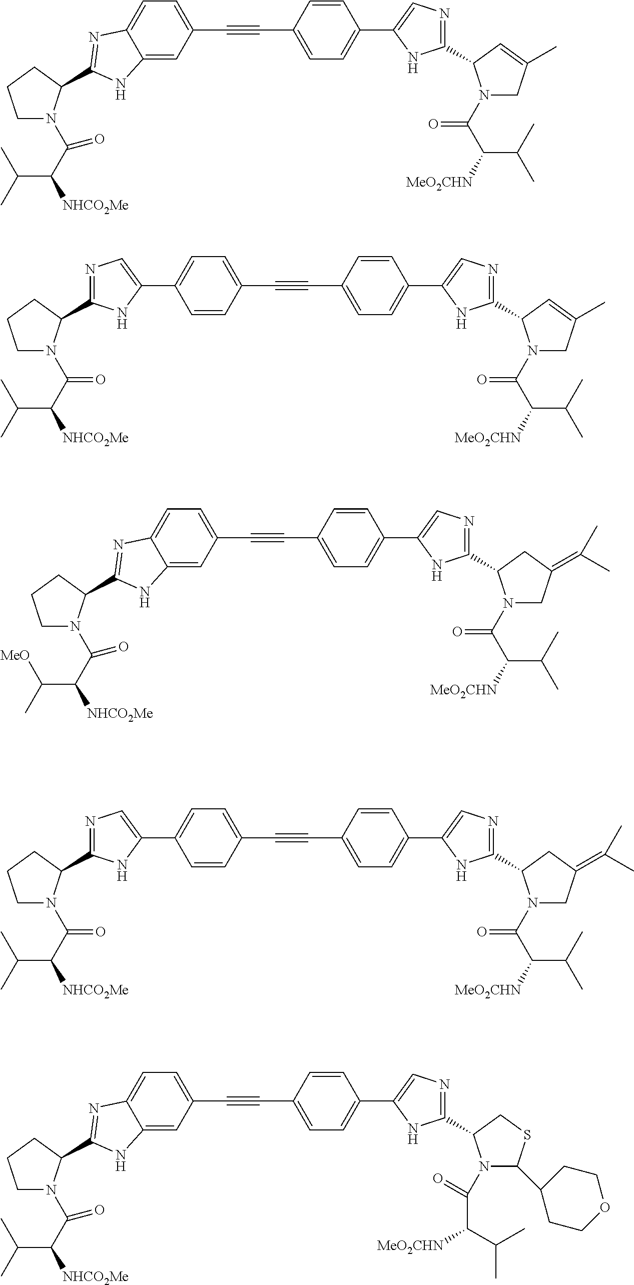

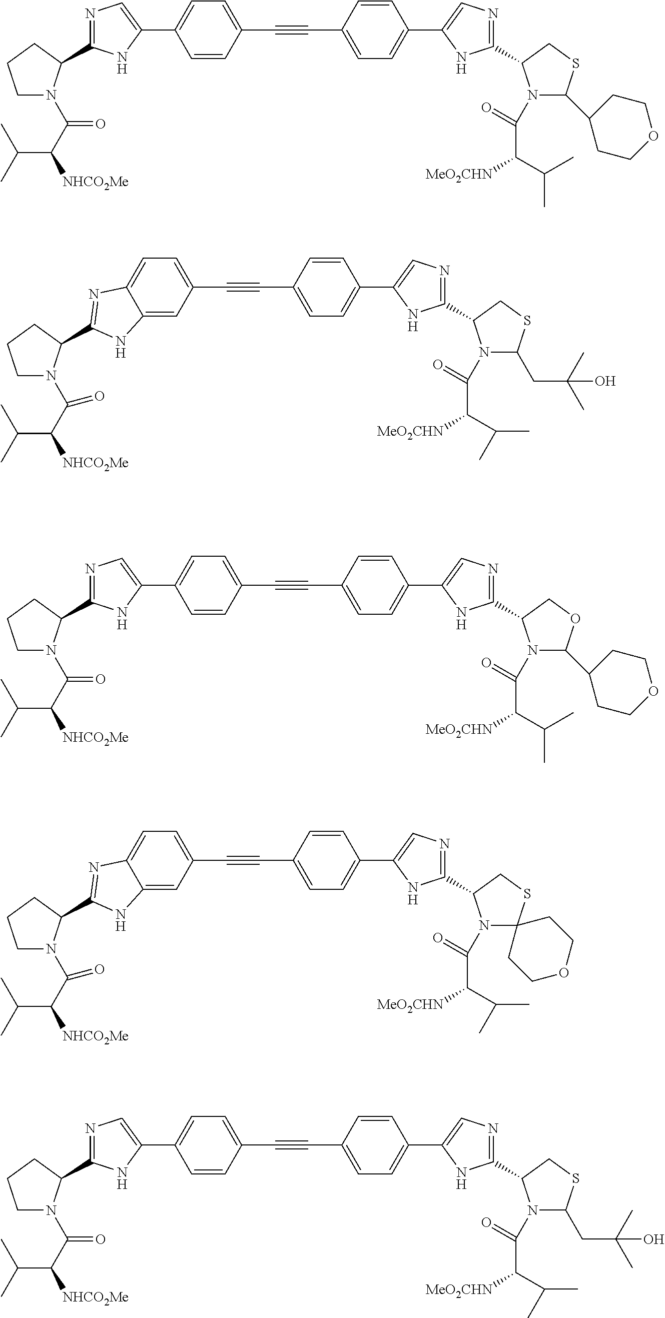

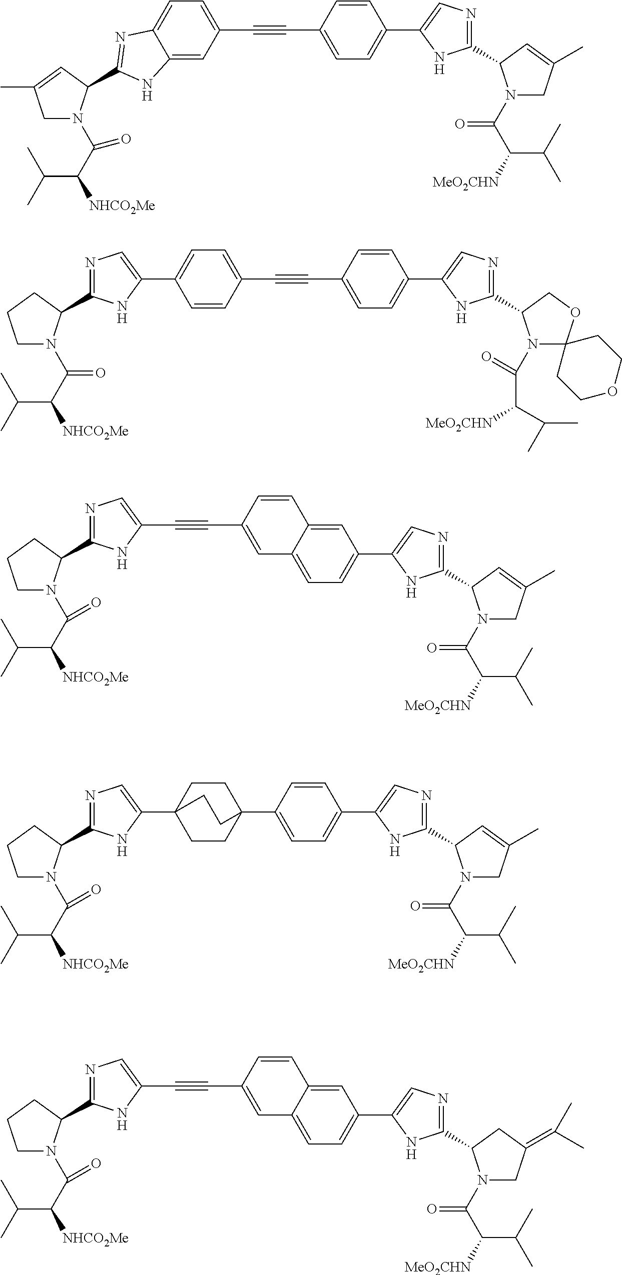

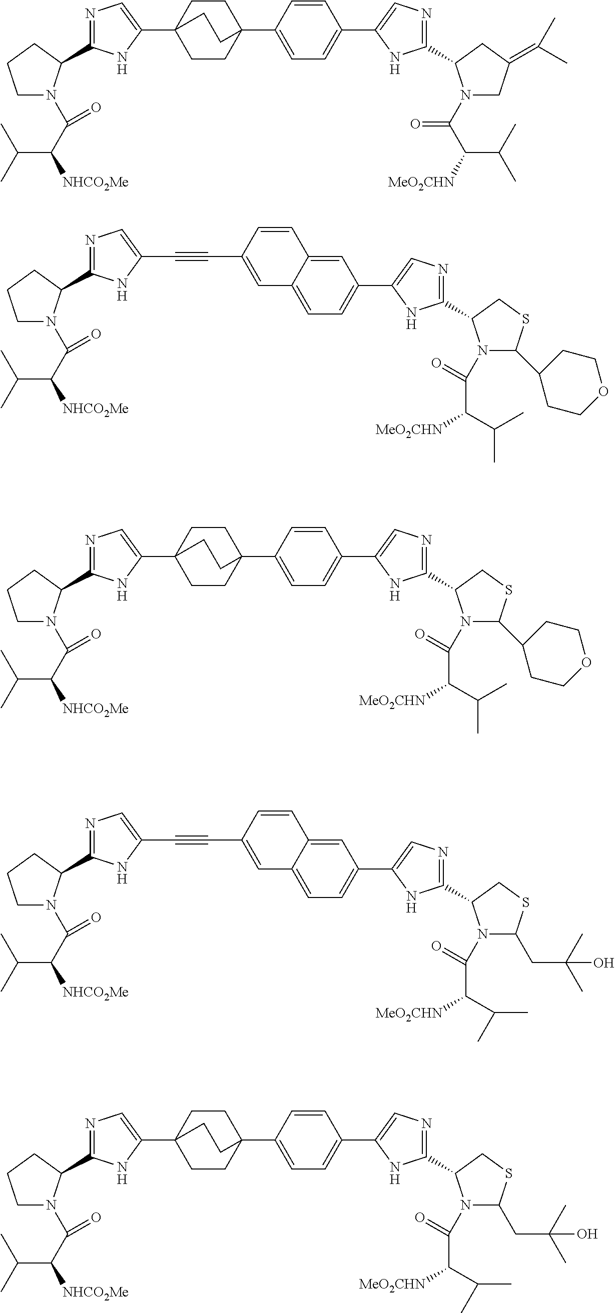

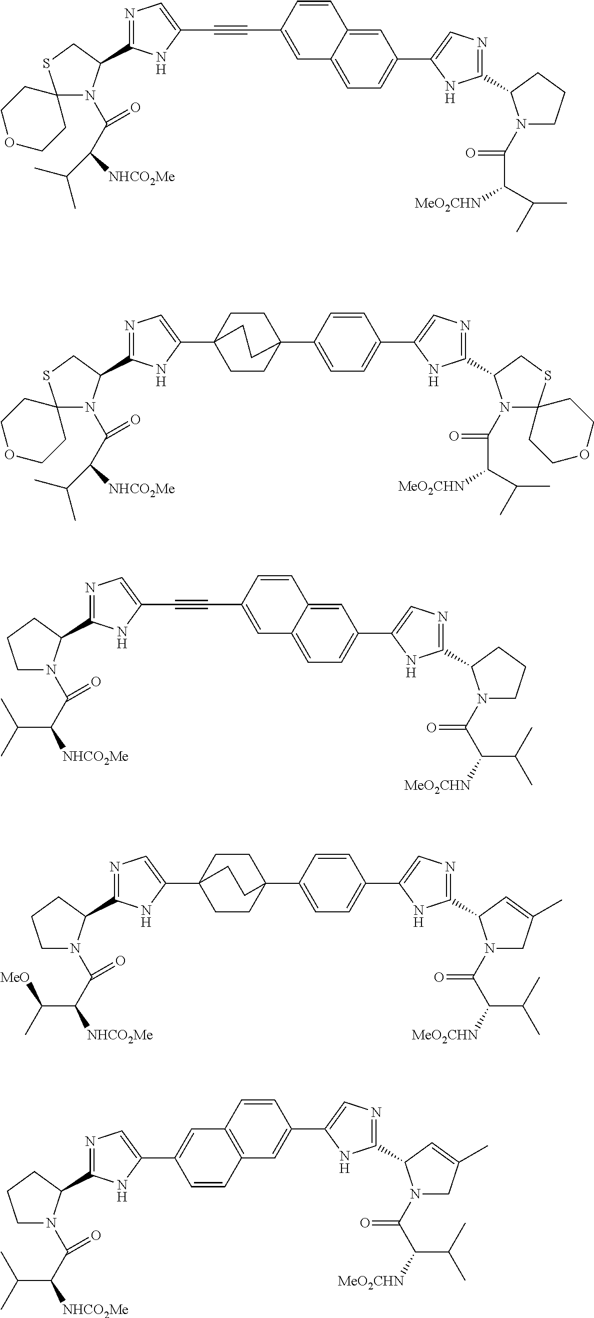

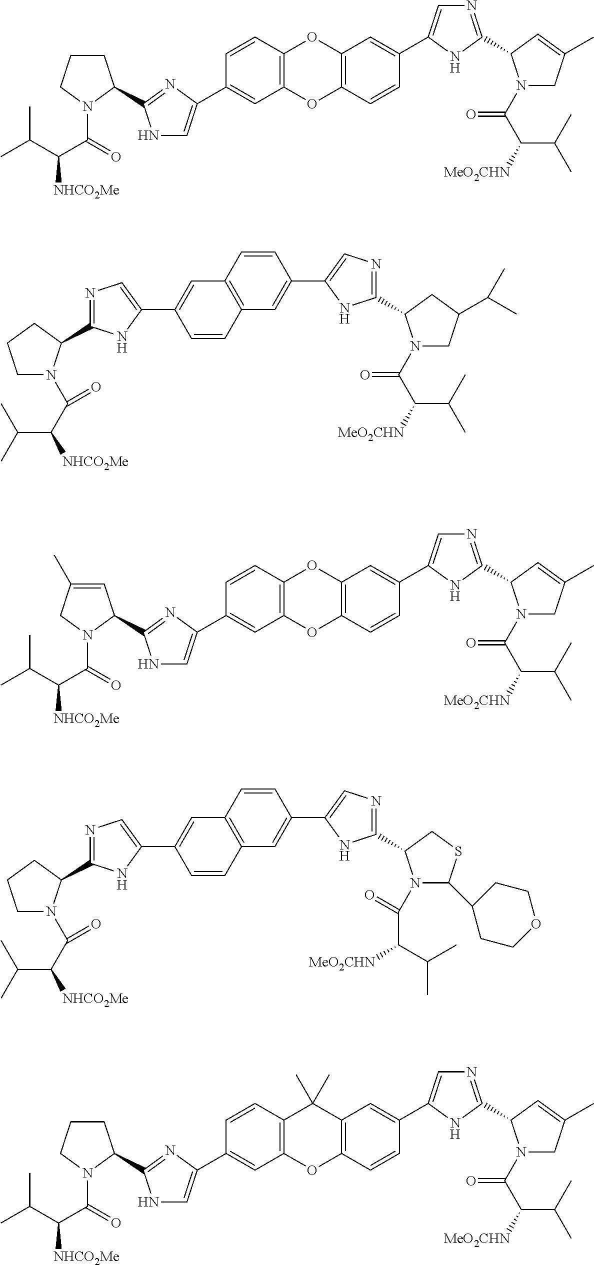

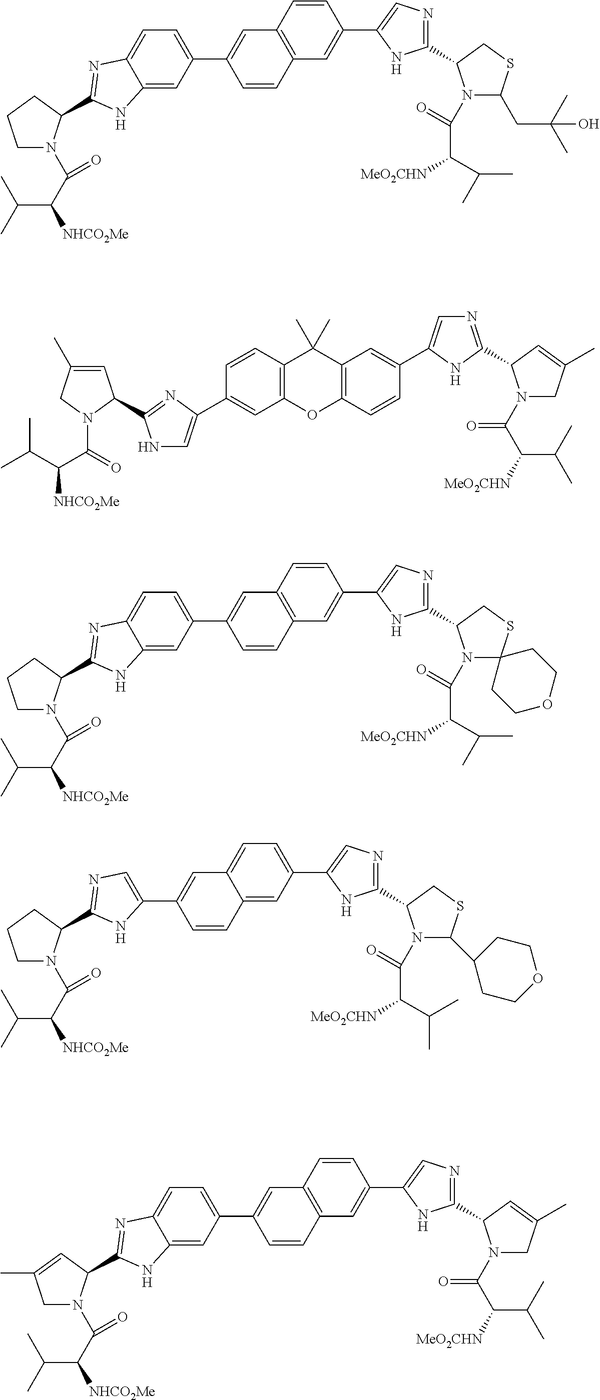

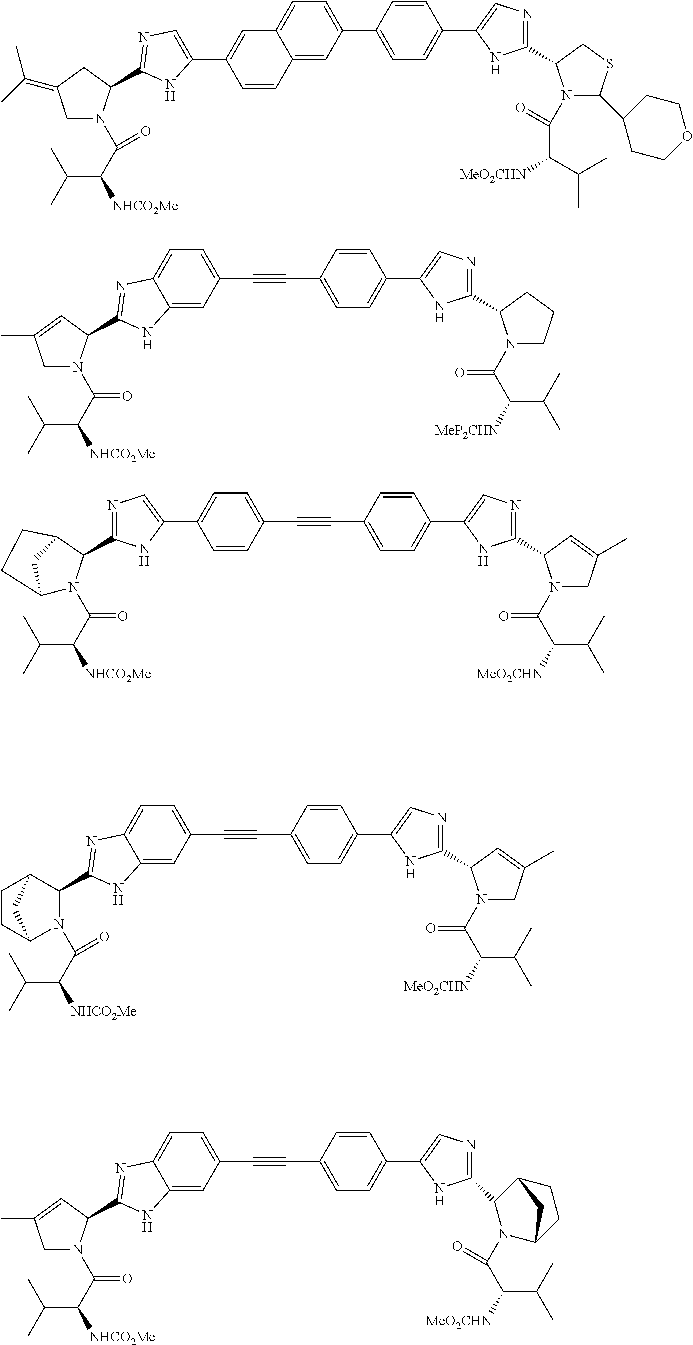

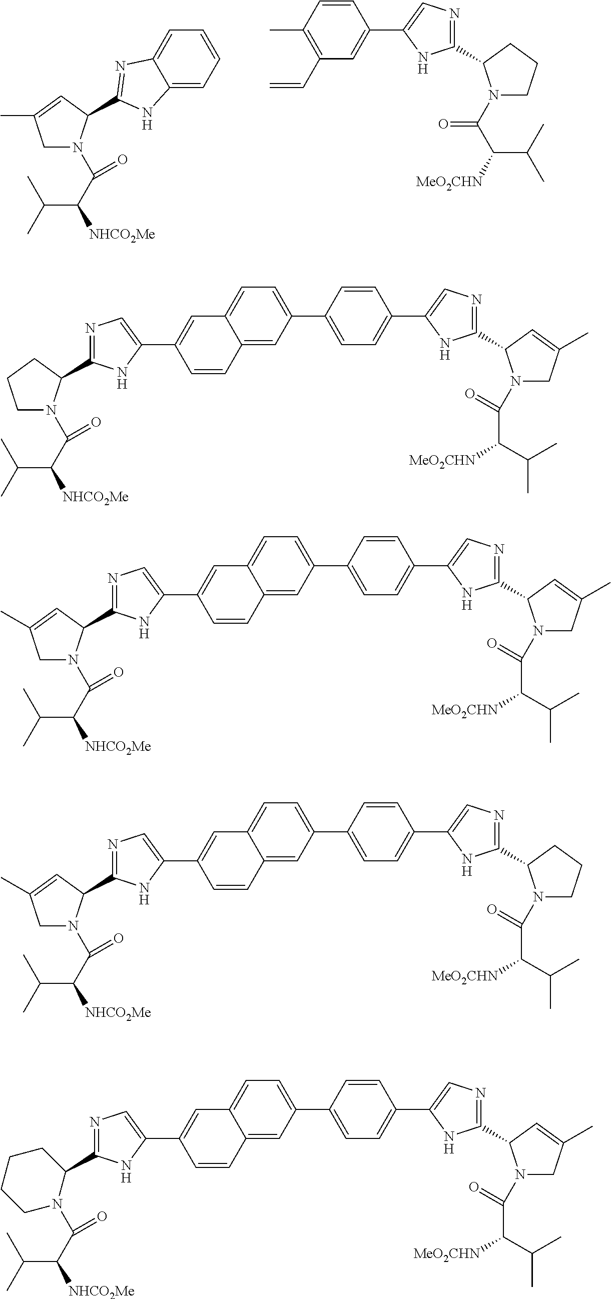

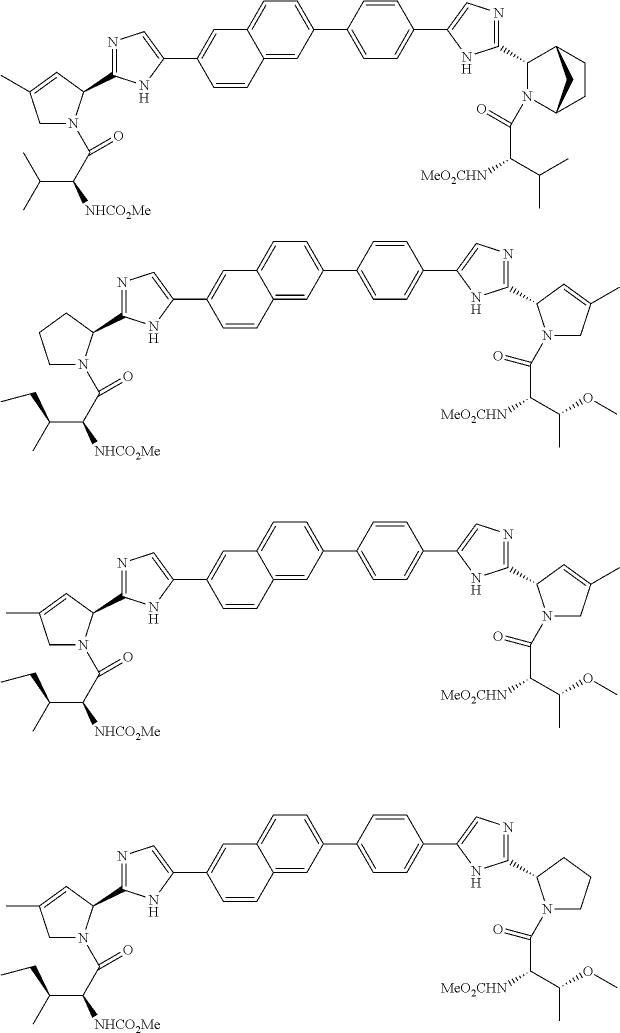

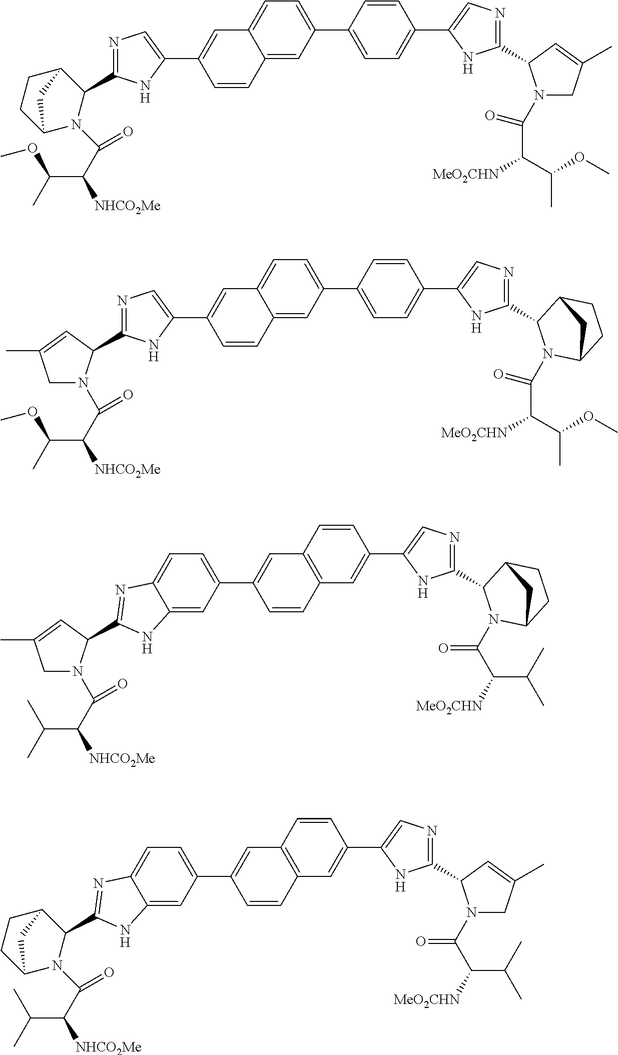

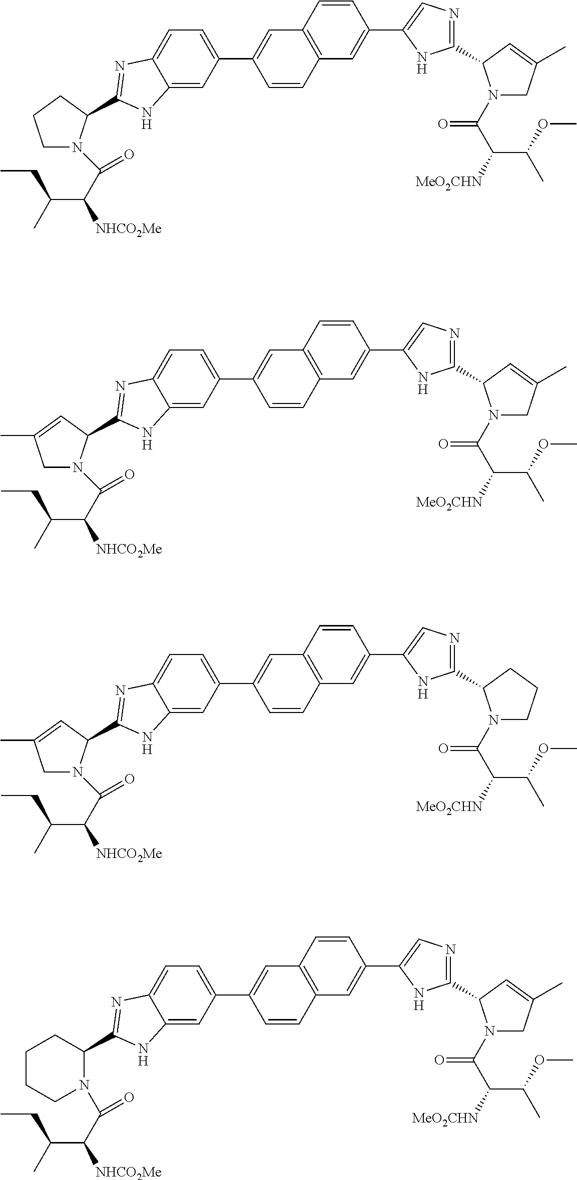

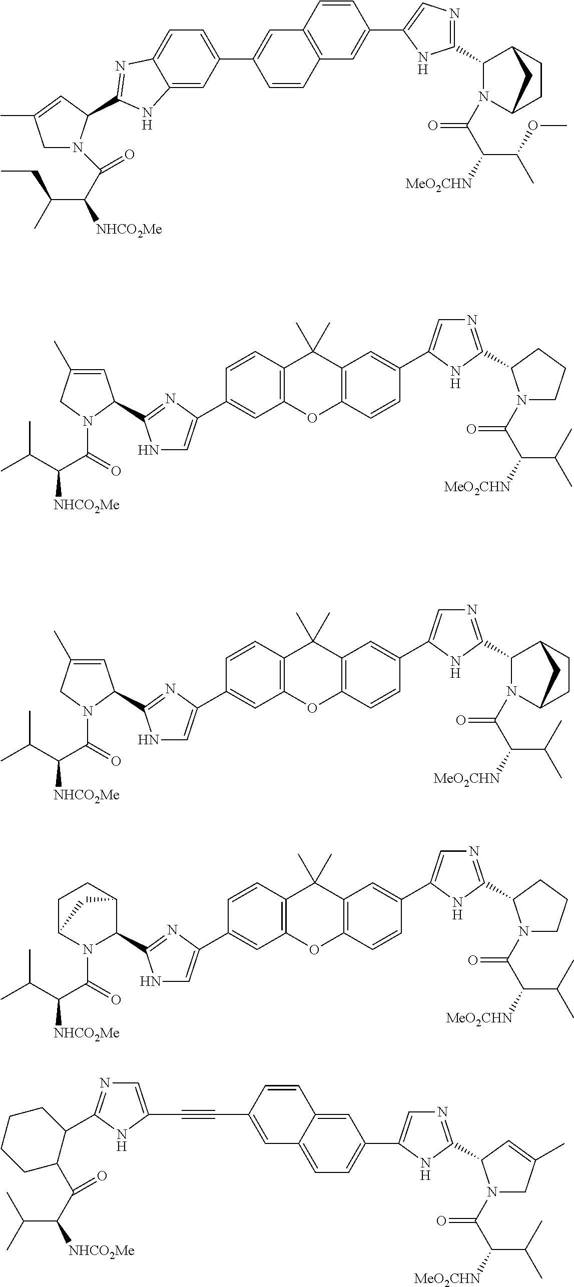

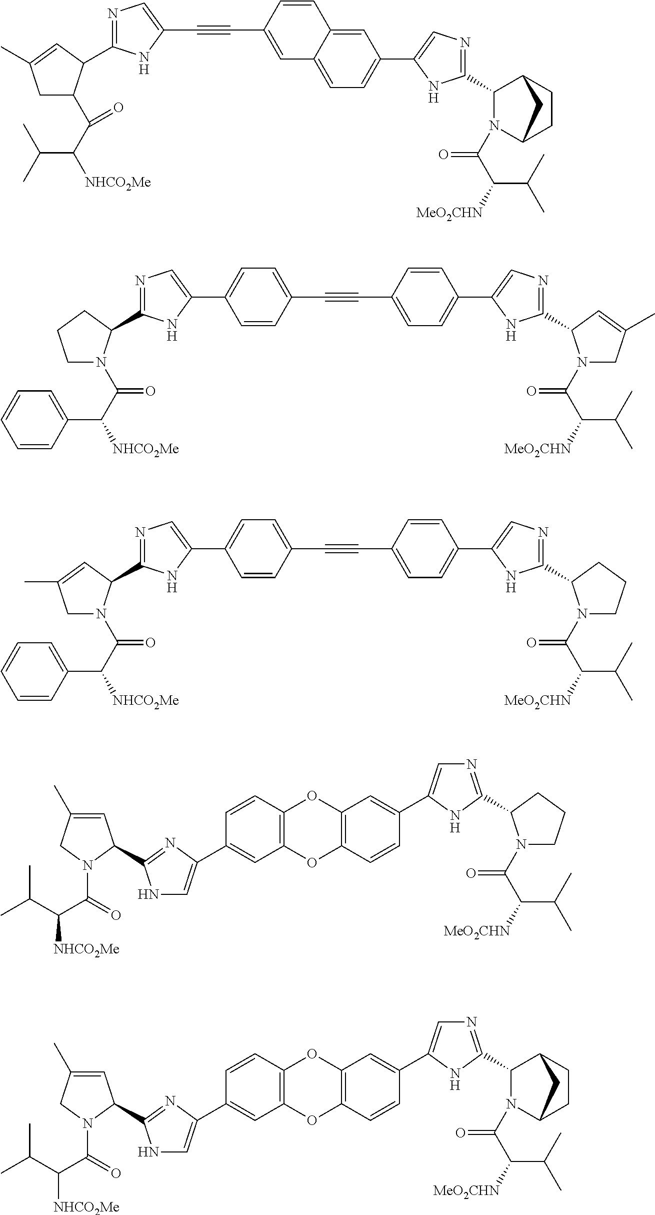

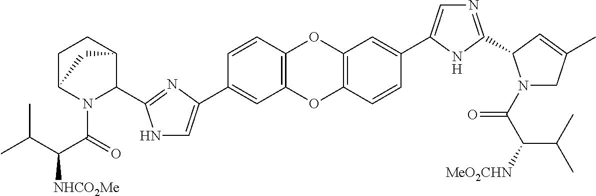

Biological Activity

Biological activity of the compounds of the invention was determined using an HCV replicon assay. The 1b_Huh-Luc/Neo-ET cell line persistently expressing a bicistronic genotype 1b replicon in Huh 7 cells was obtained from ReBLikon GMBH. This cell line was used to test compound inhibition using luciferase enzyme activity readout as a measurement of compound inhibition of replicon levels.

On Day 1 (the day after plating), each compound is added in triplicate to the cells. Plates incubated for 72 h prior to running the luciferase assay. Enzyme activity was measured using a Bright-Glo Kit (cat. number E2620) manufactured by Promega Corporation. The following equation was used to generate a percent control value for each compound.

% Control=(Average Compound Value/Average Control)*100

The EC₅₀ value was determined using GraphPad Prism and the following equation:

Y=Bottom+(Top−Bottom)/(1+10̂((Log IC50−X)*HillSlope))

EC₅₀ values of compounds are repeated several times in the replicon assay.

Synthesized compounds of the disclosed invention along with inhibitory activity and mass spectrometry results are illustrated in Table 1 below. The biological activity is indicated as being *, **, ***, or ****, which corresponds to EC₅₀ ranges of >1000 nM, 999 nM to 10 nM, 9.9 nM to 1 nM, or <1 nM respectively.

TABLE 1

| | | Inhibition | |
|---|---|---|---|
| Compound | | of HCV | MS |
| ID | Structure | genotype 1b | [M + H]+ |
| 101 | 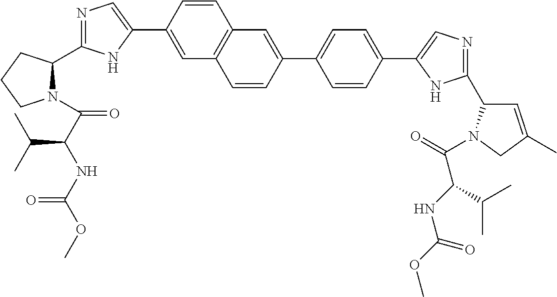 | **** | 801.4 |
| 102 | 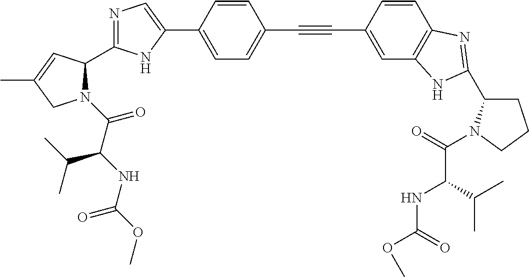 [SMILES: COC(=O)N[C@H](C(=O)N1CC(C)=C[C@H]1c1ncc(-c2ccc(C#Cc3ccc4nc([C@@H]5CCCN5C(=O)[C@@H](NC(=O)OC)C(C)C)[nH]c4c3)cc2)[nH]1)C(C)C] | **** | 749.4 |
| 103 | 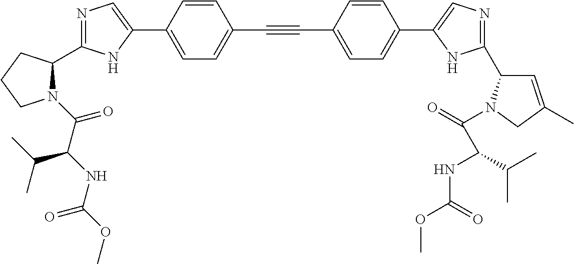 [SMILES: COC(=O)N[C@H](C(=O)N1CC(C)=C[C@H]1c1ncc(-c2ccc(C#Cc3ccc(-c4cnc([C@@H]5CCCN5C(=O)[C@@H](NC(=O)OC)C(C)C)[nH]4)cc3)cc2)[nH]1)C(C)C] | **** | 775.4 |
| 104 | 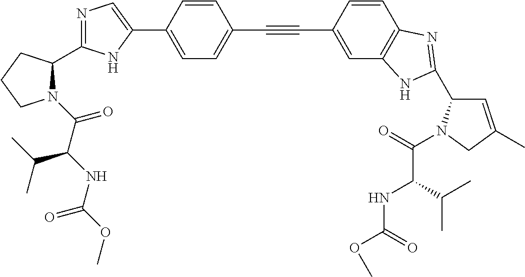 [SMILES: COC(=O)N[C@H](C(=O)N1CC(C)=C[C@H]1c1nc2ccc(C#Cc3ccc(-c4cnc([C@@H]5CCCN5C(=O)[C@@H](NC(=O)OC)C(C)C)[nH]4)cc3)cc2[nH]1)C(C)C] | **** | 749.4 |
| 105 | 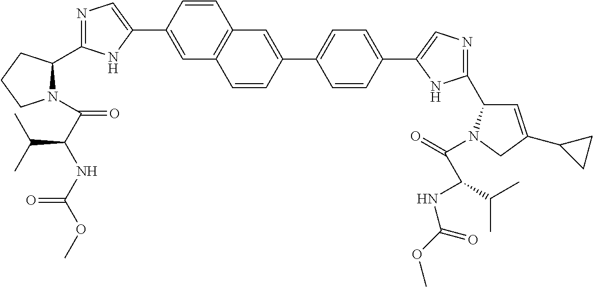 [SMILES: COC(=O)N[C@H](C(=O)N1CC(C2CC2)=C[C@H]1c1ncc(-c2ccc(-c3ccc4cc(-c5cnc([C@@H]6CCCN6C(=O)[C@@H](NC(=O)OC)C(C)C)[nH]5)ccc4c3)cc2)[nH]1)C(C)C] | **** | 827.4 |
| 106 | 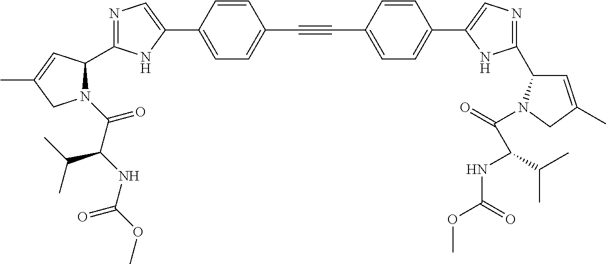 | **** | 775.4 |
| 107 | 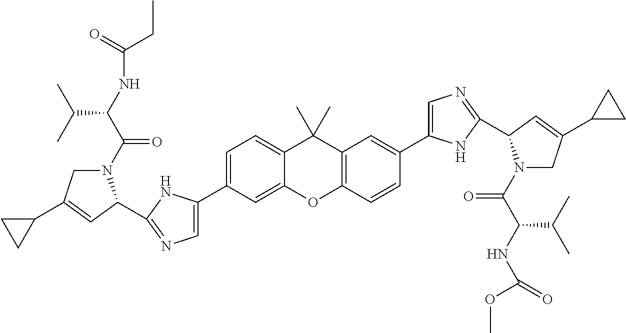 [SMILES: CCC(=O)N[C@H](C(=O)N1CC(C2CC2)=C[C@H]1c1ncc(-c2ccc3c(c2)Oc2ccc(-c4cnc([C@@H]5C=C(C6CC6)CN5C(=O)[C@@H](NC(=O)OC)C(C)C)[nH]4)cc2C3(C)C)[nH]1)C(C)C] | **** | 871.4 |
| 108 | 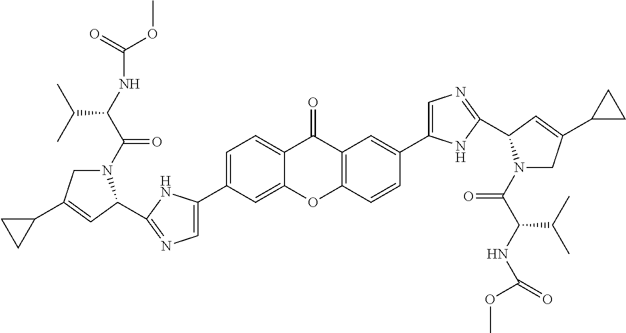 [SMILES: COC(=O)N[C@H](C(=O)N1CC(C2CC2)=C[C@H]1c1ncc(-c2ccc3c(=O)c4cc(-c5cnc([C@@H]6C=C(C7CC7)CN6C(=O)[C@@H](NC(=O)OC)C(C)C)[nH]5)ccc4oc3c2)[nH]1)C(C)C] | **** | 867.4 |
| 109 | 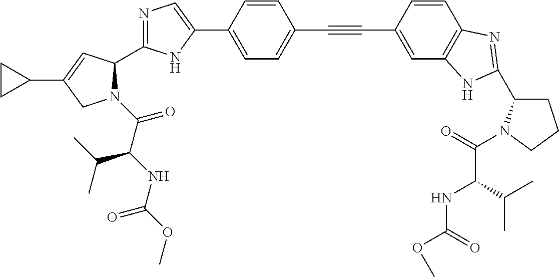 [SMILES: COC(=O)N[C@H](C(=O)N1CC(C2CC2)=C[C@H]1c1ncc(-c2ccc(C#Cc3ccc4nc([C@@H]5CCCN5C(=O)[C@@H](NC(=O)OC)C(C)C)[nH]c4c3)cc2)[nH]1)C(C)C] | **** | 775.4 |
| 110 | 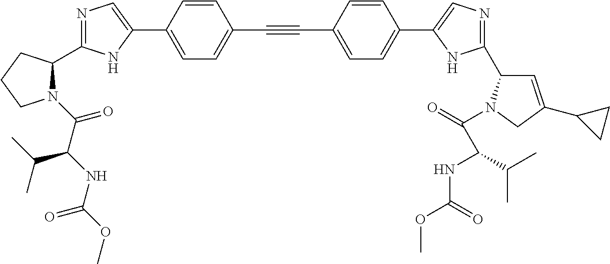 [SMILES: COC(=O)N[C@H](C(=O)N1CC(C2CC2)=C[C@H]1c1ncc(-c2ccc(C#Cc3ccc(-c4cnc([C@@H]5CCCN5C(=O)[C@@H](NC(=O)OC)C(C)C)[nH]4)cc3)cc2)[nH]1)C(C)C] | **** | 801.4 |
| 111 | 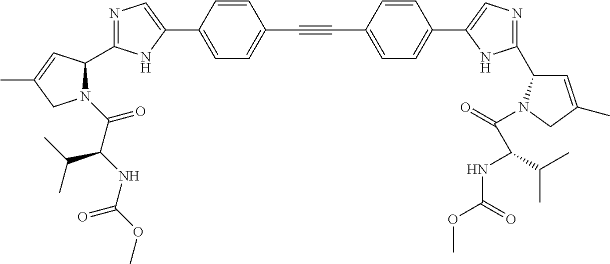 | **** | 787.4 |
| 112 | 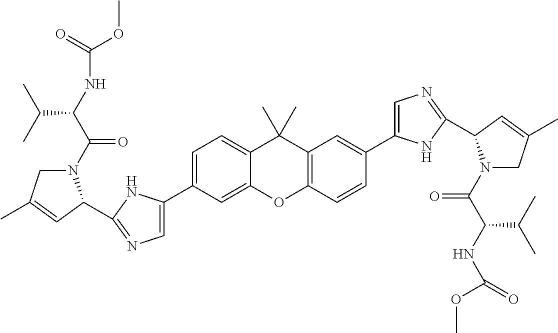 | **** | 819.4 |
| 113 | 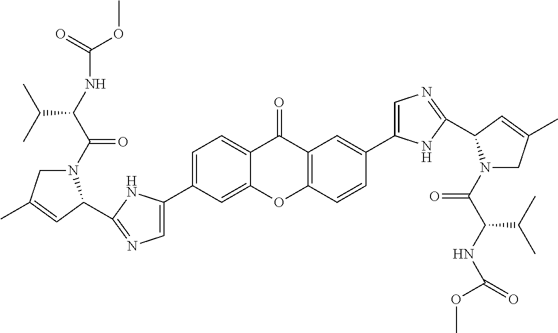 | **** | 805.4 |
| 114 | 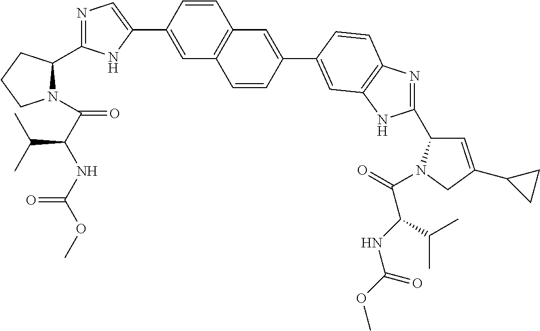 [SMILES: COC(=O)N[C@H](C(=O)N1CC(C2CC2)=C[C@H]1c1nc2ccc(-c3ccc4cc(-c5cnc([C@@H]6CCCN6C(=O)[C@@H](NC(=O)OC)C(C)C)[nH]5)ccc4c3)cc2[nH]1)C(C)C] | **** | 801.4 |
| 115 | 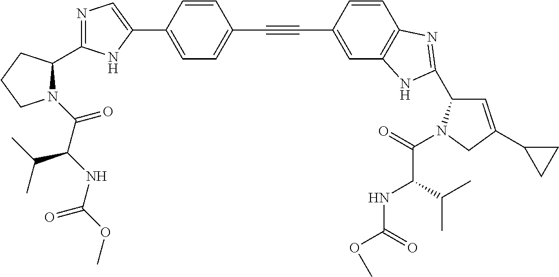 [SMILES: COC(=O)N[C@H](C(=O)N1CC(C2CC2)=C[C@H]1c1nc2ccc(C#Cc3ccc(-c4cnc([C@@H]5CCCN5C(=O)[C@@H](NC(=O)OC)C(C)C)[nH]4)cc3)cc2[nH]1)C(C)C] | **** | 775.4 |
| 116 | 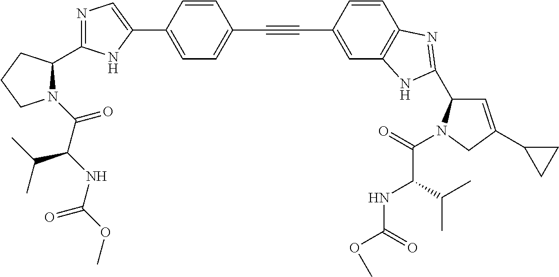 [SMILES: COC(=O)N[C@H](C(=O)N1CC(C2CC2)=C[C@@H]1c1nc2ccc(C#Cc3ccc(-c4cnc([C@@H]5CCCN5C(=O)[C@@H](NC(=O)OC)C(C)C)[nH]4)cc3)cc2[nH]1)C(C)C] | *** | 775.4 |
| 117 | 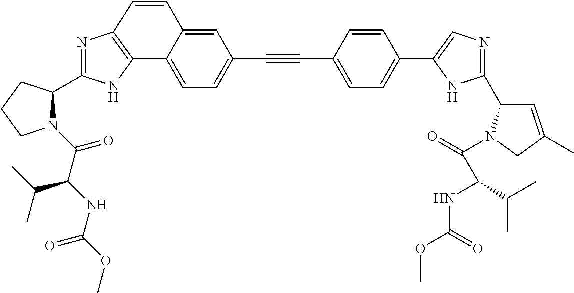 [SMILES: COC(=O)N[C@H](C(=O)N1CC(C)=C[C@H]1c1ncc(-c2ccc(C#Cc3ccc4c(ccc5nc([C@@H]6CCCN6C(=O)[C@@H](NC(=O)OC)C(C)C)[nH]c54)c3)cc2)[nH]1)C(C)C] | **** | 801.4 |
| 118 | 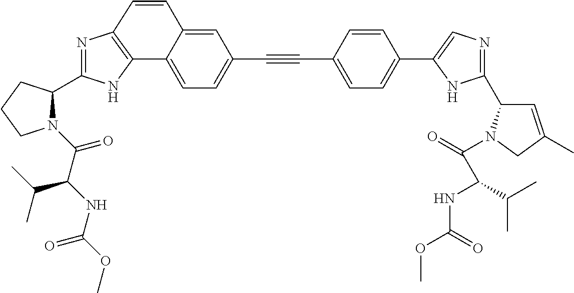 [SMILES: COC(=O)N[C@H](C(=O)N1CC(C)=C[C@H]1c1ncc(-c2ccc(C#Cc3ccc4c(ccc5nc([C@@H]6CCCN6C(=O)[C@@H](NC(=O)OC)C(C)C)[nH]c54)c3)cc2)[nH]1)C(C)C] | **** | 799.4 |
| 119 | 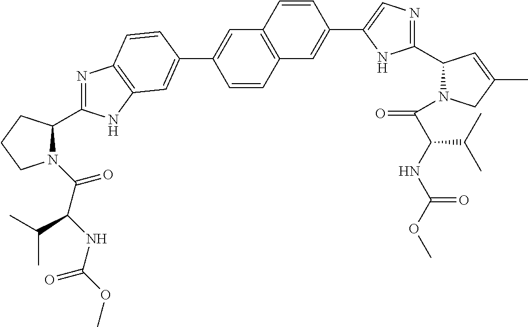 [SMILES: COC(=O)N[C@H](C(=O)N1CC(C)=C[C@H]1c1ncc(-c2ccc3cc(-c4ccc5nc([C@@H]6CCCN6C(=O)[C@@H](NC(=O)OC)C(C)C)[nH]c5c4)ccc3c2)[nH]1)C(C)C] | **** | 775.4 |
| 120 | 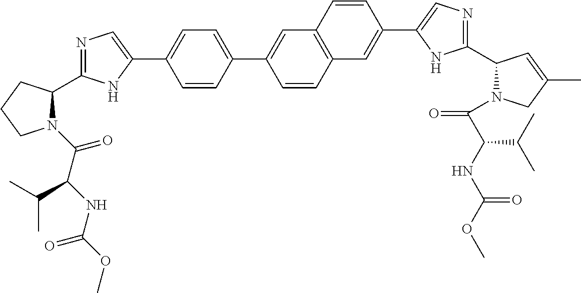 [SMILES: COC(=O)N[C@H](C(=O)N1CC(C)=C[C@H]1c1ncc(-c2ccc3cc(-c4ccc(-c5cnc([C@@H]6CCCN6C(=O)[C@@H](NC(=O)OC)C(C)C)[nH]5)cc4)ccc3c2)[nH]1)C(C)C] | **** | 801.4 |
| 121 | 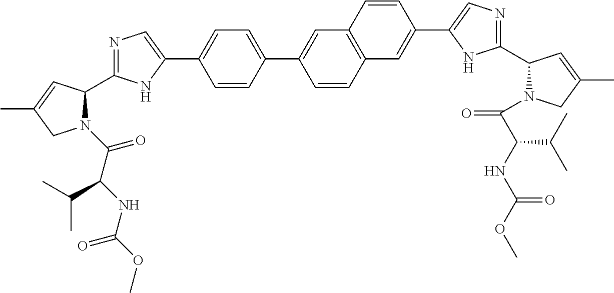 | **** | 813.4 |
| 122 | 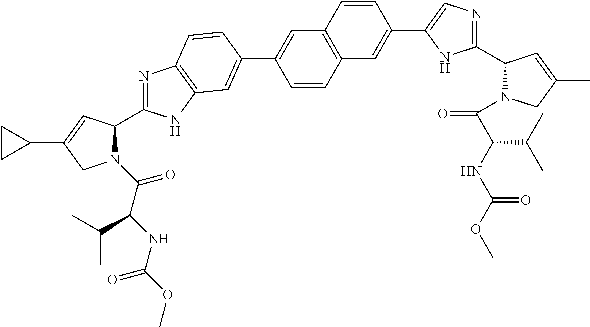 [SMILES: COC(=O)N[C@H](C(=O)N1CC(C)=C[C@H]1c1ncc(-c2ccc3cc(-c4ccc5nc([C@@H]6C=C(C7CC7)CN6C(=O)[C@@H](NC(=O)OC)C(C)C)[nH]c5c4)ccc3c2)[nH]1)C(C)C] | **** | 813.4 |
| 123 | 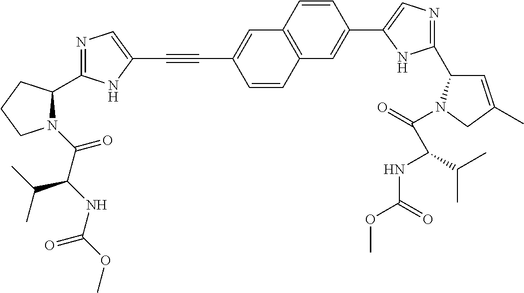 [SMILES: COC(=O)N[C@H](C(=O)N1CC(C)=C[C@H]1c1ncc(-c2ccc3cc(C#Cc4cnc([C@@H]5CCCN5C(=O)[C@@H](NC(=O)OC)C(C)C)[nH]4)ccc3c2)[nH]1)C(C)C] | **** | 749.4 |

Pharmaceutical Compositions

A fourth aspect of the invention provides a pharmaceutical composition comprising the compounds of the invention. In a first embodiment, the pharmaceutical composition further comprises one or more pharmaceutically acceptable excipients or vehicles, and optionally other therapeutic and/or prophylactic ingredients. Such excipients are known to those of skill in the art. The compounds of the present invention include, without limitation, basic compounds such as free bases and pharmaceutically acceptable salts of these compounds. A thorough discussion of pharmaceutically acceptable excipients and salts is available in Remington's Pharmaceutical Sciences, 18th Edition (Easton, Pa.: Mack Publishing Company, 1990).

Depending on the intended mode of administration, the pharmaceutical compositions may be in the form of solid, semi-solid or liquid dosage forms, such as, for example, tablets, suppositories, pills, capsules, powders, liquids, suspensions, creams, ointments, lotions or the like, preferably in unit dosage form suitable for single administration of a precise dosage. The compositions will include an effective amount of the selected drug in combination with a pharmaceutically acceptable carrier and, in addition, may include other pharmaceutical agents, adjuvants, diluents, buffers, etc.

The invention includes a pharmaceutical composition comprising a compound of the present invention including isomers, racemic or non-racemic mixtures of isomers, or pharmaceutically acceptable salts or solvates thereof together with one or more pharmaceutically acceptable carriers and optionally other therapeutic and/or prophylactic ingredients.

For solid compositions, conventional nontoxic solid carriers include, for example, pharmaceutical grades of mannitol, lactose, starch, magnesium stearate, sodium saccharin, talc, cellulose, glucose, sucrose, magnesium carbonate and the like.

For oral administration, the composition will generally take the form of a tablet, capsule, a softgel capsule nonaqueous solution, suspension or syrup. Tablets and capsules are preferred oral administration forms. Tablets and capsules for oral use will generally include one or more commonly used carriers such as lactose and corn starch. Lubricating agents, such as magnesium stearate, are also typically added. When liquid suspensions are used, the active agent may be combined with emulsifying and suspending agents. If desired, flavoring, coloring and/or sweetening agents may be added as well. Other optional components for incorporation into an oral formulation herein include, but are not limited to, preservatives, suspending agents, thickening agents and the like.

A fifth aspect of the invention provides use of the compounds of the invention in the manufacture of a medicament.

In a first embodiment of the fifth aspect the medicament is for the treatment of hepatitis C.

A sixth aspect of the invention provides a method of treating hepatitis C comprising administering to a subject in need thereof, a therapeutically effective amount of a compound of the invention, optionally in a pharmaceutical composition. A pharmaceutically or therapeutically effective amount of the composition will be delivered to the subject. The precise effective amount will vary from subject to subject and will depend upon the species, age, the subject's size and health, the nature and extent of the condition being treated, recommendations of the treating physician, and the therapeutics or combination of therapeutics selected for administration. Thus, the effective amount for a given situation can be determined by routine experimentation. The subject may be administered as many doses as is required to reduce and/or alleviate the signs, symptoms or causes of the disorder in question, or bring about any other desired alteration of a biological system. One of ordinary skill in the art of treating such diseases will be able, without undue experimentation and in reliance upon personal knowledge and the disclosure of this application, to ascertain a therapeutically effective amount of the compounds of this invention for a given disease.

Combination Therapy

The compounds of the present invention and their isomeric forms and pharmaceutically acceptable salts thereof are useful in treating and preventing HCV infection alone or when used in combination with other compounds targeting viral or cellular elements or functions involved in the HCV lifecycle. Classes of compounds useful in the invention may include, without limitation, all classes of HCV antivirals. For combination therapies, mechanistic classes of agents that may be useful when combined with the compounds of the present invention include, for example, nucleoside and non-nucleoside inhibitors of the HCV polymerase, protease inhibitors, helicase inhibitors, NS4B inhibitors and medicinal agents that functionally inhibit the internal ribosomal entry site (IRES) and other medicaments that inhibit HCV cell attachment or virus entry, HCV RNA translation, HCV RNA transcription, replication or HCV maturation, assembly or virus release. Specific compounds in these classes and useful in the invention include, but are not limited to, macrocyclic, heterocyclic and linear HCV protease inhibitors such as telaprevir (VX-950), boceprevir (SCH-503034), narlaprevir (SCH-900518), ITMN-191 (R-7227), TMC-435350 (a.k.a. TMC-435), MK-7009, BI-201335, BI-2061 (ciluprevir), BMS-650032, ACH-1625, ACH-1095 (HCV NS4A protease co-factor inhibitor), VX-500, VX-813, PHX-1766, PHX2054, IDX-136, IDX-316, ABT-450 EP-013420 (and congeners) and VBY-376; the Nucleosidic HCV polymerase (replicase) inhibitors useful in the invention include, but are not limited to, R7128, PSI-7851, IDX-184, IDX-102, R1479, UNX-08189, PSI-6130, PSI-938, PSI-879 and PSI-7977 and various other nucleoside and nucleotide analogs and HCV inhibitors including (but not limited to) those derived as 2′-C-methyl modified nucleos(t)ides, 4′-aza modified nucleos(t)ides, and 7′-deaza modified nucleos(t)ides. Non-nuclosidic HCV polymerase (replicase) inhibitors useful in the invention, include, but are not limited to, HCV-796, HCV-371, VCH-759, VCH-916, VCH-222, ANA-598, MK-3281, ABT-333, ABT-072, PF-00868554, BI-207127, GS-9190, A-837093, JKT-109, GL-59728 and GL-60667.

In addition, NS5A inhibitors of the present invention may be used in combination with cyclophyllin and immunophyllin antagonists (eg, without limitation, DEBIO compounds, NM-811 as well as cyclosporine and its derivatives), kinase inhibitors, inhibitors of heat shock proteins (e.g., HSP90 and HSP70), other immunomodulatory agents that may include, without limitation, interferons (-alpha, -beta, -omega, -gamma, -lambda or synthetic) such as Intron A™, Roferon-A™, Canferon-A300™, Advaferon™, Infergen™, Humoferon™, Sumiferon MP™, Alfaferone™, IFN-β™, Feron™ and the like; polyethylene glycol derivatized (pegylated) interferon compounds, such as PEG interferon-α-2a (Pegasys™), PEG interferon-α-2b (PEGIntron™), pegylated IFN-α-con1 and the like; long acting formulations and derivatizations of interferon compounds such as the albumin-fused interferon, Albuferon™, Locteron™ and the like; interferons with various types of controlled delivery systems (e.g. ITCA-638, omega-interferon delivered by the DUROS™ subcutaneous delivery system); compounds that stimulate the synthesis of interferon in cells, such as resiquimod and the like; interleukins; compounds that enhance the development of type 1 helper T cell response, such as SCV-07 and the like; TOLL-like receptor agonists such as CpG-10101 (actilon), isotorabine, ANA773 and the like; thymosin α-1; ANA-245 and ANA-246; histamine dihydrochloride; propagermanium; tetrachlorodecaoxide; ampligen; IMP-321; KRN-7000; antibodies, such as civacir, XTL-6865 and the like and prophylactic and therapeutic vaccines such as InnoVac C, HCV E1E2/MF59 and the like. In addition, any of the above-described methods involving administering an NS5A inhibitor, a Type I interferon receptor agonist (e.g., an IFN-α) and a Type II interferon receptor agonist (e.g., an IFN-γ) can be augmented by administration of an effective amount of a TNF-α antagonist. Exemplary, non-limiting TNF-α antagonists that are suitable for use in such combination therapies include ENBREL™, REMICADE™ and HUMIRA™.

In addition, NS5A inhibitors of the present invention may be used in combination with antiprotozoans and other antivirals thought to be effective in the treatment of HCV infection, such as, without limitation, the prodrug nitazoxanide. Nitazoxanide can be used as an agent in combination the compounds disclosed in this invention as well as in combination with other agents useful in treating HCV infection such as peginterferon alfa-2a and ribavarin (see, for example,_Rossignol, J F and Keeffe, E B, Future Microbiol. 3:539-545, 2008).

NS5A inhibitors of the present invention may also be used with alternative forms of interferons and pegylated interferons, ribavirin or its analogs (e.g., tarabavarin, levoviron), microRNA, small interfering RNA compounds (e.g., SIRPLEX-140-N and the like), nucleotide or nucleoside analogs, immunoglobulins, hepatoprotectants, anti-inflammatory agents and other inhibitors of NS5A. Inhibitors of other targets in the HCV lifecycle include NS3 helicase inhibitors; NS4A co-factor inhibitors; antisense oligonucleotide inhibitors, such as ISIS-14803, AVI-4065 and the like; vector-encoded short hairpin RNA (shRNA); HCV specific ribozymes such as heptazyme, RPI, 13919 and the like; entry inhibitors such as HepeX-C, HuMax-HepC and the like; alpha glucosidase inhibitors such as celgosivir, UT-231B and the like; KPE-02003002 and BIVN 401 and IMPDH inhibitors. Other illustrative HCV inhibitor compounds include those disclosed in the following publications: U.S. Pat. No. 5,807,876; U.S. Pat. No. 6,498,178; U.S. Pat. No. 6,344,465; U.S. Pat. No. 6,054,472; WO97/40028; WO98/40381; WO00/56331, WO 02/04425; WO 03/007945; WO 03/010141; WO 03/000254; WO 01/32153; WO 00/06529; WO 00/18231; WO 00/10573; WO 00/13708; WO 01/85172; WO 03/037893; WO 03/037894; WO 03/037895; WO 02/100851; WO 02/100846; EP 1256628; WO 99/01582; WO 00/09543; WO02/18369; WO98/17679, WO00/056331; WO 98/22496; WO 99/07734; WO 05/073216, WO 05/073195 and WO 08/021,927.

Additionally, combinations of, for example, ribavirin and interferon, may be administered as multiple combination therapy with at least one of the compounds of the present invention. The present invention is not limited to the aforementioned classes or compounds and contemplates known and new compounds and combinations of biologically active agents (see, Strader, D.B., Wright, T., Thomas, D.L. and Seeff, L.B., AASLD Practice Guidelines. 1-22, 2009 and Manns, M.P., Foster, G.R., Rockstroh, J.K., Zeuzem, S., Zoulim, F. and Houghton, M., Nature Reviews Drug Discovery. 6:991-1000, 2007, Pawlotsky, J-M., Chevaliez, S. and McHutchinson, J.G., Gastroenterology. 132:179-1998, 2007, Lindenbach, B.D. and Rice, C.M., Nature 436:933-938, 2005, Klebl, B.M., Kurtenbach, A., Salassidis, K., Daub, H. and Herget, T., Antiviral Chemistry & Chemotherapy. 16:69-90, 2005, Beaulieu, P.L., Current Opinion in Investigational Drugs. 8:614-634, 2007, Kim, S-J., Kim, J-H., Kim, Y-G., Lim, H-S. and Oh, W-J., The Journal of Biological Chemistry. 48:50031-50041, 2004, Okamoto, T., Nishimura, Y., Ichimura, T., Suzuki, K., Miyamura, T., Suzuki, T., Moriishi, K. and Matsuura, Y., The EMBO Journal. 1-11, 2006, Soriano, V., Peters, M.G. and Zeuzem, S. Clinical Infectious Diseases. 48:313-320, 2009, Huang, Z., Murray, M.G. and Secrist, J.A., Antiviral Research. 71:351-362, 2006 and Neyts, J., Antiviral Research. 71:363-371, 2006, each of which is incorporated by reference in their entirety herein). It is intended that combination therapies of the present invention include any chemically compatible combination of a compound of this inventive group with other compounds of the inventive group or other compounds outside of the inventive group, as long as the combination does not eliminate the anti-viral activity of the compound of this inventive group or the anti-viral activity of the pharmaceutical composition itself.

Combination therapy can be sequential, that is treatment with one agent first and then a second agent (for example, where each treatment comprises a different compound of the invention or where one treatment comprises a compound of the invention and the other comprises one or more biologically active agents) or it can be treatment with both agents at the same time (concurrently). Sequential therapy can include a reasonable time after the completion of the first therapy before beginning the second therapy. Treatment with both agents at the same time can be in the same daily dose or in separate doses. Combination therapy need not be limited to two agents and may include three or more agents. The dosages for both concurrent and sequential combination therapy will depend on absorption, distribution, metabolism and excretion rates of the components of the combination therapy as well as other factors known to one of skill in the art. Dosage values will also vary with the severity of the condition to be alleviated. It is to be further understood that for any particular subject, specific dosage regimens and schedules may be adjusted over time according to the individual's need and the professional judgment of the person administering or supervising the administration of the combination therapy.

All publications and patent applications cited in this specification are herein incorporated by reference as if each individual publication or patent application were specifically and individually indicated to be incorporated by reference.

Although the foregoing invention has been described in some detail by way of illustration and example for purposes of clarity of understanding, it will be readily apparent to one of ordinary skill in the art in light of the teachings of this invention that certain changes and modifications may be made thereto without departing from the spirit or scope of the invention as defined in the appended claims.

1. A compound of formula I,
D-A-B-A′-D′
or a pharmaceutically acceptable salt thereof,
wherein:
A and A′ are independently selected from the group consisting of 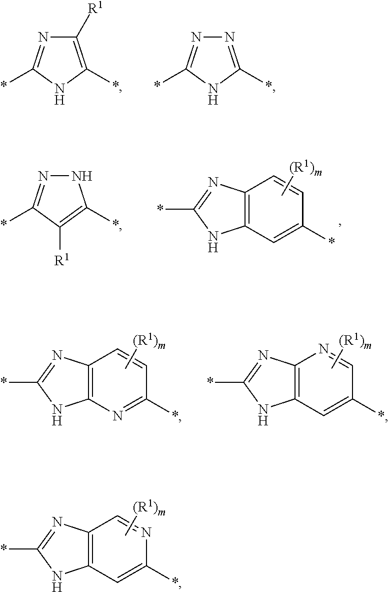
wherein
* indicates attachment points to the reminder of the compound,
R¹ is selected from the group consisting of C₁-C₄ alkyl, aryl, a halogen, —CN, —NO₂, —OR¹, —CF₃, —OCF₃, —OCHF₂, —CO₂R², —C(O)R³, —C(O)NR³R⁴, —NR³R⁴, —S(O)₂R², and —S(O)₂NR³R⁴,
m is 0, 1, or 3,
V is CH₂—CH₂—, —CH═CH—, —N═CH—, (CH₂)ₐ—N(R³)—(CH₂)_{b}— or (CH₂)ₐ—O—(CH₂)_{b}—, wherein a and b are independently 0, 1, 2, or 3 with the proviso that a and b are not both 0,
R², R³, and R⁴ are each independently chosen from the group consisting of hydrogen, C₁ to C₄ alkyl, C₁ to C₄ heteroalkyl, cycloalkyl, heterocycle, aryl, heteroaryl and aralkyl, and
wherein for each A and A′, B may be attached to either side of A and A′ so that in the example of A or A′ being 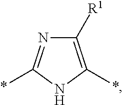
the A-B-A′ can be any of: 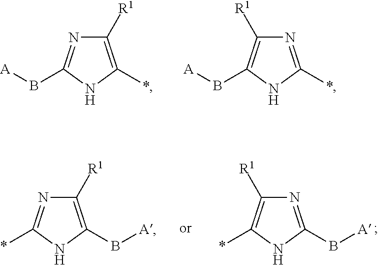
B is selected from the group consisting of a single bond, triple bond, [symbol] W, W [symbol] , [symbol] W [symbol] , W [symbol] W, W—W [symbol] , and W—W, wherein each W is independently selected from the group consisting of a cycloalkyl group, cycloalkenyl group, heterocyclic group, aryl group or heteroaryl group, with the proviso that when B is W—W, only one W is a six-member aromatic ring;
D is 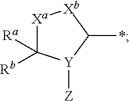
D′ is 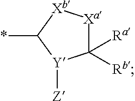
X^{a}—X^{b} and X^{a′}—X^{b′} are each independently selected from the group consisting of C₂ to C₆ alkyl, C₂ to C₆ alkenyl, C₂ to C₆ heteroalkyl, and C₂ to C₆ heteroalkenyl, wherein:
each hetero atom, if present, is independently N, O or S, and
either or both of X^{a}—X^{b} and X^{a′}—X^{b′}, together with the atoms to which they are attached, optionally form a 4- to 9-membered ring which may be cycloalkyl and heterocycle and which may optionally be fused to another 3-5 membered ring;
R^{a}, R^{b}, R^{a′} and R^{b′} are each independently hydrogen, C₁ to C₈ alkyl or C₁ to C₈ heteroalkyl, wherein:
each hetero atom, if present, is independently N, O or S,
R^{a} and R^{b} are optionally joined, together with the atom to which they are attached, to form a 3- to 6-membered ring, and
R^{a′} and R^{b′} are optionally joined, together with the atom to which they are attached, to form a 3- to 6-membered ring;
Y and Y′ are each independently N or CH; and
Z and Z′ are each independently selected from the group consisting of hydrogen, C₁ to C₈ alkyl, C₁ to C₈ heteroalkyl, cycloalkyl, heterocycle, aryl, heteroaryl, aralkyl, 1-3 amino acids,
—[U—(CR⁴₂)ₜ—NR⁵—(CR⁴₂)ₜ]ᵤ—U—(CR⁴₂)ₜ—NR⁷—(CR⁴₂)ₜ—R⁸,—U—(CR⁴₂)ₜ—R⁸ and
—[U—(CR⁴₂)ₜ—NR⁵—(CR⁴₂)ₜ]ᵤ—U—(CR⁴₂)ₜ—O—(CR⁴₂)ₜ—R⁸, wherein,
U is selected from the group consisting of —C(O)—, —C(S)— and —S(O)₂—,
each R⁴, R⁵ and R⁷ is independently selected from the group consisting of hydrogen, C₁ to C₈ alkyl, C₁ to C₈ heteroalkyl, cycloalkyl, heterocycle, aryl, heteroaryl and aralkyl,
R⁸ is selected from the group consisting of hydrogen, C₁ to C₈ alkyl, C₁ to C₈ heteroalkyl, cycloalkyl, heterocycle, aryl, heteroaryl, aralkyl, —C(O)—R⁸¹, —C(S)—R⁸¹, —C(O)—O—R⁸¹, —C(O)—N—R⁸¹₂, —S(O)₂—R⁸¹ and —S(O)₂—N—R⁸¹₂, wherein each R⁸¹ is independently chosen from the group consisting of hydrogen, C₁ to C₈ alkyl, C₁ to C₈ heteroalkyl, cycloalkyl, heterocycle, aryl, heteroaryl and aralkyl,
optionally, R⁷ and R⁸ together form a 4-7 membered ring,
each t is independently 0, 1, 2, 3, or 4, and
u is 0, 1, or 2.

2. The compound of claim 1 wherein A and A′ are selected from the group consisting of:

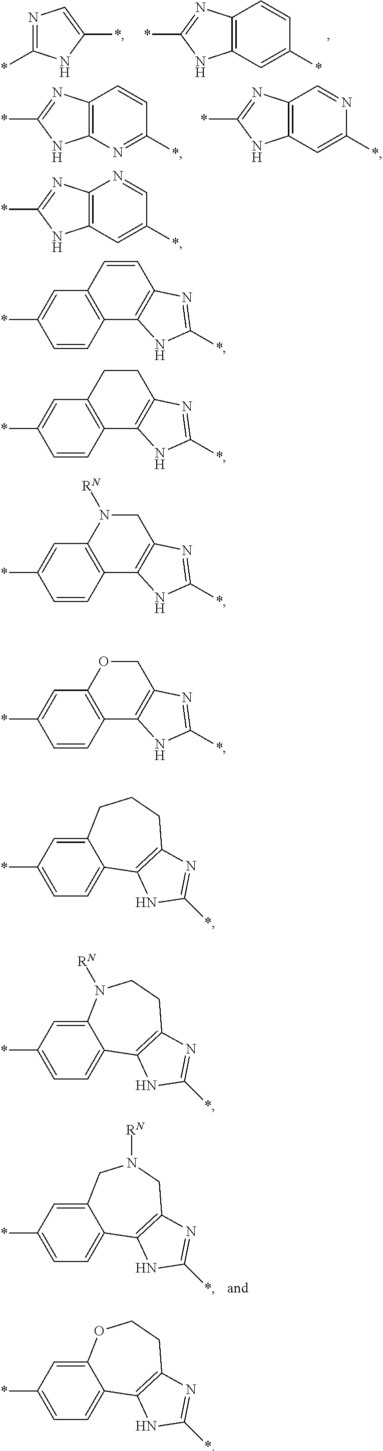

3. The compound of claim 1, wherein D is independently selected from group 1 and group 2 wherein:
Group 1 consists of 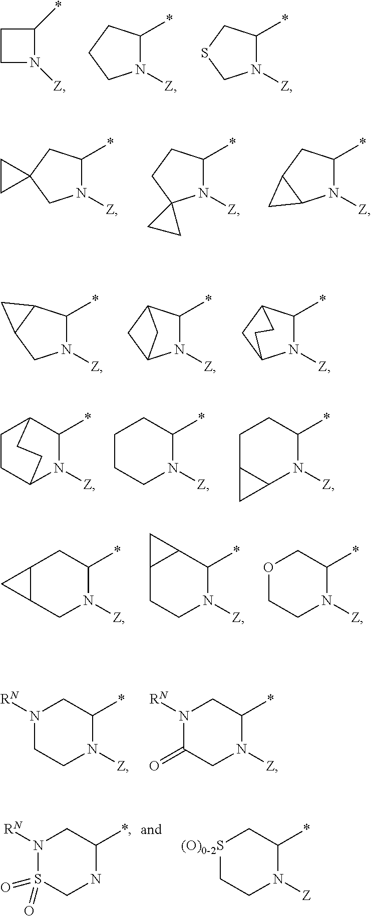
wherein R^{N} is independently selected from the group consisting of hydrogen, —OH, C₁ to C₁₂ alkyl, C₁ to C₁₂ heteroalkyl, cycloalkyl, heterocycle, aryl, heteroaryl, aralkyl, alkoxy, alkoxycarbonyl, alkanoyl, carbamoyl, substituted sulfonyl, sulfonate and sulfonamide; and
Group 2 consists of 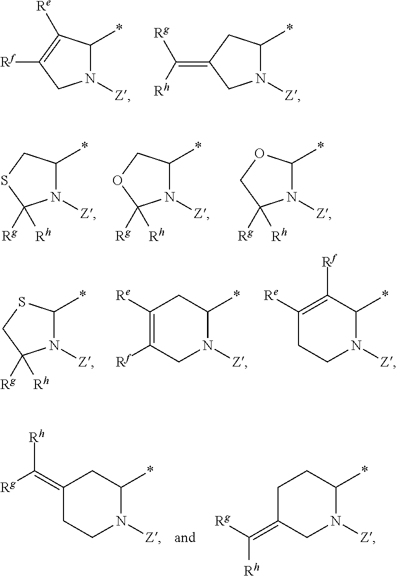
wherein R^{e}, R^{f}, R^{g}, and R^{h} are each independently hydrogen, C₁ to C₈ alkyl or C₁ to C₈ heteroalkyl, each hetero atom, if present, is independently N, O or S; R^{e} and R^{f} are optionally joined, together with the atom to which they are attached, to form a 5- to 8-membered ring, and R^{g} and R^{h} are optionally joined, together with the atom to which they are attached, to form a 3- to 8-membered ring.

4. The compound of claim 1, wherein D′ is independently selected from group 1′ and group 2′ wherein:
Group 1′ consists of 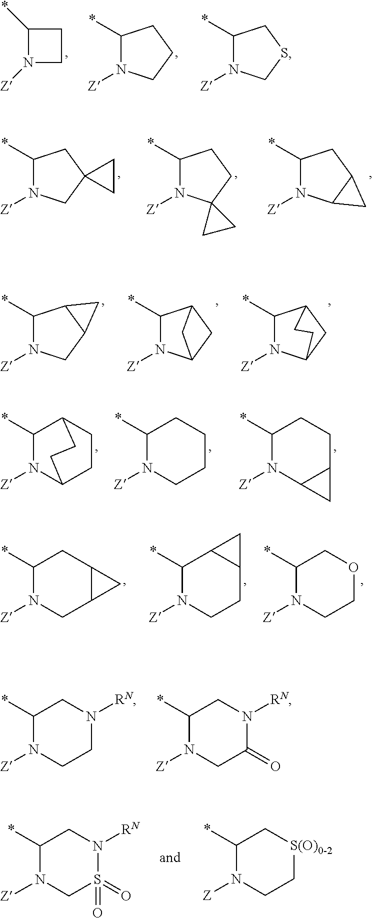
wherein R^{N} is independently selected from the group consisting of hydrogen, —OH, C₁ to C₁₂ alkyl, C₁ to C₁₂ heteroalkyl, cycloalkyl, heterocycle, aryl, heteroaryl, aralkyl, alkoxy, alkoxycarbonyl, alkanoyl, carbamoyl, substituted sulfonyl, sulfonate and sulfonamide; and
Group 2′ consists of 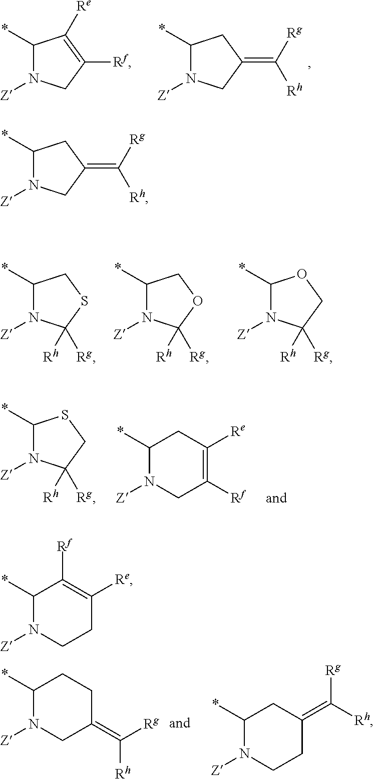
wherein R^{e}, R^{f}, R^{g}, and R^{h} are each independently hydrogen, C₁ to C₈ alkyl or C₁ to C₈ heteroalkyl, each hetero atom, if present, is independently N, O or S; R^{e} and R^{f} are optionally joined, together with the atom to which they are attached, to form a 5- to 8-membered ring, and R^{g} and R^{h} are optionally joined, together with the atom to which they are attached, to form a 3- to 8-membered ring.

5. The compound of claim 1, wherein if D is selected from Group 1, D′ is selected from Group 2′.

6. The compound of claim 1, wherein if D′ is selected from Group 1′, D is selected from Group 2.

7. The compound of claim 1, wherein A-B-A′ is selected from the group consisting of: 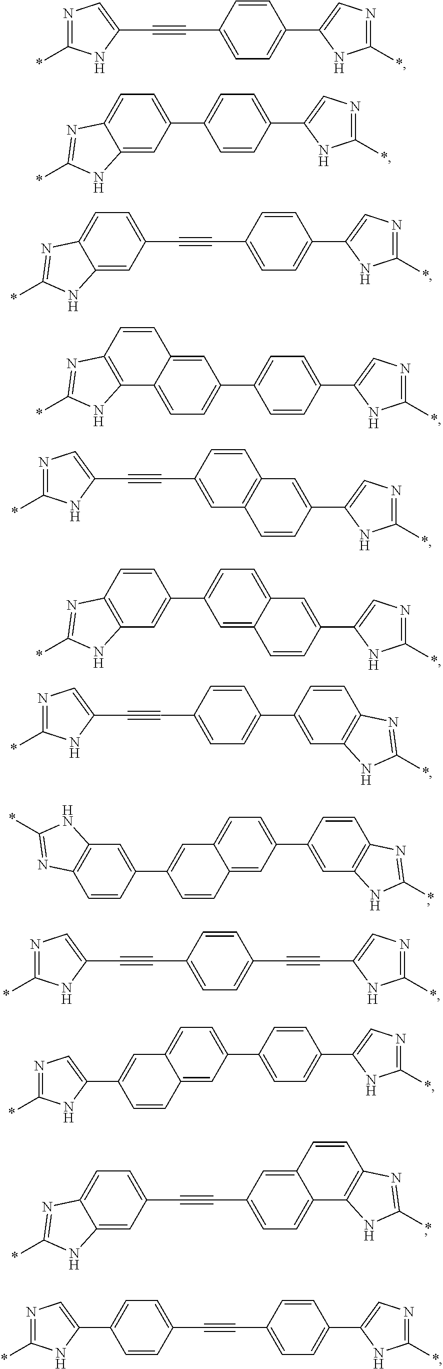 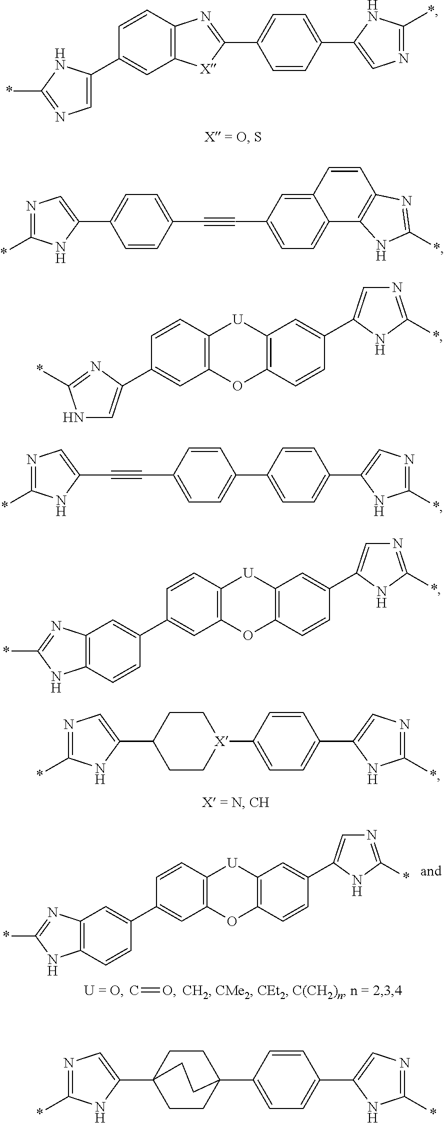
wherein * indicates attachment points to the reminder of the compound.

8. The compound of claim 1, wherein one or both of Y and Y′ are —N—.

9. The compound of claim 1, wherein Z and Z′ are each 1-3 amino acids.

10. The compound of claim 9 wherein the amino acids are all in the D or all in the L configuration.

11. The compound of claim 1, wherein Z and Z′ are each independently selected from the group consisting of
—[U—(CR⁴₂)ₜ—NR⁵—(CR⁴₂)ₜ]^{u}—U—(CR⁴₂)ₜ—NR⁷—(CR⁴₂)ₜ—R⁸,
—U—(CR⁴₂)ₜ—R⁸ and —[U—(CR⁴₂)ₜ—NR⁵—(CR⁴₂)ₜ]ᵤ—U—(CR⁴₂)ₜ—O—(CR⁴₂)ₜ—R⁸,
—[U—(CR⁴₂)ₜ—NR⁵—(CR⁴₂)ₜ]ᵤ—U—(CR⁴₂)ₜ—NR⁷—(CR⁴₂)ₜ—R⁸,
—U—(CR⁴₂)ₜ—NR⁵—(CR⁴₂)ₜ—U—CR⁴₂)ₜ—NR⁷—(CR⁴₂)ₜ—R⁸
—U—(CR⁴₂)ₜ—NR⁷—(CR⁴₂)ₜ—R⁸,
—[C(O)—(CR⁴₂)ₜ—NR⁵—(CR⁴₂]—U—(CR⁴₂)ₜ—NR⁷—(CR⁴₂)ₜ—R⁸,
—C(O)—(CR⁴₂)ₜ—NR⁵—(CR⁴₂)ₜ—U—(CR⁴₂)ₜ—NR⁷—(CR⁴₂)ₜ—R⁸,
—[C(O)—(CR⁴₂)ₜ—NR⁵—(CR⁴₂)ₜ]ᵤ—C(O)—(CR⁴₂)ₜ—NR⁷—(CR⁴₂)ₜ—R⁸,
—C(O)—(CR⁴₂)ₜ—NR⁵—(CR⁴₂)ₜ—C(O)—(CR⁴₂)ₜ—NR⁷—(CR⁴₂)ₜ—R⁸,
—C(O)—(CR⁴₂)ₜ—NR⁷—(CR⁴₂)ₜ—R⁸,
—C(O)—(CR⁴₂)ₙ—NR⁷—(CR⁴₂)ₙ—C(O)—R⁸¹,
—C(O)—(CR⁴₂)ₙ—NR⁷—C(O)—R⁸¹,
—C(O)—(CR⁴₂)ₙ—NR⁷—(CR⁴₂)ₙ—C(O)—O—R⁸¹,
—C(O)—(CR⁴₂)ₙ—NR⁷—C(O)—O—R⁸¹,
—U—(CR⁴₂)ₜ—R⁸,
—C(O)—(CR⁴₂)ₜ—R⁸,
—[U—(CR⁴₂)ₜ—NR⁵—(CR⁴₂)ₜ]ᵤ—U—(CR⁴₂)ₜ—O—(CR⁴₂)ₜ—R⁸,
—U—(CR⁴₂)ₜ—NR⁵—(CR⁴₂)ₜ—U—(CR⁴₂)ₜ—O—(CR⁴₂)ₜ—R⁸,
—C(O)—(CR⁴₂)ₜ—NR⁵—(CR⁴₂)ₜ—C(O)—(CR⁴₂)ₜ—O—(CR⁴₂)ₜ—R⁸,
—U—(CR⁴₂)ₜ—O—(CR⁴₂)ₜ—R⁸,
—C(O)—(CR⁴₂)ₜ—O—(CR⁴₂)ₜ—R⁸, and
—C(O)—(CR⁴₂)ₙ—NR⁷—R⁸ wherein R⁷ and R⁸ to ether form a 4-7 membered ring.

12-30. (canceled)

31. The compound of claim 1, wherein the compound is selected from the group consisting of: 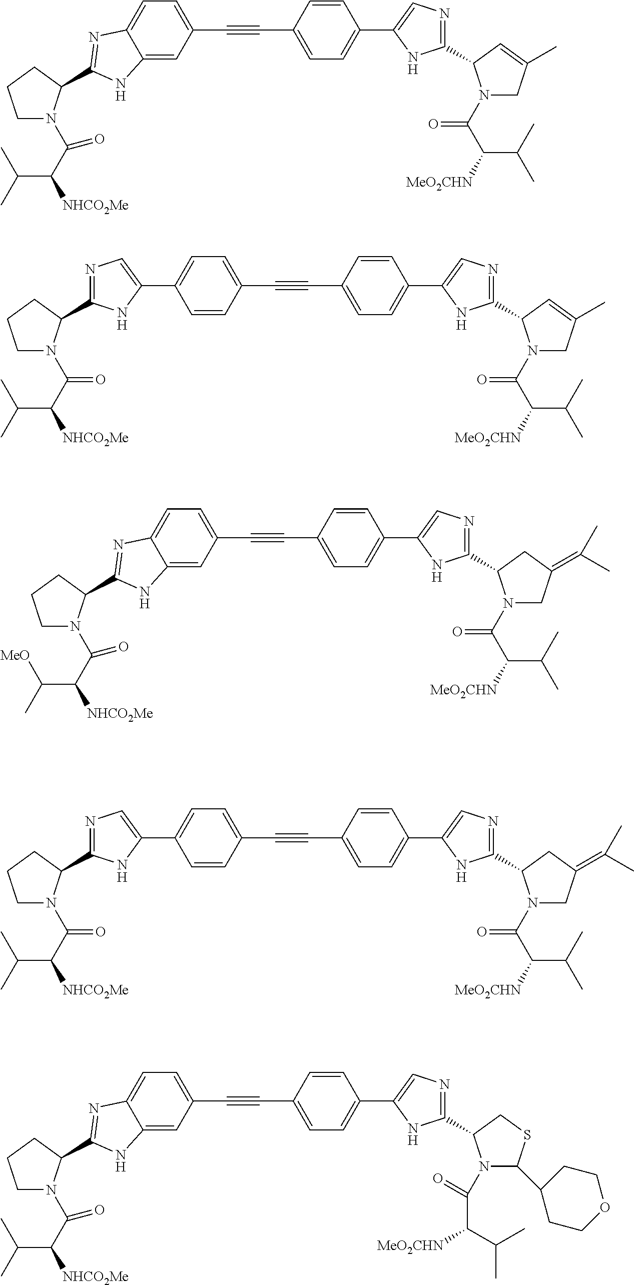 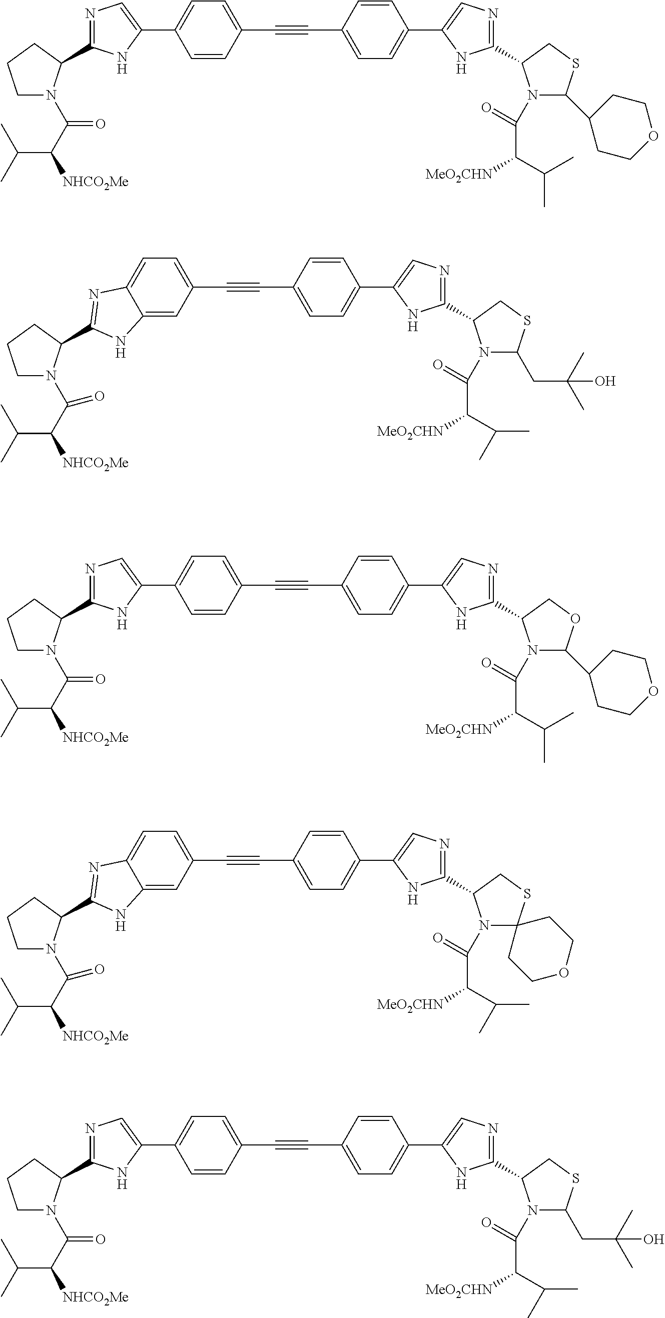 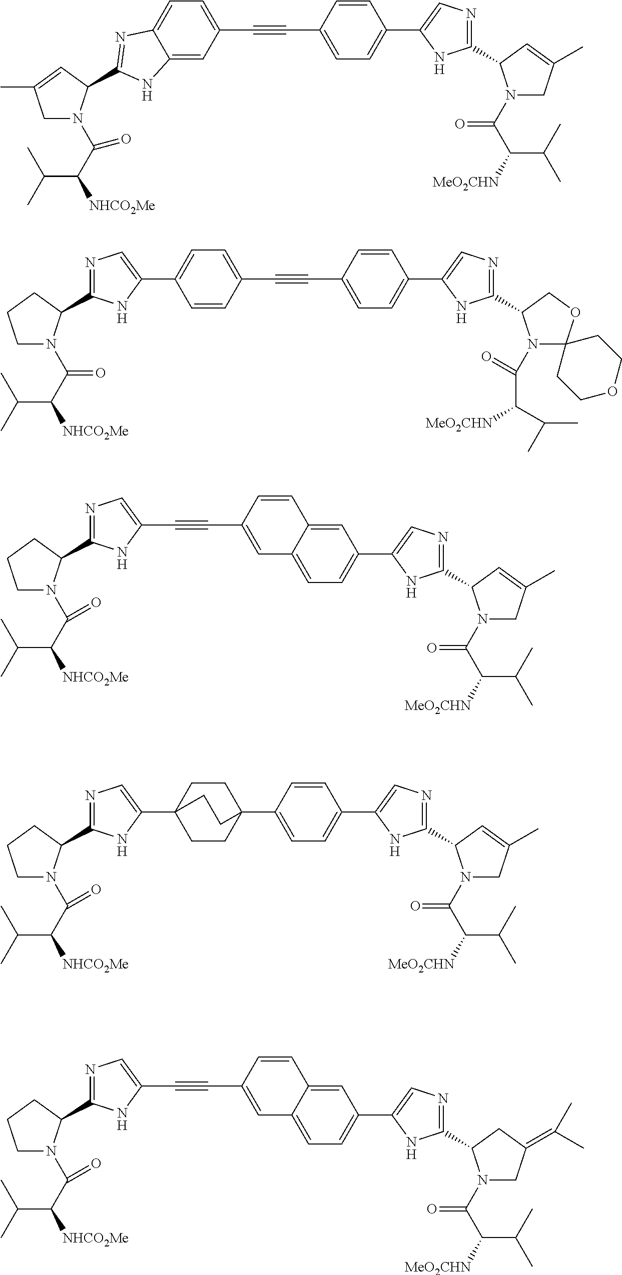 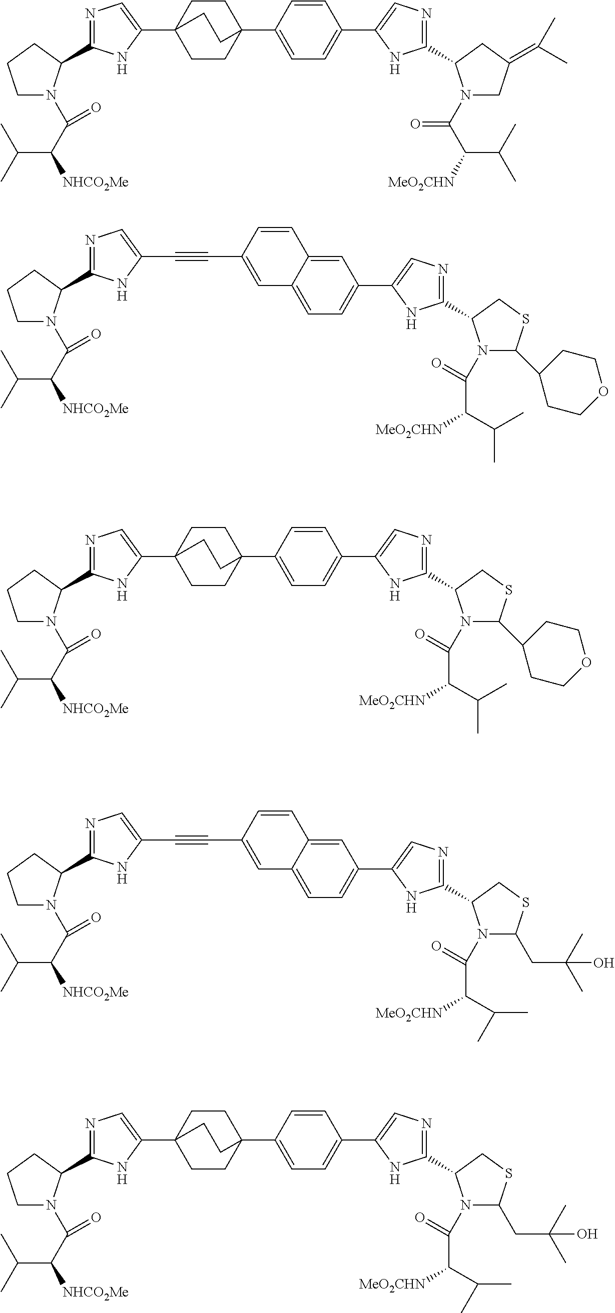 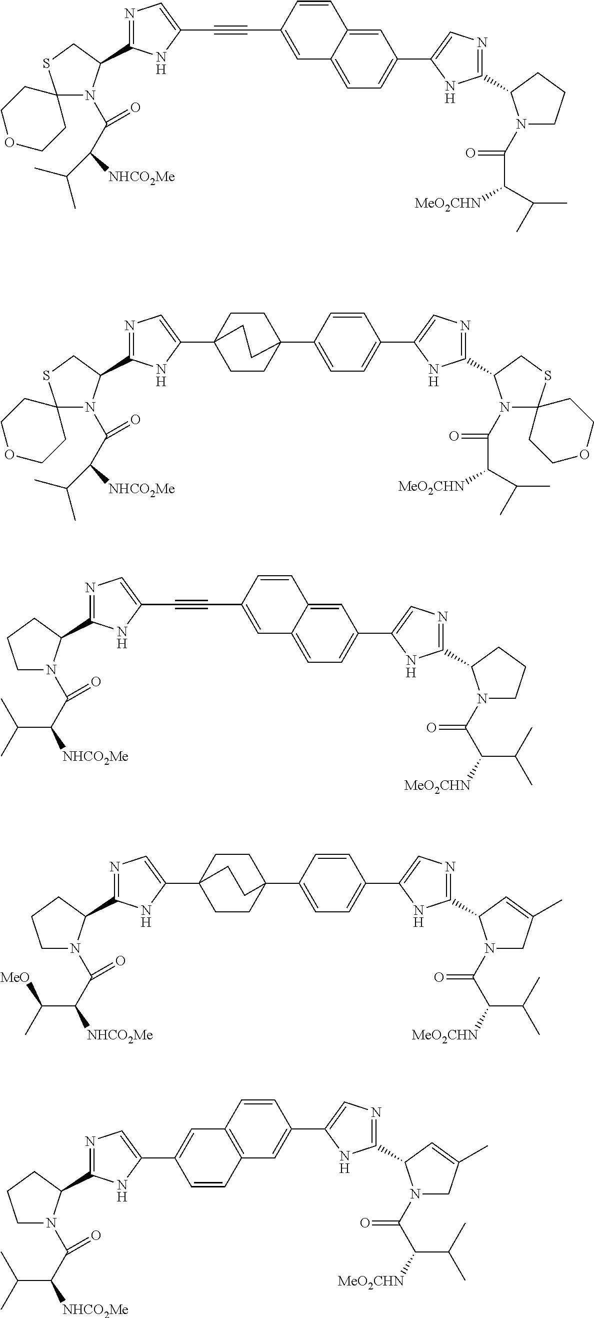 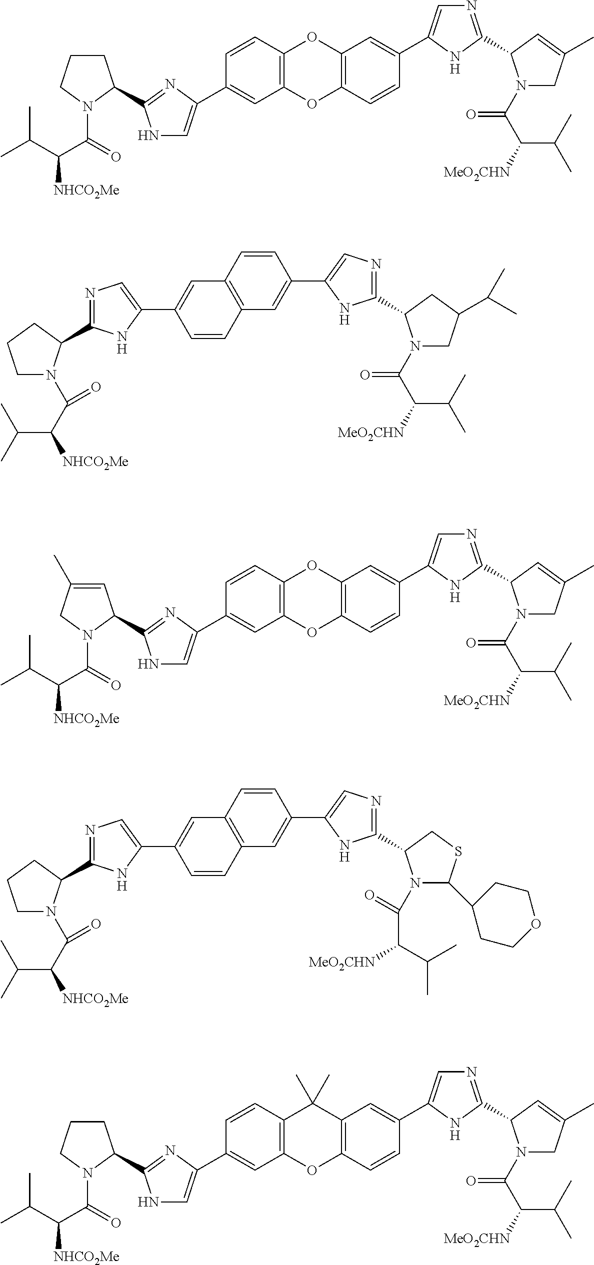 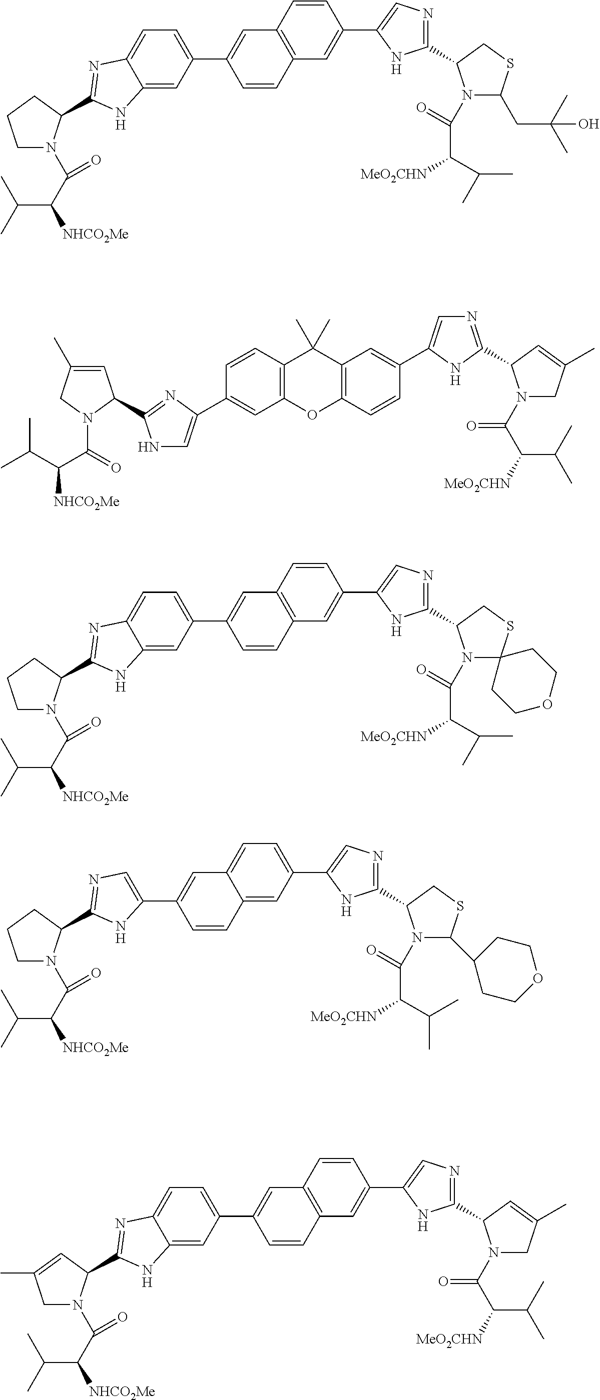 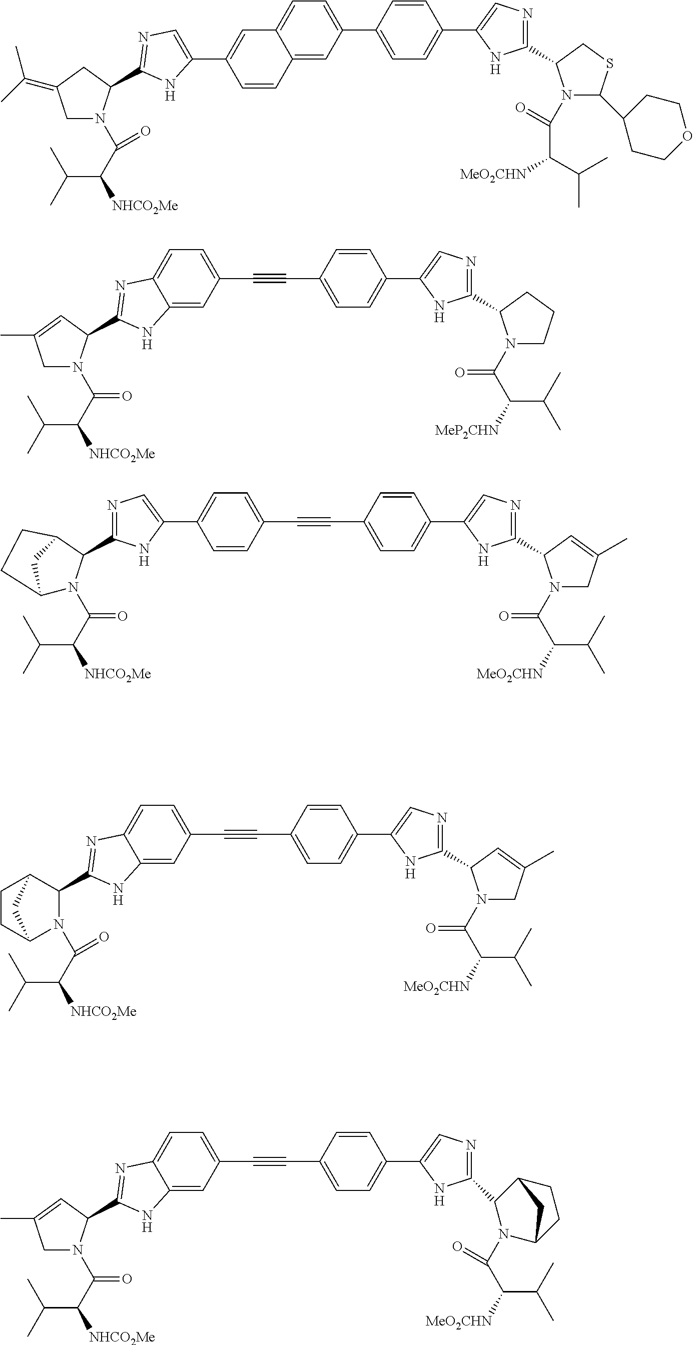 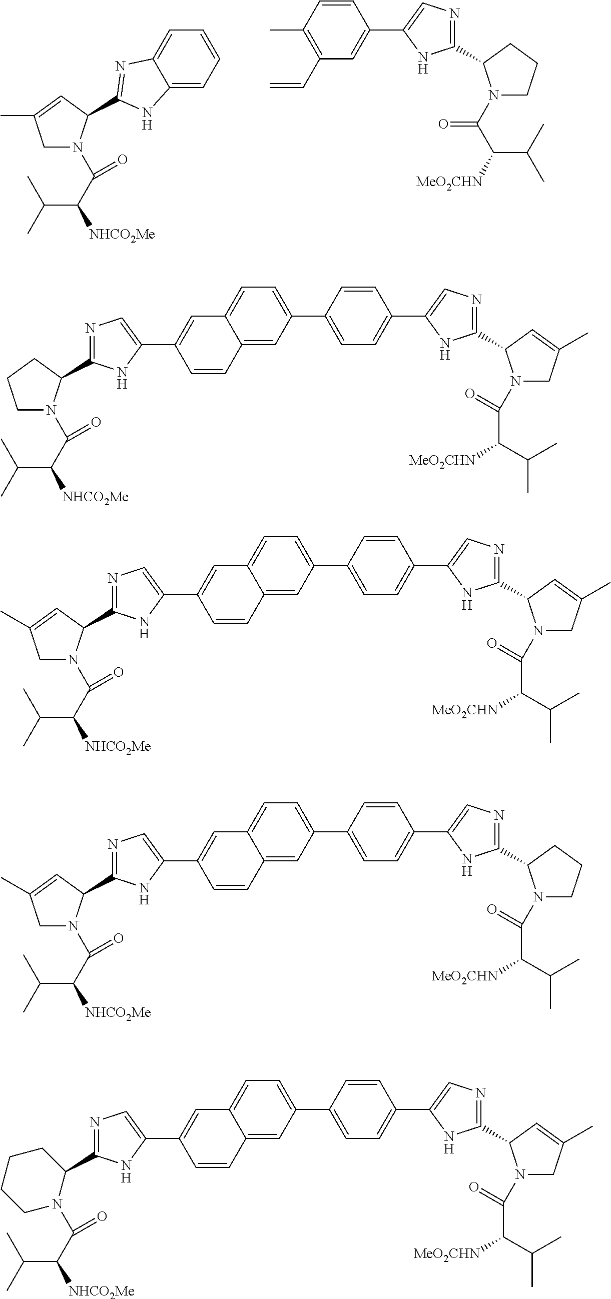 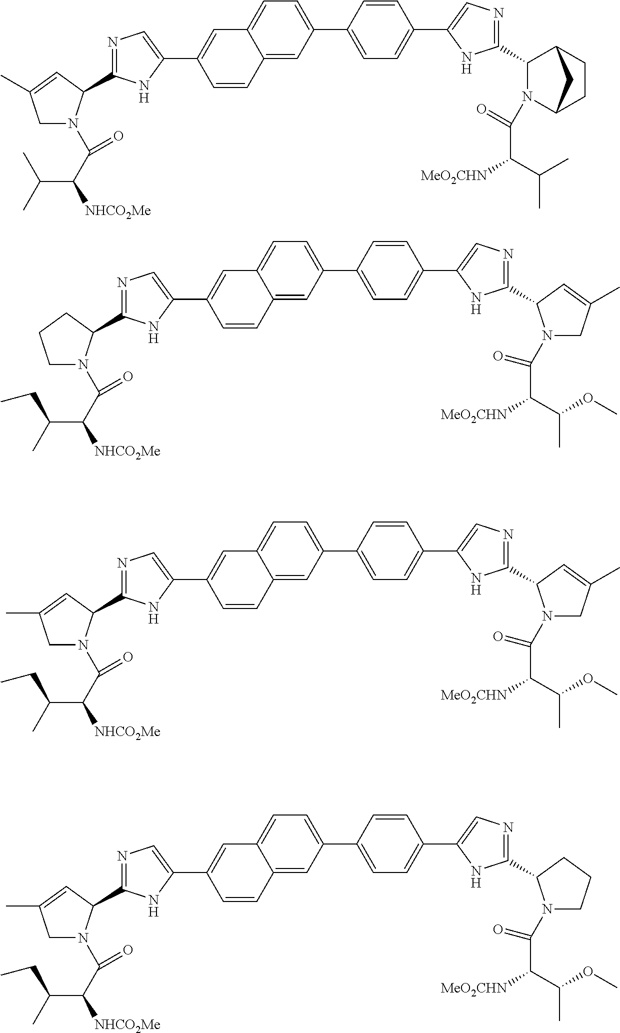 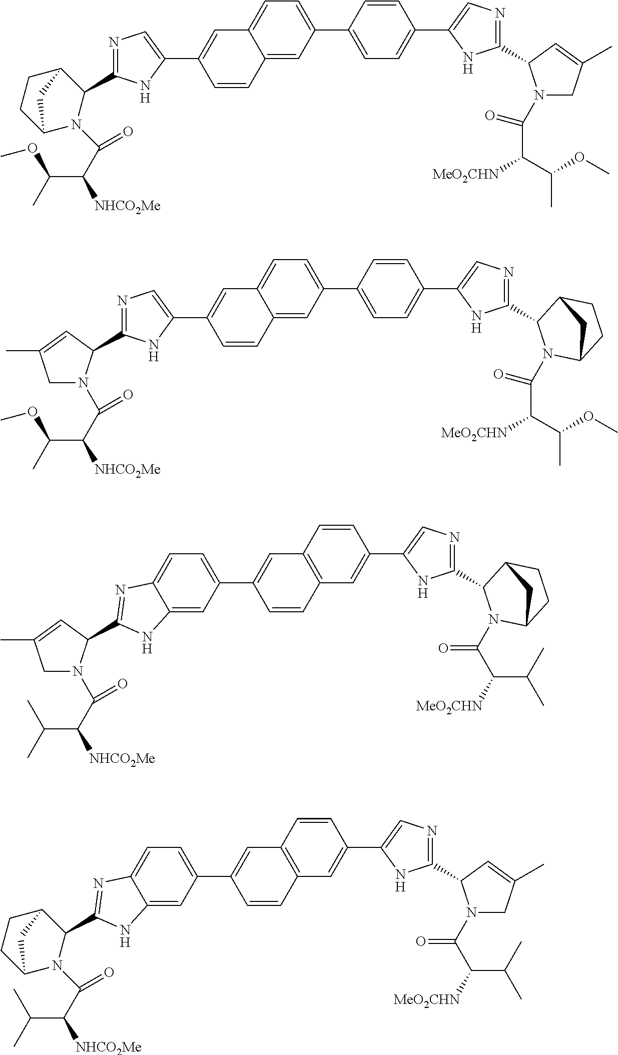 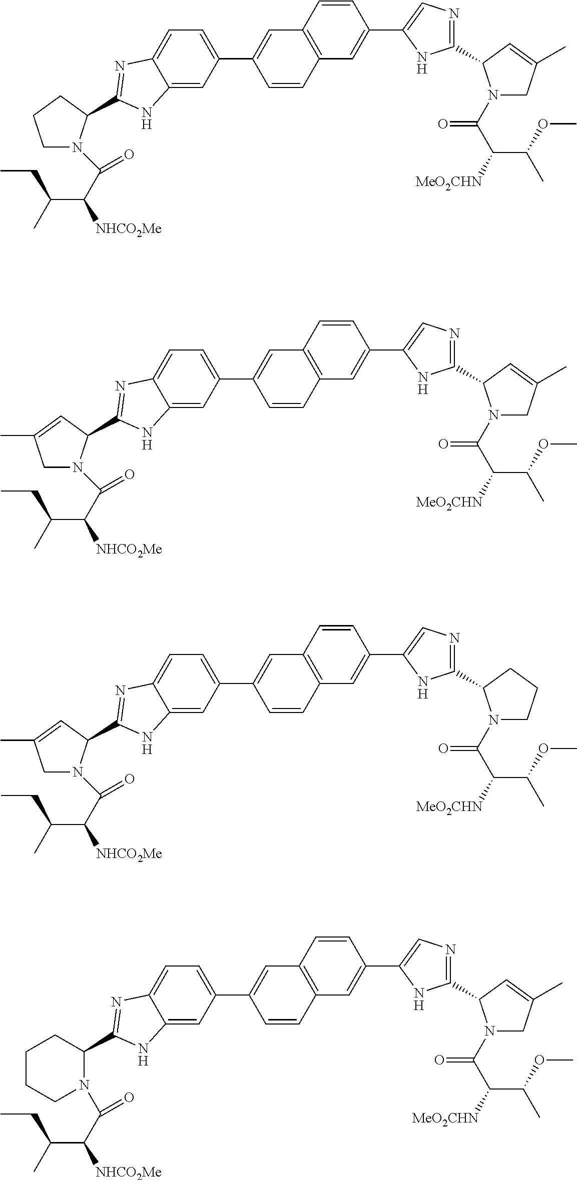 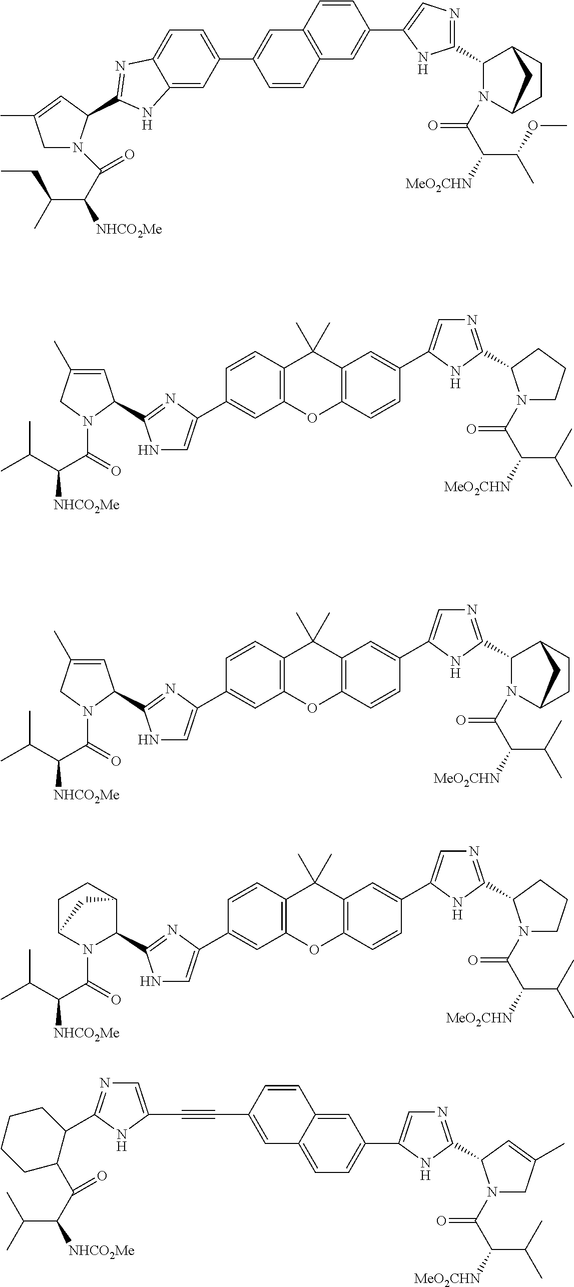 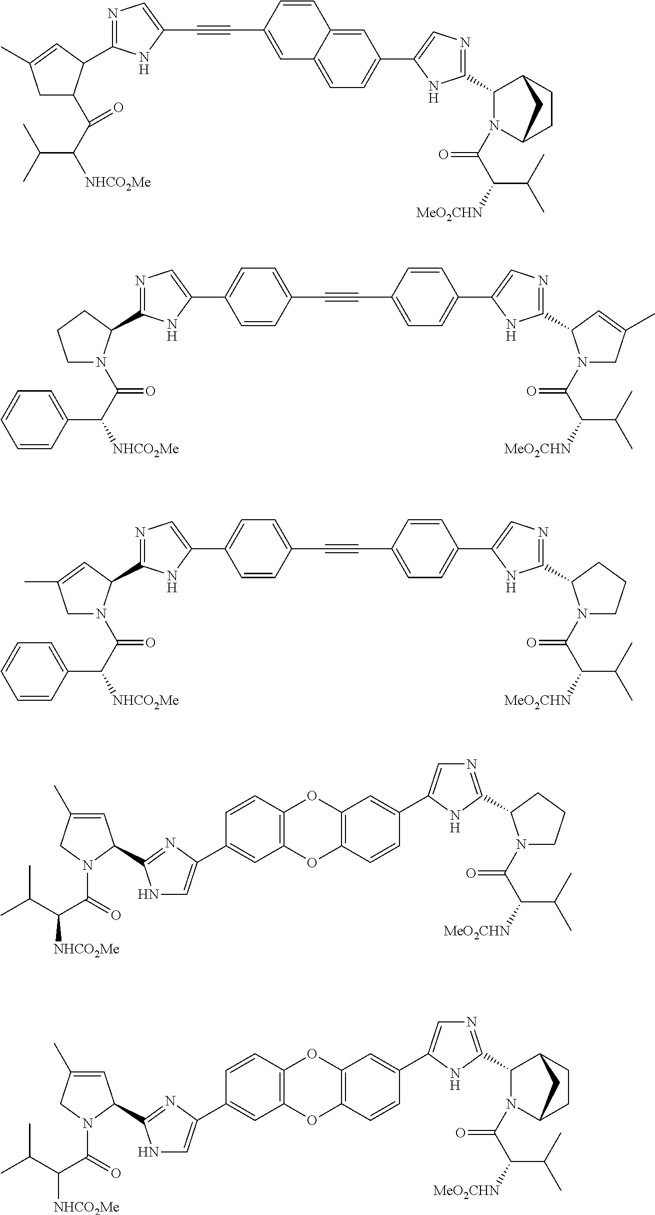 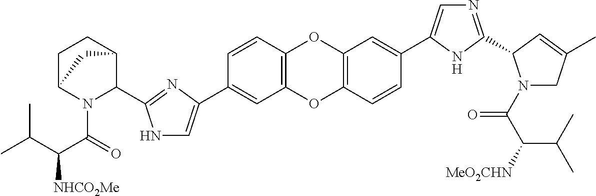
or a pharmaceutically acceptable salt thereof.

32. A pharmaceutical composition comprising a compound of claim 1, or a pharmaceutically acceptable salt thereof, and a pharmaceutically acceptable excipient.

33-34. (canceled)

35. A method of treating hepatitis C comprising administering to a subject in need thereof, a therapeutically effective amount of a compound of claim 1, or a pharmaceutically acceptable salt thereof.
